(19) United States
(12) Patent Application Publication
(10) Pub. No.: US 2026/0263643 A1
(43) Pub. Date: Sep. 10, 2026

(54) BI-SPECIFIC ADAPTERS AND THEIR USE WITH UNIVERSAL CAR-T CELLS IN THE TREATMENT OF CAIX-EXPRESSING TUMORS

(71) Applicant: Purdue Research Foundation, West Lafayette, IN (US)

(72) Inventors: Philip Low, West Lafayette, IN (US); Haiyan A. Chu, Seattle, WA (US); Bo Huang, West Lafayette, IN (US); Sudarsan Kasireddy, West Lafayette, IN (US); Yashapal Singh, Austin, TX (US); Suilan Zheng, West Lafayette, IN (US); Madduri Srinivasarao, West Lafayette, IN (US)

(21) Appl. No.: 19/164,090

(22) PCT Filed: Mar. 11, 2024
(86) PCT No.: PCT/US2024/019323

§ 371 Date: Sep. 10, 2025

Related U.S. Application Data
(60) Provisional application No. 63/451,450, filed on Mar. 10, 2023.
(60) Provisional application No. 63/600,606, filed on Nov. 17, 2023.

(51) Int. Cl. A61K 49/00; A61K 40/11; A61K 40/31; A61P 35/00

(52) U.S. Cl. CPC A61K 49/0052; A61K 40/11; A61K 40/31; A61K 49/0043; A61K 49/0054; A61P 35/00

(57) ABSTRACT

Chimeric antigen receptor (CAR) T-cells, bispecific adapters that link the CAR-T cells to carbonic anhydrase IX (CAIX)-expressing tumor cells, and methods of treating CAIX-expressing cancer using same.

### PRIORITY

This patent application is related to and claims the priority benefit of U.S. Provisional Patent Application No. 63/451,450 filed Mar. 10, 2023, and U.S. Provisional Patent Application No. 63/600,606 filed Nov. 17, 2023. The contents of each of the foregoing applications are hereby incorporated by reference in their entireties into this disclosure.

### TECHNICAL FIELD

The present disclosure relates to chimeric antigen receptor (CAR)-T cells, tumors expressing carbonic anhydrase (CAIX), bispecific adapters that link the CAR-T cells to CAIX-expressing tumor cells, and methods of treating cancer using same.

### BACKGROUND

Carbonic anhydrase IX (CAIX) is upregulated in most solid tumors and is a marker of poor prognosis in at least ovarian, breast, lung, and bladder carcinomas. CAIX is also over-expressed in over 95% of clear cell renal cell carcinomas.

Traditionally, a T-cell expressing a single chimeric antigen receptor (CAR) has been used to target a cell-surface receptor on a tumor cell. When the CAR on the T-cell binds the cell-surface receptor on the tumor cell, the T-cell can kill the tumor cell to which it is bound. While effective, this approach can be quite costly, given that CAR-T cells must be produced for different cancers that express different cell-surface receptors.

Despite the clear need for the prevention and treatment of cancer, it remains a significant cause of death and suffering worldwide because no effective therapeutic options presently exist that can cure the condition. Further, where drugs or other therapies are available, such treatments typically employ highly potent drugs that risk systemic toxicity in the underlying subject as they are poorly selective for the cancer cells of interest.

What is needed are bispecific adapters that can facilitate the cost-efficient use of CAR-T cells that can bind to different cancers that express different cell-surface receptors. This and other objects and advantages, as well as inventive features, will be apparent from the detailed description provided herein.

### SUMMARY

Provided is a bispecific adapter for use with anti-fluorescein chimeric antigen receptor (CAR)-T cells in the treatment of carbonic anhydrase IX (CAIX)-expressing cancer. The bispecific adapter can comprise the following structure.

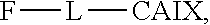

or a pharmaceutically acceptable salt or hydrate thereof, wherein F comprises a fluorescein, L comprises a linker, and CAIX comprises a CAIX ligand (e.g., a radical of a CAIX ligand). The fluorescein can comprise fluorescein, fluorescein isothiocyanate (FITC), or N-hydroxysuccinimide (NHS)-fluorescein. The CAIX ligand can be or comprise a radical of 3-((3-(cyclooctylamino)-2,5,6-trifluoro-4-sulfamoylphenyl)thio)propanoic acid (ortho-CAL). The linker can comprise or consist essentially of or consist of polyethylene glycol (PEG).

The linker can comprise (or consist essentially of or consist of) PEG₃ to PEG₉. The linker can comprise (or consist essentially of or consist of) PEG₆. The linker can comprise (CH₂)4.

In certain embodiments, the bispecific adapter or a pharmaceutically acceptable salt or hydrate thereof comprises a fluorescein, FITC, or NHS-fluorescein conjugated to a radical of a CAIX ligand via a linker, wherein the linker comprises, consists essentially of, or consists of PEG.

The CAIX ligand can be or comprise 3-((3-(cyclooctylamino)-2,5,6-trifluoro-4-sulfamoylphenyl)thio)propanoic acid (ortho-CAL), 3-((2-(cyclooctylamino)-3,5,6-trifluoro-4-sulfamoylphenyl) sulfonyl) propanoic acid (meta-CAL), acetazolamide (Aza), or a derivative or analog of any of the foregoing.

The linker can comprise, consist essentially of, or consist of PEG₁ to PEG₉. The CAIX ligand can be or comprise ortho-CAL or a derivative or analog thereof and the linker can comprise, consist essentially of, or consist of PEG₁ to PEG₉. In certain embodiments, the linker comprises, consists essentially of, or consists of PEG₃ to PEG₉.

In certain embodiments, the CAIX ligand is or comprises meta-CAL or a derivative or analog thereof and the linker comprises, consists essentially of, or consists of PEG₃ to PEG₉.

The CAIX ligand can be or comprise Aza or a derivative or analog thereof. In certain embodiments, the linker comprises, consists essentially of, or consists of PEG₆. In certain embodiments, the linker comprises, consists essentially of, or consists of PEG₉. In certain embodiments, the linker comprises, consists essentially of, or consists of PEG₆.

The linker can comprise, consist essentially of, or consist of an alkyl. The linker can comprise, consist essentially of, or consist of (CH₂)₄.

In certain embodiments, the bispecific adapter comprises a structure of one of the following formulae:

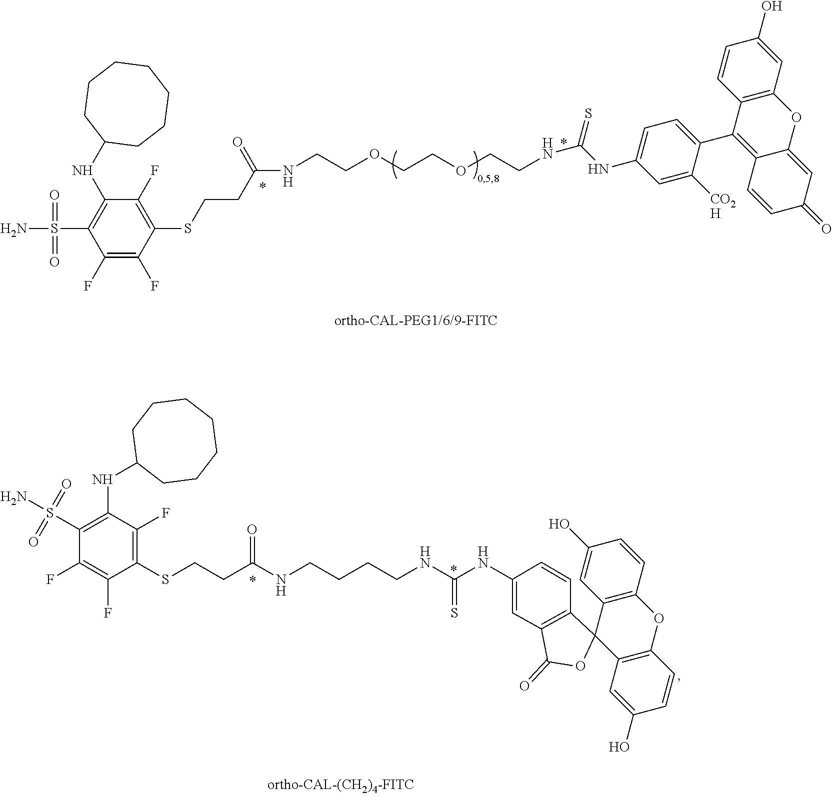

or is a pharmaceutically acceptable salt or hydrate of either of the foregoing.

The bispecific adapter can comprise a structure of the following formulae:

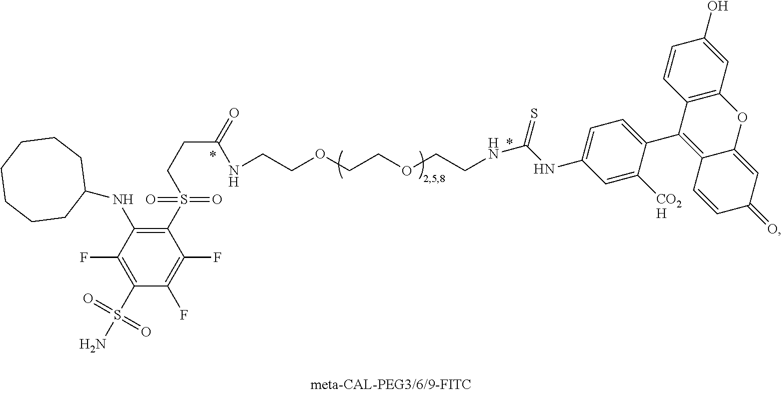

or a pharmaceutically acceptable salt or hydrate thereof.

The bispecific adapter can comprise a structure of the following formulae:

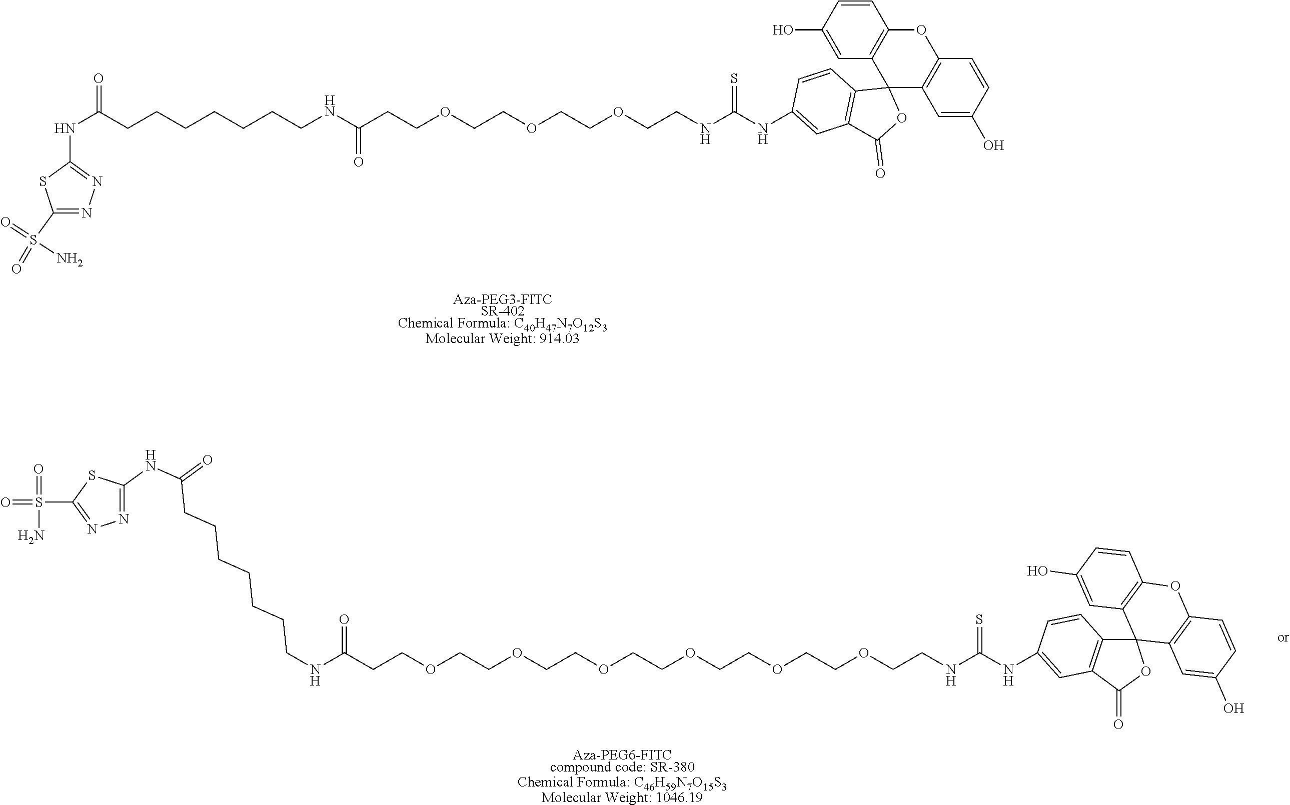

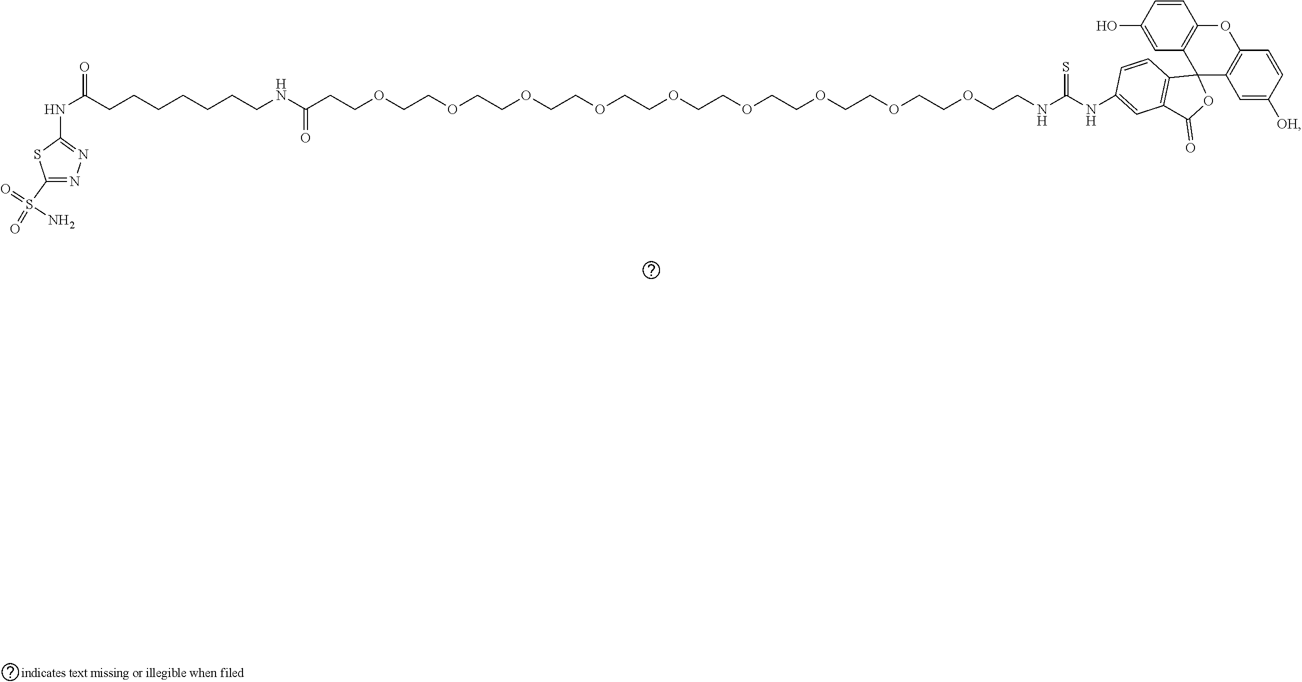 [SMILES: NS(=O)(=O)c1nnc(NC(=O)CCCCCCCNC(=O)CCOCCOCCOCCOCCOCCOCCOCCOCCOCCNC(=S)Nc2ccc3c(c2)C(=O)OC32c3cc(O)ccc3Oc3ccc(O)cc32)s1]

or be a pharmaceutically acceptable salt or hydrate of any of the foregoing.

The bispecific adapters can be used with an anti-fluorescein chimeric antigen receptor (CAR)-T cell in the treatment of cancer. The bispecific adapters can be used with an anti−

In certain embodiments, the bispecific adapter is for use with an anti-fluorescein (e.g., fluorescein, FITC, or NHS-fluorescein) CAR-T cell in the treatment of CAIX-expressing cancer. The bispecific adapter can comprise fluorescein-linker-CAIX ligand (e.g., fluorescein=fluorescein, FITC, or NHS-fluorescein) or a pharmaceutically acceptable salt or hydrate thereof, wherein the CAIX ligand is or comprises 3-((2-(cyclooctylamino)-3,5,6-trifluoro-4-sulfamoylphenyl)sulfonyl)propanoic acid (meta-CAL) and the linker comprises (or consists essentially of or consists of) PEG. The linker can comprise (or consist essentially of or consist of) PEG₃ to PEG₉. The linker can comprise (or consist essentially of or consist of) PEG₆. The linker can comprise (or consist essentially of or consist of) PEG₉. The bispecific adapter can have the structure:

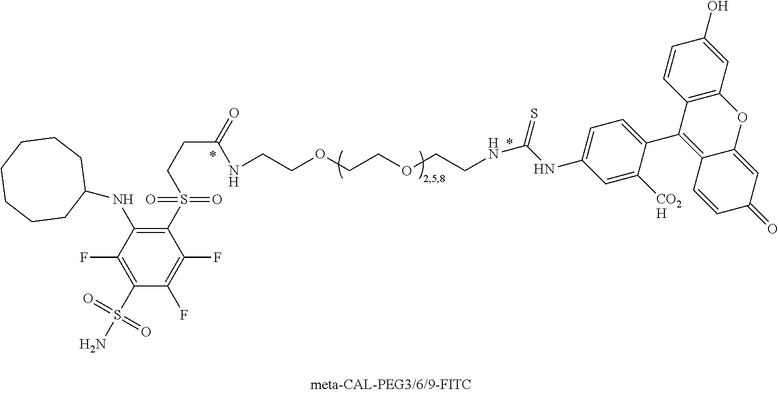

or
be a pharmaceutically acceptable salt or hydrate thereof.

Provided is yet another bispecific adapter for use with an anti-fluorescein (e.g., fluorescein, FITC, or NHS-fluorescein) CAR-T cell in the treatment of CAIX-expressing cancer. The adapter can comprise fluorescein-linker-CAIX ligand (e.g., fluorescein=fluorescein, FITC, or NHS-fluorescein) or a pharmaceutically acceptable salt or hydrate thereof, wherein the CAIX ligand is acetazolamide (Aza) and the linker comprises (or consists essentially of or consists of) PEG. The linker can comprise (or consist essentially of or consist of) PEG₁ to PEG₉. The linker can comprise (or consist

Also provided is a pharmaceutical composition for the treatment of CAIX-expressing cancer comprising any bispecific adapter described herein and a pharmaceutically acceptable carrier or excipient.

Further provided is a method of treating a cancer (e.g., a CAIX-expressing cancer) in a subject. The method can comprise administering to the subject cancer-treatment effective amounts of (i) anti-fluorescein (e.g., fluorescein, FITC, or NHS-fluorescein) CAR-T cells or a pharmaceutical composition comprising anti-fluorescein CAR-T cells and a pharmaceutically acceptable carrier or excipient; and (ii) a herein-described bispecific adapter or a pharmaceutical composition comprising a herein-described bispecific adapter and a pharmaceutically acceptable carrier or excipient, whereupon the subject is treated for cancer. The CAR can have a recognition region and the recognition region can be a single chain fragment variable (scFv) region of an anti-fluorescein antibody. The CAR can have a co-stimulation domain. The co-stimulation domain of the CAR can be CD28, CD137 (4-1BB), CD134 (OX40), or CD278 (ICOS). The CAR can have an activation signaling domain such as, for example, a T cell CD3 chain or an Fc receptor γ.

The fluorescein of the bispecific adapter of the method can bind the anti-fluorescein CAR-T cell with affinity upon exposure thereto, and the CAIX ligand of the bispecific adapter can link the bound anti-fluorescein CAR-T cell to a CAIX-expressing cancer cell upon the bispecific adapter binding a receptor on such CAIX-expressing cancer cell with affinity.

In the method, steps (i) and (ii) can be administered simultaneously or sequentially, in either order, by the same or different routes. In the method, steps (i) and (ii) can each be administered intravenously.

In certain embodiments, the method can further comprise imaging the cancer in the subject. Imaging the cancer can comprise imaging by optical imaging, positron emission tomography (PET), or single photon emission computed tomography (SPECT).

The cancer can be ovarian cancer, endometrial cancer, breast cancer, lung cancer, bladder cancer, or clear cell renal cell carcinoma such as, optionally, stage 3-4 clear cell renal cell carcinoma. The cancer can be a CAIX-expressing cancer.

Methods for enhancing CAR-T cell activation are also provided. In certain embodiments, a method for enhancing CAR-T cell activation comprises: providing a herein-described bispecific adapter or a pharmaceutical composition described herein; and exposing anti-fluorescein CAR-T cells or a pharmaceutical composition comprising anti-fluorescein CAR-T cells and a pharmaceutically acceptable carrier or excipient to the bispecific adaptor or pharmaceutical composition; wherein the CAR-T cell experiences enhanced activation against cancer cells following exposure as compared to a CAR-T cell not exposed to the bispecific adapter.

The CAR-T cells can be in systemic circulation in a subject when exposed to the bispecific adapter. The CAR-T cells can be exposed to the bispecific adapter in vitro (e.g., prior to administration to a subject). The CAR-T cells can be CAIX-expressing cancer cells.

Still further provided is a kit. The kit can comprise (i) at least one dosage unit of a herein-described bispecific adapter or a pharmaceutical composition comprising the same and a pharmaceutically acceptable carrier or excipient, and (ii) at least one dosage unit of anti-fluorescein CAR-T cells or a pharmaceutical composition comprising the same and a pharmaceutically acceptable carrier or excipient, wherein (i) and (ii) are optionally in separate containers. The CAIX ligand of the bispecific adapter can be or comprise ortho-CAL or a derivative or analog thereof. The CAIX ligand of the bispecific adapter can be or comprise meta-CAL or a derivative or analog thereof. The CAIX of the bispecific adapter can be or comprise Aza or a derivative or analog thereof.

### DESCRIPTION OF THE DRAWINGS

The above and other objects, features, and advantages of the present invention will become more apparent when taken in conjunction with the following description and drawings, wherein:

FIG. 1 shows the chemical structures of carbonic anhydrase IX (CAIX)-targeting bispecific adapters.

FIG. 2A is a graph of concentration (nM) of the indicated bispecific adapters vs. mean fluorescence intensity (MFI).

FIG. 2B is a graph of concentration (nM) of the indicated bispecific adapters vs. MFI.

FIG. 3A is a graph of bispecific adapter vs. MFI of fluorescein isothiocyanate (FITC), which shows the total binding of CAIX bispecific adapters to HT29 cells.

FIG. 3B is a graph of bispecific adapter vs. MFI of APC-anti-FITC, which shows the surface exposure of FITC moieties in CAIX bispecific adapters after binding to HT29 cells.

FIG. 3C is a graph of bispecific adapter vs. MFI of FITC, which shows the total binding of CAIX-bispecific adapters to MDA-CAIX cells.

FIG. 3D is a graph of bispecific adapter vs. MFI of APC-anti-FITC, which shows the surface exposure of FITC moieties in CAIX-bispecific adapters after binding to MDA-CAIX cells.

FIG. 4A is a graph of concentration (nM) vs. lysis (%), which shows CAIX-targeting bispecific adapter-mediated anti-FITC CAR-T cell cytotoxicity to MDA-CAIX cells.

FIG. 4B is a graph of concentration (nM) vs. IFNγ (pg/ml), which shows bispecific adapter-mediated IFNγ release from anti-FITC CAR-T cells.

FIG. 5A is a graph of concentration (nM) vs. lysis (%), which shows meta-CAL-FITC bispecific adapters mediated anti-FITC CAR-T cell cytotoxicity to MDA-CAIX cells.

FIG. 5B is a graph of concentration (nM) vs. IFNγ (pg/ml), which shows meta-CAL-PEG₆-FITC and meta-CAL-PEG₉-FITC mediated higher levels of IFNγ release from anti-FITC CAR-T cells than meta-CAL-PEG₃-FITC at low concentrations (between 0.001 nM and 1 nM).

FIG. 6A is a graph of concentration (nM) vs. lysis (%), which shows ortho-CAL-FITC bispecific adapter with different linkers mediated anti-FITC CAR-T cell cytotoxicity to MDA-CAIX cells.

FIG. 6B is a graph of concentration (nM) vs. IFNγ (pg/ml), which shows ortho-CAL-FITC bispecific adapters mediated IFNγ release from anti-FITC CAR-T cells.

FIG. 7A shows the timeline and dosing schedule of an in vivo study to test different CAIX bispecific adapters.

FIG. 7B is a graph of days post-CAR-T injection vs. tumor volume (mm³), which shows tumor growth curves of different treatment groups.

FIG. 7C is a graph of days post-CAR-T injection vs. body weight change (%), which shows body weight changes of the mice in different treatment groups over the course of treatment.

FIG. 8A is a schematic diagram of an experimental design.

FIG. 8B is a graph of bispecific adapter vs. MFI of FITC, which shows the total FITC signal in HT29 tumor cells from mice injected with different bispecific adapters.

FIG. 8C is a graph of bispecific adapter vs. MFI of APC-anti-FITC, which shows the surface exposure of the FITC moiety I nHT29 tumors from mice injected with CAIX bispecific adapters.

FIG. 9A is a graph of ligand vs. T cell/μL blood, which shows the T cell counts in blood.

FIG. 9B is a graph of ligand vs. CD3+ T cells/live cells (%), which shows tumor-infiltrated T cells.

FIG. 10A shows the timeline and dosing schedule of an in vivo study to test different CAIX bispecific adapters.

FIG. 10B is a graph of days post-CAR-T injection vs. tumor volume (mm³), which shows tumor growth curves of different treatment groups.

FIG. 10C is a graph of days post-CAR-T injection vs. body weight change (%), which shows body weight changes of the mice in different groups during treatments.

FIG. 11A is a schematic diagram of experimental design.

FIG. 11B is a graph of bispecific adapter vs. MFI of FITC, which shows the total FITC signal in the MDA-CAIX tumor cells from mice injected with different bispecific adapters.

FIG. 11C is a graph of bispecific adapter vs. MFI of FITC after staining with 100 nM meta-CAL-PEG₆-FITC in vitro, which shows the total FITC signal in the tumor cells. In vitro staining of the cells with 100 nM meta-CAL-PEG₆-FITC would saturate all the surface CAIX proteins and reflect the CAIX protein level on the tumor cells. The results indicate that orthoCAL-PEG₆-FITC mediated complete eradication of CAIX⁺ tumor cells by CAR T cells.

FIG. 11D is a graph of bispecific adapter vs. MFI of APC-anti-FITC, which shows the surface exposure of the FITC moiety in MDA-CAIX tumors from mice treated with CAIX bispecific adapters.

FIG. 11E is a graph of bispecific adapter vs. MFI of APC-anti-FITC after staining with 100 nM meta-CAL-PEG₆-FITC in vitro, which shows the total FITC signal in the tumor cells. In vitro staining of the cells with 100 nM meta-CAL-PEG₆-FITC would saturate all the surface CAIX proteins and reflect the CAIX protein level on the tumor cells.

FIG. 12 is a graph of concentration (nM) vs. normalized MFI, which shows that PEG spacers do not have significant effects on the binding affinity of acetazolamide (Aza)-FITC bispecific adapters.

FIG. 13A is a graph of bispecific adapter vs. MFI of FL, which shows the total binding of Aza-FITC bispecific adapters to MDA-CAIX cells. Linker length did not significantly affect the total binding.

FIG. 13B is a graph of bispecific adapter vs. MFI of APC-anti-FITC, which shows the surface exposure of FITC moieties in Aza-FITC bispecific adapters with different PEG linkers after binding to MDA-CAIX cells. Increased PEG spacer length enhanced surface exposure of FITC after binding to MDA-CAIX cells.

FIG. 14 is a graph of concentration (nM) vs. IFNγ (pg/ml), which shows the effect of linker length on Aza-FITC bispecific adapter mediated anti-FITC CAR-T cell IFNγ release when co-cultured with HT 29 cells. Aza-PEG3-FITC and Aza-PEG₉-FITC mediated higher level of IFNγ released from anti-FITC CAR-T cells than Aza-PEG₀-FITC. Aza-PEG₆-FITC mediated the highest level of IFNγ released from anti-FITC CAR-T cells.

FIG. 15 is a graph of concentration of CA9 ligand-PEG(n)-FITC (nM) vs. IFNγ (pg/ml), which shows CAIX-targeting bispecific adapters with optimal linkers mediated anti-FITC CAR-T cell IFNγ release. MetaCAL-PEG9-FITC mediated the highest level of IFNγ released from anti-FITC CAR-T cells at low concentrations (<0.1 nM). Aza-PEG₆-FITC mediated similar levels of IFNγ release from anti-FITC CAR T-cells as metaCAL-PEG₀-FITC at concentrations higher than 0.1 nM. OrthoCAL-PEG₀-FITC mediated lower levels of IFNγ released from anti-FITC CAR-T cells.

FIG. 16A shows the timeline and dosing schedule of an in vivo study to test Aza-PEG₆-FITC and orthoCAL-PEG₆-FITC.

FIG. 16B is a graph of days post-CAR-T cell injection vs. tumor volume (mm³), which shows tumor growth curves of different treatment groups. Aza-PEG₆-FITC and orthoCAL-PEG₆-FITC both slightly inhibited the growth of KB tumors. The efficacy of Aza-PEG₆-FITC is slightly better than that of orthoCAL-PEG₆-FITC.

FIG. 16C is a graph of days post-CAR-T cell injection vs. body weight change (%), which shows body weight changes of mice in different treatment groups. Both Aza-PEG₆-FITC and orthoCAL-PEG₆-FITC didn't induce significant body weight loss.

### DETAILED DESCRIPTION

The present disclosure is predicated, at least in part, on the design of bispecific adapters with various antigen binding affinities and spacer lengths to optimize universal chimeric antigen receptor (CAR)-T cell efficacy in the treatment of carbonic anhydrase IX (CAIX)-expressing tumors. Two new, high-affinity CAIX ligands, namely 3-((2-(cyclooctylamino)-3,5,6-trifluoro-4-sulfamoylphenyl) sulfonyl) propanoic acid (meta-CAL) and 3-((3-(cyclooctylamino)-2,5,6-trifluoro-4-sulfamoylphenyl) thio) propanoic acid (ortho-CAL), were tested with three or four spacer linkers, respectively, in bispecific adapters. The studies supported that PEG₆ and PEG₉ (PEG=polyethylene glycol) spacers can be optimal in targeting CAIX-positive tumors in vivo and in vitro. When used in the bispecific adapters, the high-affinity CAIX ligands were more potent than adapters with low affinity, such as 5-acetamido-1,3,4-thiadiazole-2-sulfonamide (Aza).

In view of the above, provided is a bispecific adapter or a pharmaceutically acceptable salt or hydrate thereof for use with anti-fluorescein (e.g., fluorescein, FITC, or N-hydroxysuccinimide (NHS)-fluorescein) CAR-T cells in the treatment of CAIX-expressing cancer.

In certain embodiments, the bispecific adapter comprises the following structure:

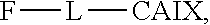

or is a pharmaceutically acceptable salt or hydrate thereof, wherein:
F comprises a CAR-T cell targeting moiety such as, for example, fluorescein, FITC, or NHS-fluorescein,
L comprises a linker, and
CAIX comprises a radical of a CAIX ligand.

In certain embodiments, the CAIX ligand is or comprises 3-((3-(cyclooctylamino)-2,5,6-trifluoro-4-sulfamoylphenyl)thio)propanoic acid (ortho-CAL) and the linker comprises (or consists essentially of or consists of) PEG.

The use of bispecific adapters can enable the use of a single CAR-T cell. i.e., a “universal” CAR-T cell, that displays, for example, a molecule on its surface that binds fluorescein. When the universal CAR-T cell, such as one displaying a molecule on its surface that binds fluorescein, is used in conjunction with a bispecific adapter, such as one comprising FITC connected (e.g., by a linker and/or spacer) to a molecule, which binds a cell-surface receptor of tumor cells, the T-cell can kill the tumor cells to which it is bound.

This approach can reduce the cost of producing CAR-T cells that can bind different cancers that express different cell-surface receptors. Instead, the universal CAR-T cell can bind to different types of cancers by changing the part of the bispecific adapter that binds a cell-surface receptor on a tumor cell. Accordingly, the bispecific adapters can improve the immune response effected by CAR-T cells bound to tumor cells.

### CAR T-Cell Targeting Moiety

The CAR T-cell targeting moiety of the bispecific adapter can be fluorescein, FITC, NHS-fluorescein, or any other moiety that a CAR can be engineered to recognize and bind with specificity.

“Binds with specificity.” “binds with high affinity,” or “specifically” or “selectively” binds, when referring to a ligand/receptor, a recognition region/targeting moiety, a nucleic acid/complementary nucleic acid, an antibody/antigen, or other binding pair indicates a binding reaction that is determinative of the presence of the protein in a heterogeneous population of proteins and other biologics. Thus, under designated conditions, a specified ligand or recognition region binds to a particular receptor (e.g., one present on a cancer cell or CAR T-cell) or targeting moiety, respectively, and does not bind in a significant amount to other proteins present in the sample (e.g., those associated with normal, healthy cells). Specific binding or binding with high affinity can also mean, for example, that the binding compound, ligand, antibody, or binding composition derived from the antigen-binding site of an antibody binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound. In a typical embodiment, a molecule that specifically binds a target will have an affinity that is at least about 10⁶ liters/mol (K_{D}=10⁻⁶ M), and preferably at least about 10 liters/mol, as determined, for example, by Scatchard analysis.

### Targeting Ligands

As noted above, the bispecific adapters can comprise a radical of a CAIX ligand. When administered, the targeting ligand targets the bispecific adapter conjugate to a cancer or tumor of interest that expresses the associated receptor. In some embodiments, the targeting moieties (in their free form, a radical thereof) do not bind with uptake receptors on non-targeted cells.

CAIX is a small molecule ligand that binds with specificity to a receptor that is overexpressed on certain cancer cell types (i.e., the receptor for each of these ligands is overexpressed on cancers as compared to expression of such receptor on normal tissues or, potentially, in diseased tissue not experiencing the targeted cancer type). Receptors for the CAIX ligand are found, for example, on renal, ovarian, vulvar, and breast cancer, and cancers of the colon and pancreas. Its expression can also be associated with renal cell carcinoma, lung cancer, and others. Accordingly, upregulated CAIX expression can be a useful target for therapy.

The bispecific adapters can comprise a CAIX ligand (or a radical thereof) attached to a linker, wherein the linker is further attached to a CAR-targeting moiety. In certain embodiments, the CAIX ligand is a high affinity CAIX ligand. Unless otherwise specified, “high affinity” or “higher affinity” with respect to a ligand's affinity for a target means a ligand that has a Schrodinger molecular docking score of at least about −8.0 kcal/mol. In certain embodiments, the high affinity CAIX ligand has an improved affinity for the CAIX receptor as compared to other ligands.

The targeting moiety can be, for example, a radical of CAIX ligand with a molecular weight less than about 10,000, less than 7,500, less than 5,000, less than 2,500, less than 1,000, less than 750, less than 500; from about 500 to about 10,000 g/mol, about 1,000 to about 7,500 g/mol, about 750 g/mol to about 1,500 g/mol, about 1,000, to about 5,000 g/mol or about 500 to about 2,500 g/mol.

The targeting ligand can bind to an activated tumor or other cancer cell that is overexpressing the CAIX receptor. In certain embodiments, the targeting ligand can have a binding affinity to a CAIX receptor in the range between about 1 nM to about 25 nM, such as 1 nM to about 25 nM or about 1 nM to 25 nM. In certain embodiments, the targeting ligand can have a binding affinity to a CAIX receptor in the range between about 0.002 nM to about 25 nM, such as 0.002 nM to about 1 nM or about 0.002 nM to 1 nM. In certain embodiments, the targeting ligand can have a binding affinity to a CAIX receptor in the range between about 0.01 nM to about 0.9 nM, such as 0.01 nM to about 0.9 nM or about 0.01 nM to 0.9 nM. In certain embodiments, the targeting ligand can have a binding affinity to a CAIX receptor in the range between about 0.02 nM to about 0.8 nM, such as 0.02 nM to about 0.8 nM or about 0.02 nM to 0.8 nM. In certain embodiments, the targeting ligand can have a binding affinity to a CAIX receptor in the range between about 0.03 nM to about 0.7 nM, such as 0.03 nM to about 0.7 nM or about 0.03 nM to 0.7 nM. In certain embodiments, the targeting ligand can have a binding affinity to a CAIX receptor in the range between about 0.04 nM to about 0.6 nM, such as 0.04 nM to about 0.6 nM or about 0.04 nM to 0.6 nM. In certain embodiments, the targeting ligand can have a binding affinity to a CAIX receptor in the range between about 0.05 nM to about 0.5 nM, such as 0.05 nM to about 0.5 nM or about 0.05 nM to 0.5 nM. In certain embodiments, the targeting ligand can have a binding affinity to a CAIX receptor in the range between about 0.06 nM to about 0.4 nM, such as 0.06 nM to about 0.4 nM or about 0.06 nM to 0.4 nM. In certain embodiments, the targeting ligand can have a binding affinity to a CAIX receptor in the range between about 0.07 nM to about 0.3 nM, such as 0.07 nM to about 0.3 nM or about 0.07 nM to 0.3 nM. In certain embodiments, the targeting ligand can have a binding affinity to a CAIX receptor in the range between about 0.08 nM to about 0.2 nM, such as 0.08 nM to about 0.2 nM or about 0.08 nM to 0.2 nM. In certain embodiments, the targeting ligand can have a binding affinity to a CAIX receptor in the range between about 0.09 nM to about 0.1 nM, such as 0.09 nM to about 0.1 nM or about 0.09 nM to 0.1 nM. The ranges set forth in this section are inclusive of the stated endpoints and all 0.001 nM increments encompassed thereby.

The CAIX ligand can be or can comprise 3-((3-(cyclooctylamino)-2,5,6-trifluoro-4-sulfamoylphenyl)thio)propanoic acid (ortho-CAL) or derivatives or analogs thereof. The CAIX ligand can be or can comprise 3-((2-(cyclooctylamino)-3,5,6-trifluoro-4-sulfamoylphenyl)sulfonyl)propanoic acid (meta-CAL) or derivatives or analogs thereof. The CAIX ligand can be or can comprise acetazolamide (Aza) or derivatives or analogs thereof. In certain embodiments, the CAIX ligand is selected from the group consisting of meta-CAL, ortho-CAL, and Aza. When administered, a bispecific adapter conjugate comprising a CAIX ligand or radical thereof can target a CAIX-expressing cancer in a subject.

The bispecific adapter can be specifically designed and synthesized to achieve a particular binding affinity for CAIX.

### Linkers

The linkers of the bispecific adapters hereof are disposed between the targeting ligand (e.g., a radical thereof) and the CAR T-cell targeting moiety (for example, comprising fluorescein, FITC, or NHS-fluorescein). The linker can be any suitable linker.

The term “linker” includes a chain of atoms that is bio-functionally adapted to form a chemical bond and connects the CAR T-cell targeting moiety and the cancer-targeting ligand to form a conjugate. Illustratively, the chain of atoms can include carbon, nitrogen, oxygen, sulfur, silicon (Si), and phosphorus (P), such as C, N, O, S, and P, or C, N, O, and S.

The linker can comprise a wide variety of links, such as in the range from about 2 to about 100 atoms in the contiguous backbone. The linker can comprise a releasable form of PEG, a non-releasable form of PEG, polyproline, a hydrophilic amino acid, a sugar, an unnatural peptidoglycan, polyvinylpyrrolidone, or a triblock copolymer comprising a central hydrophobic block of polypropylene glycol flanked on each side by a hydrophilic block of PEG.

The linker can comprise PEG or a PEG derivative. The linker can be (PEG)₃.

The linker can be non-releasable, i.e., non-labile. However, in some embodiments, it may be desirable for a linker in a bispecific adapter to be releasable, i.e., labile, such as, for example, photocleavable, acid-labile, base-labile, or enzyme-cleavable. The term “releasable” in the context of a linker means a linker that includes at least one bond that can be easily broken (e.g., chemically or enzymatically hydrolyzed) under physiological conditions, such as, for example, by reducing agent-labile, pH-labile, acid-labile, base-labile, oxidatively labile, metabolically labile, biochemically labile, enzyme-labile or p-aminobenzylic-based multivalent releasable bond. It is appreciated that the physiological conditions resulting in bond breaking do not necessarily include a biological or metabolic process and instead can include a standard chemical reaction, such as a hydrolysis reaction for example, at physiological pH or as a result of compartmentalization into a cellular organelle, such as an endosome having a lower pH than cytosolic pH. A cleavable bond can connect two adjacent atoms within the releasable linker and/or connect other linker portions or the targeting moiety and/or CAR T-cell targeting moiety, as described herein, for example, at either or both ends of the releasable linker. In some instances, the releasable linker is broken into two or more fragments. In some instances, the releasable linker is separated from the CAR T-cell targeting moiety.

In some embodiments, the linker is formed such that the CAR T-cell targeting ligand (i.e., the fluorescein) is cleaved from the cancer-targeting moiety (i.e., the CAIX ligand) only after sufficient time has passed for the bispecific adapter to circulate within a subject's systemic circulation following administration (e.g., to allow time to be captured and internalized by the targeted cell and/or receptor). In some embodiments, the time period for the release will vary (e.g., from subject to subject (e.g., based on a variety of factors)). In some embodiments, a releasable linker can be engineered such that it will not cleave/release until at least 24 hours post-administration or even over a period of a week. In some embodiments, the bispecific adapter can safely pass through the subject's system, and any amount not captured by the targeted cells (e.g., those expressing CAIX, for example) can be excreted.

In contrast, the term “non-releasable” in the context of a linker means a linker that includes at least one bond that is not easily or quickly broken under physiological conditions. In some embodiments, a non-releasable linker comprises a backbone that is stable under physiological conditions (e.g., the backbone is not susceptible to hydrolysis (e.g., aqueous hydrolysis or enzymatic hydrolysis)). In some embodiments, a bispecific adapter that comprises a non-releasable linker does not release any component of the bispecific adapter (e.g., a cancer-targeting ligand or a CAR T-cell targeting ligand). In some embodiments, the non-releasable linker lacks a disulfide bond (e.g., S—S) or an ester in the backbone. In some embodiments, the bispecific adapters comprises a cancer-targeting ligand or a CAR-T cell targeting ligand connected by a backbone that is substantially stable for the entire duration of the bispecific adapter's circulation (e.g., during endocytosis into the target cell endosome). The non-releasable linker can comprise: an amide, ester, ether, amine, and/or thioether (e.g., thio-maleimide). While specific examples are provided, it will be understood that any molecule(s) can be used in the non-releasable linker provided that at least one bond that is not easily or quickly broken under physiological conditions is formed.

Perhaps more specifically, a non-releasable linker can comprise a linker that, at a neutral pH, for example, less than ten percent (10%) (e.g., less than 5%, less than 4%, less than 3%, less than 2%, less than 1%, less than 0.1%, less than 0.01%, or less than 0.001%) will hydrolyze in an aqueous (e.g., buffered (e.g., phosphate buffer)) solution within a period of time (e.g., 24 hours). In some embodiments, where a non-releasable linker is employed, less than about ten percent (10%), and preferably less than five percent (5%) or none, of the bispecific adapter administered releases a moiety to which it is connected (e.g., in systemic circulation prior to uptake by the targeted cells/tissue).

In some embodiments, a cancer-targeting ligand does not cleave from the CAR-T cell targeting ligand of the bispecific adapter in vivo. This may be advantageous as it allows for the bispecific adapter to bind and deliver a CAR-T cell to a targeted cancer cell.

The length of a linker can be selected to optimize linker-imposed separation of molecules on the targeted cell surface, which in turn can facilitate uptake of a bound CAR T-cell into the targeted cell (e.g., when the bispecific adapter is administered). The linker can have a chain length of at least about 5 nm. In certain embodiments, each linker is approximately 5 nm to 15 nm in length. In some embodiments, the linker is at least about 7 nm in length. In certain embodiments, each linker is approximately 7 nm in length and flexible. In certain embodiments, each linker is approximately 7-10 nm in length. In some embodiments, the linker is at least about 14 nm in length. In some embodiments, the linker is about 15 nm in length. In some embodiments, the linker is between about 7 nm and about 31 nm in length (such as, about 7 to 31, 7 to about 31, or 7 to 31), between about 7 nm and about 24 nm in length (such as, about 7 to 24, 7 to about 24, or 7 to 24), or between about 7 nm and about 20 nm in length (such as, about 7 to 20, 7 to about 20, or 7 to 20). In some embodiments, the linker is between about 14 nm and about 31 nm in length (such as, about 14 to 31, 14 to about 31, or 14 to 31), between about 14 nm and about 24 nm in length (such as, about 14 to 24, 14 to about 24, or 14 to 24), or between about 14 nm and about 20 nm in length (such as, about 14 to 20, 14 to about 20, or 14 to 20). In some embodiments, the linker has a chain length of at least 7 nm, at least 14 nm, at least 20 nm, at least 25 nm, at least 30 nm, or at least 40 nm; or from 5 nm to 15 nm, 5 nm to 10 nm, 7 nm to 10 nm, 5 nm to 20 nm, 10 nm to 40 nm, or 25 nm to 100 nm. In certain embodiments, the length of each linker is selected to facilitate micro-clustering of bound molecules on a cell surface to provide at or about 7-10 nm of separation therebetween (such as about 7 nm to about 10 nm, 7 nm to about 10 nm, about 7 nm to 10 nm, or 7 nm to 10 nm). The ranges specified in this paragraph are inclusive of the stated end points and all 1 nm increments encompassed within the stated ranges.

A linker can comprise at least one carbon-carbon bond and/or at least one amide bond.

The linker can comprise one or more L- or D-configurations, natural or unnatural amino acids, or a combination of any of the foregoing.

In certain embodiments, a linker is a group comprising one or more covalently connected structural units.

The linker can be engineered to optimize biodistribution, bioavailability, and PK/PD (e.g., of the bispecific adapter) and/or to increase uptake (e.g., of a CAR-T cell connected thereto and/or the bispecific adapter itself) as previously described into the targeted tissue pursuant to methodologies commonly known in the art or hereinafter developed such as through PEGlaytion and the like.

In some embodiments, linkers can comprise one or more spacers (e.g., to facilitate a particular release time, facilitate an increase in uptake into a targeted tissue, and/or optimize biodistribution, bioavailability, and/or PK/PD of a bispecific adapter provided herein). A spacer may comprise one or more alkyl chains, PEGs, peptides, sugars, peptidoglycans, clickable linkers (e.g., triazoles), rigid linkers such as poly prolines and poly piperidines, and the like.

In some embodiments, a linker of the bispecific adapter comprises PEG, a PEG derivative, or any other linker known in the art or hereinafter developed that can achieve the purpose set forth herein. In some embodiments, the linker is repeated n times, where n is a positive integer. For example, and without limitation, n may be any integer selected from a range of 1-16, 1-32, 1-64, or 1-96. The number of repeats in the linker (i.e., n) can be selected to achieve the desired functionality, size, and/or potency of the conjugate and/or in view of the desired application. In some embodiments, the linker comprises one or more spacers (e.g., which may also be used to specifically design characteristics of the bispecific adapter).

In certain embodiments, the linker comprises, consists of, or consists essentially of PEG₁-PEG₉. In certain embodiments, the linker comprises, consists of, or consists essentially of PEG₂-PEG₈. In certain embodiments, the linker comprises, consists of, or consists essentially of PEG₃-PEG₉. In certain embodiments, the linker comprises, consists of, or consists essentially of PEG₄-PEGs. In certain embodiments, the linker comprises, consists of, or consists essentially of PEG₅-PEG₇. In certain embodiments, the linker comprises, consists of, or consists essentially of PEG₃-PEGis. In certain embodiments, the linker comprises, consists of, or consists essentially of PEG₄-PEG₁₇. In certain embodiments, the linker comprises, consists of, or consists essentially of PEG₅-PEG₁₆. In certain embodiments, the linker comprises, consists of, or consists essentially of PEG₆-PEG₁₅. In certain embodiments, the linker comprises, consists of, or consists essentially of PEG₇-PEG₁₄. In certain embodiments, the linker comprises, consists of, or consists essentially of PEG₈-PEG₁₃. In certain embodiments, the linker comprises, consists of, or consists essentially of PEG₉-PEG₁₂. In certain embodiments, the linker comprises, consists of, or consists essentially of PEG₁₀-PEG₁. The linker can comprise (or consist essentially of or consist of) PEG₃ to PEG₉, such as PEG₃, PEG₄, PEG₅, PEG₆, PEG₇, PEG₈, or PEG₉. The linker can comprise (or consist essentially of or consist of) PEG₆. The linker can comprise an alkyl. The linker can be, comprise, or consist essentially of (CH₂)₄. All ranges stated in this paragraph are inclusive of the stated end points.

The linker can be or comprise (or consist essentially of or consist of) PEG₁. The linker can be or comprise (or consist essentially of or consist of) PEG₆. In certain embodiments, the linker comprises, consists of, or consists essentially of PEG₃. In certain embodiments, the linker comprises, consists of, or consists essentially of PEG₄. In certain embodiments, the linker comprises, consists of, or consists essentially of PEG₁₂. In certain embodiments, the linker comprises, consists of, or consists essentially of PEG₁₆.

In some embodiments, the linker is a hydrolyzable linker. In some embodiments, the linker is a non-hydrolyzable linker. In some embodiments, the linker is an optionally substituted heteroalkyl. In some embodiments, the linker is a substituted heteroalkyl comprising at least one substituent selected from the group consisting of alkyl, hydroxyl, oxo, PEG, carboxylate, and halo. In some embodiments, the linker comprises a spacer (e.g., as described elsewhere herein).

In some embodiments, the linker is substituted heteroalkyl with at least one disulfide bond in the backbone thereof. In some embodiments, the linker is a peptide with at least one disulfide bond in the backbone thereof.

In some embodiments, the linker comprises —CONH—CH(COOH)—CH₂—S—S—CH₂—CRₐR_{b}—O—CO—, —CONH—CH(COOH)CRₐR_{b}—O—CO—, —C(O)NHCH(COOH)(CH₂)₂—CONH—CH(COOH)CRₐR_{b}—O—CO— or —C(O)NHCH(COOH)(CH₂)₂—CONH—CH(COOH)—CH₂—S—S—CH₂—CRₐR_{b}—O—CO—, wherein Rₐ and R_{b} are independently H, alkyl, or heteroalkyl (e.g., PEG).

In some embodiments, the linker comprises a structure of:

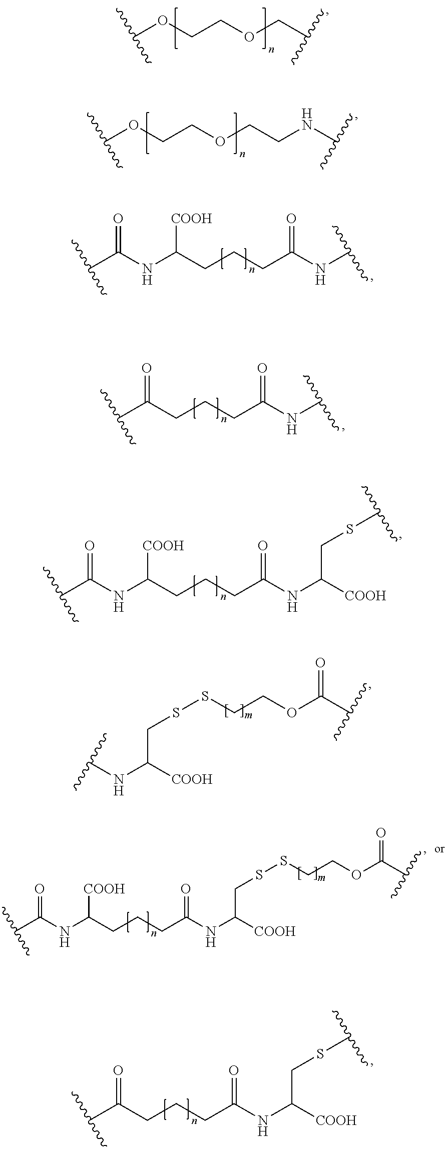

wherein n or m (where applicable) is 0 to 10.

In some embodiments, the linker comprises a structure of

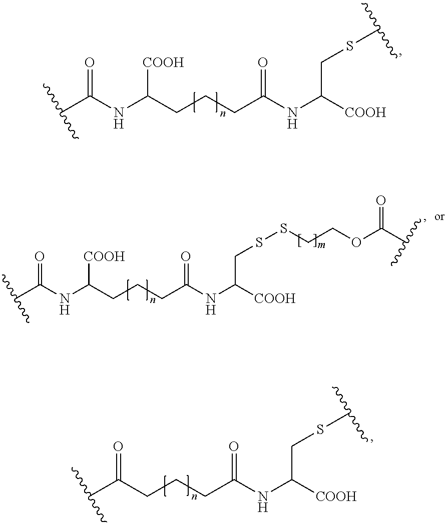

wherein n and m are each independently 0 to 10.

In some embodiments, the linker comprises a structure of:

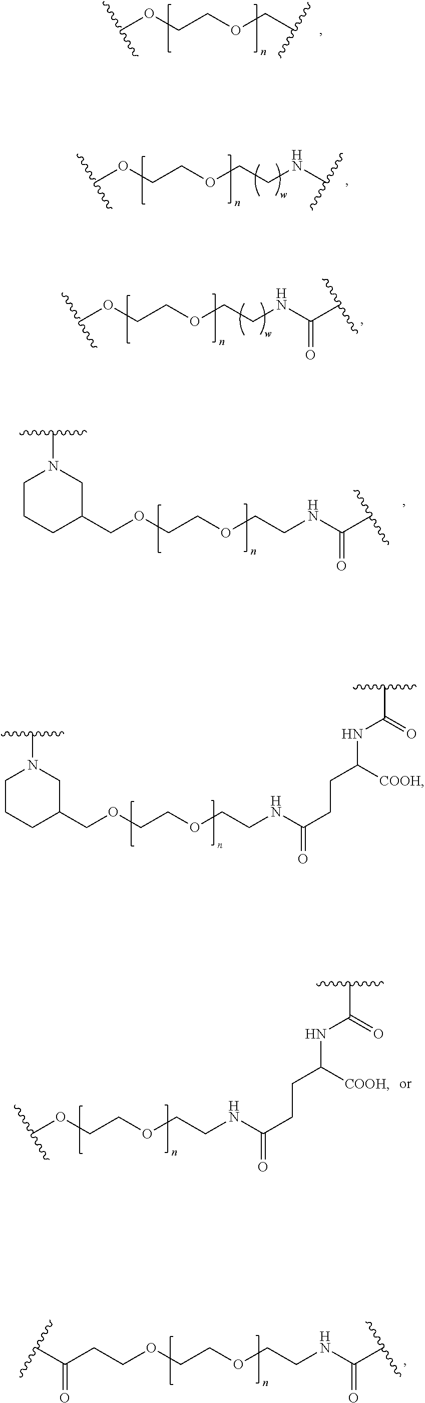

wherein n is 1 to 32. In at least one exemplary embodiment, n is 1 to 30 and w is 0 to 5 (where applicable).

In some embodiments, the linker comprises the structure of:

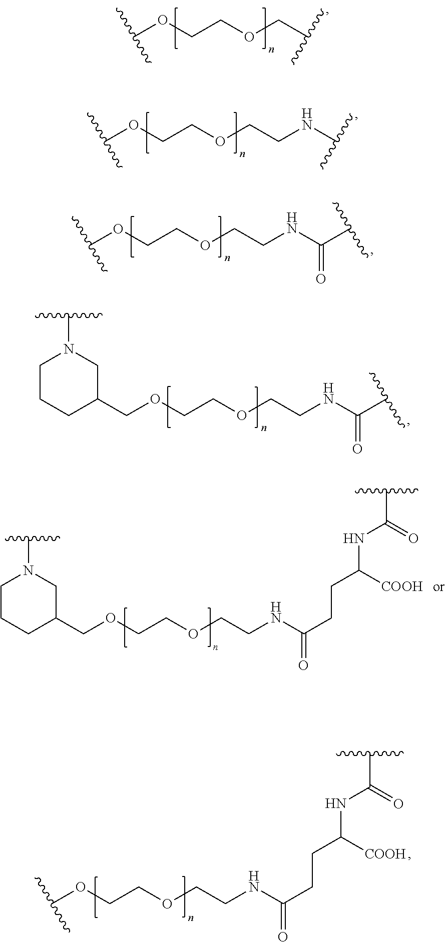

wherein n is 1 to 16.

In certain embodiments, the linker can comprise the structure of:

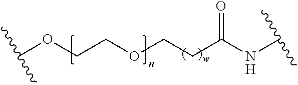

wherein n is 1 to 30 and w is 0 to 5.

### Bispecific Adapters

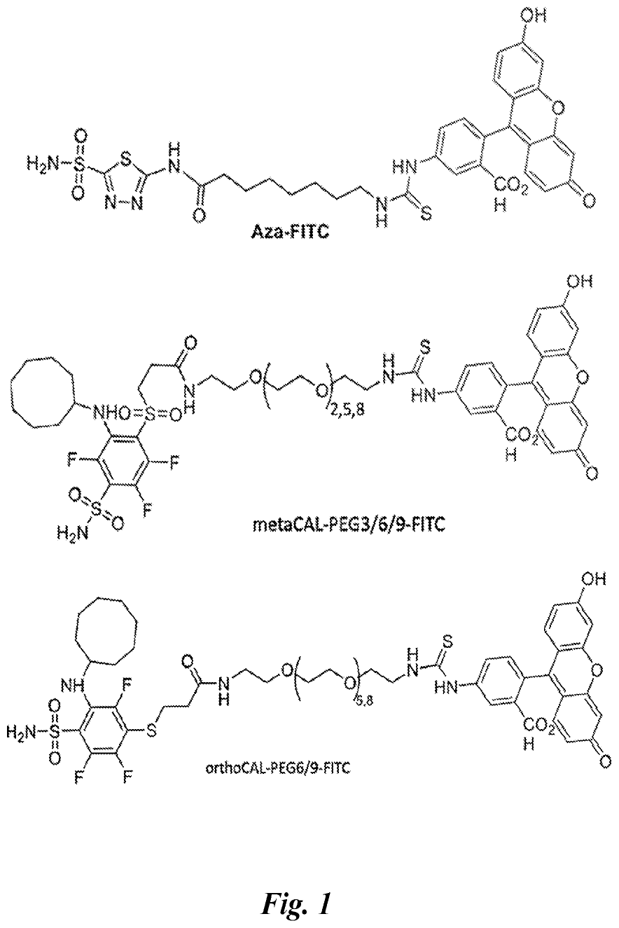

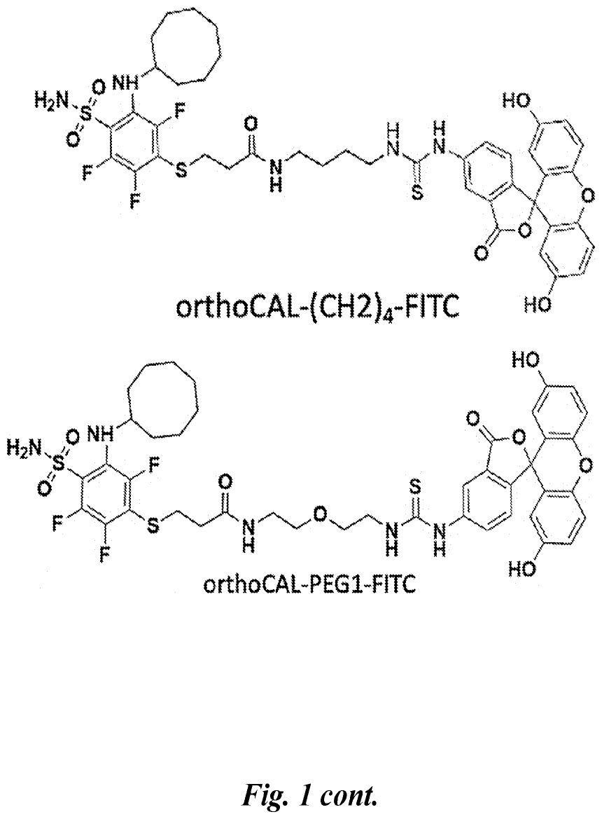

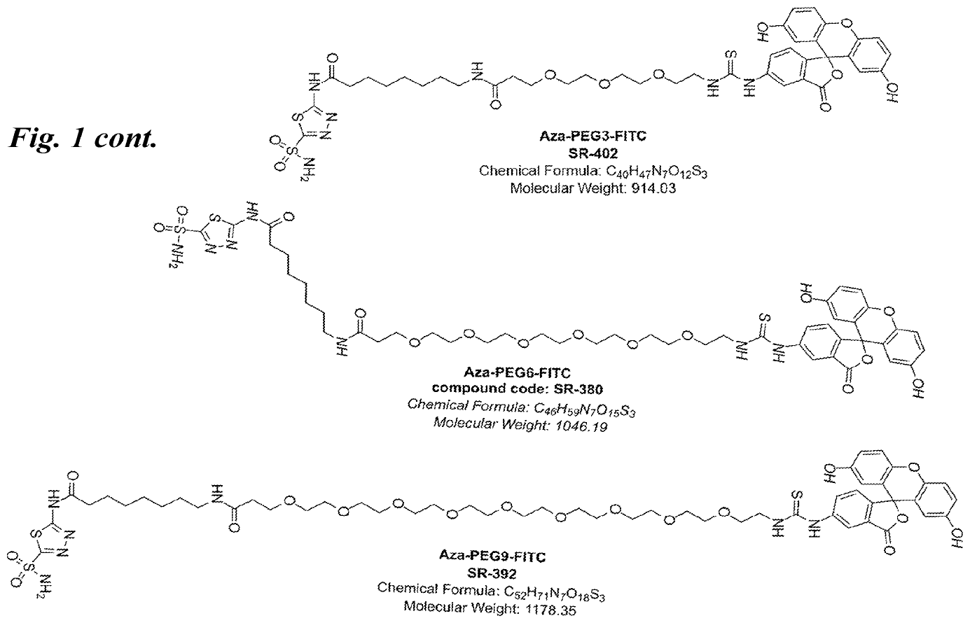

The bispecific adapter, or pharmaceutically acceptable salt or hydrate thereof, can be for use with an anti-fluorescein CAR-T cell in the treatment of a CAIX-expressing cancer and comprise a fluorescein-linker-CAIX ligand. The bispecific adapter can have a structure of the formulae shown in FIG. 1.

The bispecific adapter can have the structure of the following formulae:

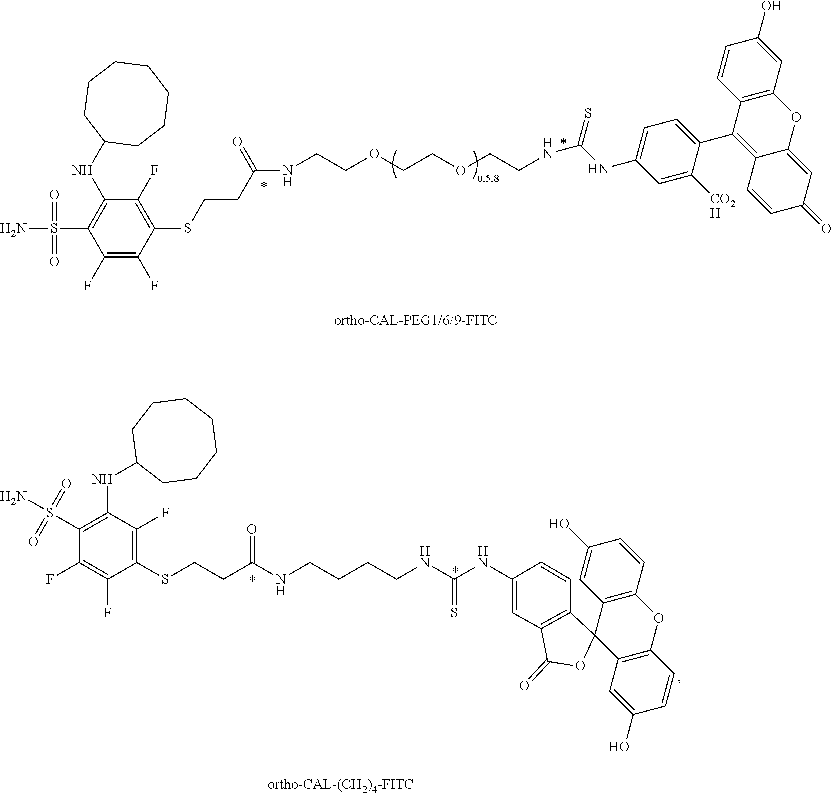

or can be a pharmaceutically acceptable salt or hydrate of either of the foregoing structures.

The bispecific adapter can have the structure of the following formula or be a pharmaceutically acceptable salt or hydrate thereof:

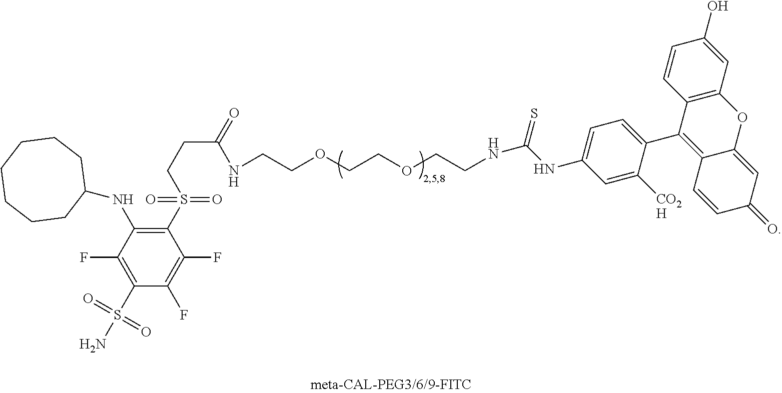

In certain embodiments, the bispecific adapter is for use with an anti-fluorescein CAR-T cell in the treatment of a CAIX-expressing cancer, wherein the fluorescein thereof comprises FITC, the radical of the CAIX ligand is ortho-CAL, and the linker comprises (or consists essentially of or consists of) PEG, or wherein the adapter is a pharmaceutically acceptable salt or hydrate of the foregoing.

In certain embodiments, the bispecific adapter is for use with an anti-fluorescein CAR-T cell in the treatment of a CAIX-expressing cancer, wherein the fluorescein thereof comprises FITC, the radical of the CAIX ligand is ortho-CAL, and the linker comprises (or consists essentially of or consists of) PEG₁, PEG₆, or PEG₉, or wherein the adapter is a pharmaceutically acceptable salt or hydrate of the foregoing.

In certain embodiments, the bispecific adapter is for use with an anti-fluorescein CAR-T cell in the treatment of a CAIX-expressing cancer, wherein the fluorescein thereof comprises FITC, the radical of the CAIX ligand is ortho-CAL, and the linker comprises (or consists essentially of or consists of) (CH₂)₄, or wherein the adapter is a pharmaceutically acceptable salt or hydrate of the foregoing.

In certain embodiments, the bispecific adapter is for use with an anti-fluorescein CAR-T cell in the treatment of a CAIX-expressing cancer, wherein the fluorescein thereof comprises FITC, the radical of the CAIX ligand is meta-CAL, and the linker comprises (or consists essentially of or consists of) PEG₃. PEG₆, or PEG₉, or wherein the adapter is a pharmaceutically acceptable salt or hydrate of the foregoing.

A bispecific adapter for use with an anti-fluorescein CAR-T cell in the treatment of a CAIX-expressing cancer is also provided, wherein the fluorescein thereof comprises fluorescein, FITC, or NHS-fluorescein, the radical of the CAIX ligand is Aza, and the linker comprises (or consists essentially of or consists of) PEG, or wherein the adapter is a pharmaceutically acceptable salt or hydrate of the foregoing. There, the linker can comprise (or consist essentially of or consist of) PEG₁ to PEG₉. The linker can comprise (or consist essentially of or consist of) PEG₆. The linker can comprise (or consist essentially of or consist of) PEG₉.

The bispecific adapter can have a structure of the following formulae or be a pharmaceutically acceptable salt or hydrate thereof:

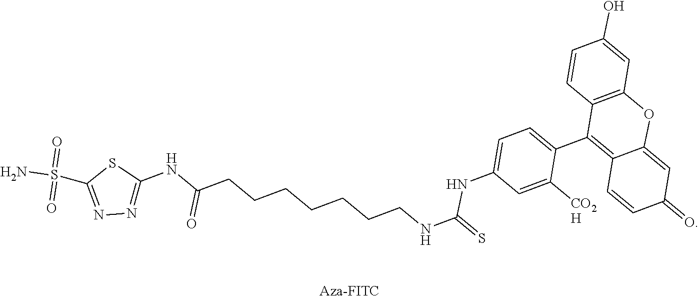 [SMILES: NS(=O)(=O)c1nnc(NC(=O)CCCCCCCNC(=S)Nc2ccc(-c3c4ccc(=O)cc-4oc4cc(O)ccc34)c(C(=O)O)c2)s1]

The bispecific adapter can have a structure of the following formulae, or comprise a pharmaceutically acceptable salt or hydrate thereof:

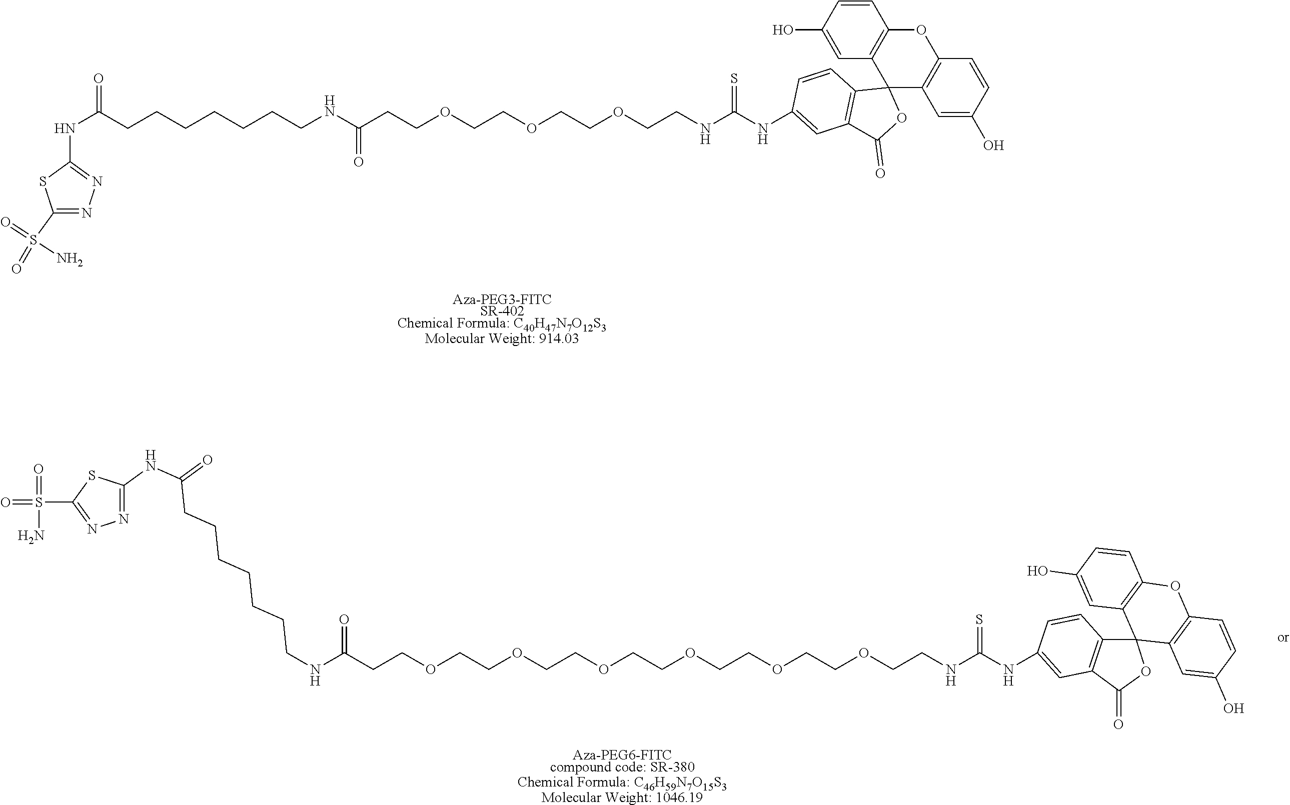

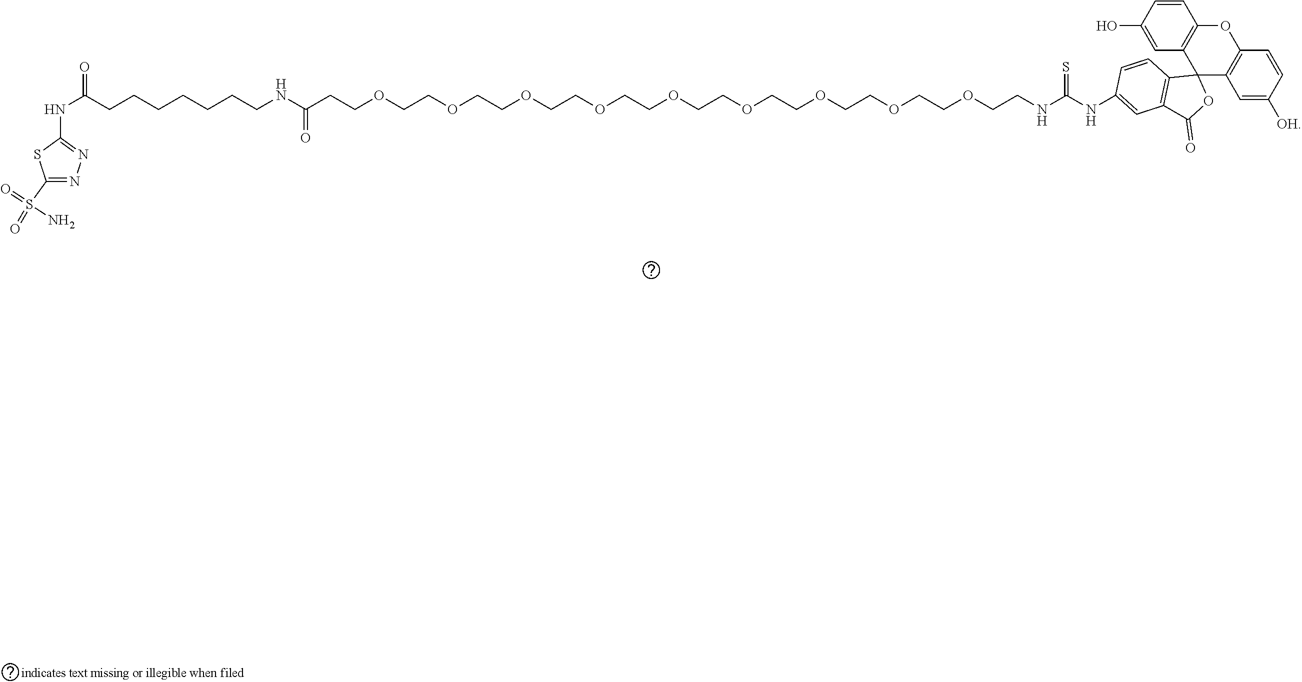 [SMILES: NS(=O)(=O)c1nnc(NC(=O)CCCCCCCNC(=O)CCOCCOCCOCCOCCOCCOCCOCCOCCOCCNC(=S)Nc2ccc3c(c2)C(=O)OC32c3cc(O)ccc3Oc3ccc(O)cc32)s1]

The bispecific adapter may contain one or more chiral centers or may otherwise be capable of existing as multiple stereoisomers. Accordingly, various embodiments of the bispecific adapter may include pure stereoisomers, as well as mixtures of stereoisomers, such as enantiomers, diastereomers, and enantiomerically or diastereomerically enriched mixtures. The bispecific adapter can be capable of existing as geometric isomers, such as pure geometric isomers or mixtures of geometric isomers.

The bispecific adapter conjugates can be synthesized in accordance with methods known in the art. Various methods of synthesis are exemplified in the Examples.

### Salts

The bispecific adapters hereof can be presented as a pharmaceutically acceptable salt. A “pharmaceutically acceptable salt” of a bispecific adapter refers to those salts whose counter ions can be used in pharmaceuticals. Such salts include (i) acid addition salts, which can be obtained by reaction of the free base of the parent conjugate with inorganic acids, such as hydrochloric acid, hydrobromic acid, nitric acid, phosphoric acid, sulfuric acid, perchloric acid, and the like, or with organic acids, such as acetic acid, oxalic acid. (D) or (L) malic acid, maleic acid, methane sulfonic acid, ethane sulfonic acid, p-toluene sulfonic acid, salicylic acid, tartaric acid, citric acid, succinic acid, malonic acid, and the like, and (ii) salts formed when an acidic proton present in the parent conjugate either is replaced by a metal ion, e.g., an alkali metal ion, an alkaline earth ion, or an aluminum ion, or coordinates with an organic base, such as ethanolamine, diethanolamine, triethanolamine, trimethamine, N-methyl glucamine, and the like. Pharmaceutically acceptable salts are well-known to those skilled in the art, and any such pharmaceutically acceptable salt is contemplated herein.

In various embodiments, suitable basic salts are formed from bases which form non-toxic salts. Illustrative examples include arginine, benzathine, calcium, choline, diethylamine, diolamine, glycine, lysine, magnesium, meglumine, olamine, potassium, sodium, tromethamine, and zinc salts. Hemisalts of acids and bases also may be formed, e.g., hemisulphate and hemicalcium salts.

Pharmaceutically acceptable salts can be synthesized from the parent bispecific adapter conjugate which contains a basic or acidic moiety by conventional chemical methods. In some instances, such salts can be prepared by reacting the free acid or base forms of these conjugates with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The bispecific adapter, or pharmaceutically acceptable salt or hydrate thereof, may exist in unsolved forms as well as solvated forms, including hydrated forms. Solvated forms can be equivalent to unsolvated forms. In each embodiment hereof, it will be understood that the formulae include and represent not only all pharmaceutically acceptable salts of the bispecific adapters, but also include any and all hydrates and/or solvates of the conjugate formulae or salts thereof. The term “solvate” means a compound, or a salt thereof, that further includes a stoichiometric or non-stoichiometric amount of solvent bound by non-covalent intermolecular forces. Where the solvent is water, the solvate is a hydrate.

Certain functional groups, such as the hydroxy, amino, and like, can form complexes and/or coordination conjugates with water and/or various solvents. Accordingly, the formulae are to be understood to include and represent those various hydrates and/or solvates. Non-hydrates and/or non-solvates of the bispecific adapters are also included.

### Pharmaceutical Compositions

In view of the above, also provided is a composition (e.g., a pharmaceutical composition) for the treatment of cancer (e.g., a CAIX-expressing cancer) comprising at least one bispecific adapter and a pharmaceutically acceptable carrier or excipient. “Pharmaceutically acceptable carrier” includes any of the standard pharmaceutical carriers, such as, but not limited to, a buffering agent, a preserving agent, an anesthetic agent, a solubilizing agent, an isotonic agent, a wetting agent, and a stabilizer. The term also encompasses any of the agents approved by a regulatory agency, such as the U.S. Food and Drug Administration, or listed in the U.S. Pharmacopeia for use in animals (e.g., mammals, such as humans). The carrier can be a phosphate-buffered saline solution, water, or an emulsion such as an oil/water or water/oil emulsion.

Also provided is a pharmaceutical composition for use in the treatment of a CAIX-expressing cancer comprising any of the bispecific adapters hereof (e.g., a fluorescein-linker-CAIX) and a pharmaceutically acceptable carrier or excipient.

The bispecific adapters can be formulated as pharmaceutical compositions and administered to a mammalian host, such as a human patient, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intrasternal, intracranial, intratumoral, intramuscular, topical, inhalation and/or subcutaneous routes. Indeed, in at least one embodiment, a bispecific adapter and/or composition as described herein can be administered directly into the blood stream, into muscle, or into an internal organ.

For example, in at least one embodiment, the present bispecific adapters can be systemically administered (orally, for example) in combination with a pharmaceutically acceptable vehicle such as an inert diluent or an assimilable edible carrier. For oral therapeutic administration, the bispecific adapter can be combined with one or more excipients and used in the form of ingestible tablets, buccal tablets, troches, capsules, elixirs, suspensions, syrups, wafers, and the like. The percentage of the compositions and preparations may vary and may be between about 1 to about 99% weight of the active ingredient(s) and a binder, excipients, a disintegrating agent, a lubricant, and/or a sweetening agent (as are known in the art). The amount of active conjugate in such therapeutically useful compositions is such that an effective dosage level will be obtained.

The bispecific adapters and pharmaceutical compositions hereof can be formulated as parenteral formulations. Parenteral formulations are typically aqueous solutions, which can contain carriers or excipients such as salts, carbohydrates, and buffering agents (preferably at a pH of from 3 to 9), but they can be more suitably formulated as a sterile, non-aqueous solution or as a dried from to be used in conjunction with a suitable vehicle such as sterile, pyrogen-free water or sterile saline. Preparation under sterile conditions, by lyophilization to produce a sterile, lyophilized powder for a parenteral formulation, can be accomplished using methods well-known in the art. The solubility of the bispecific adapter, or a pharmaceutically acceptable salt or hydrate thereof, for parenteral formulation can be increased by the use of appropriate formulation techniques, such as the incorporation of solubility-enhancing agents.

The bispecific adapters/compositions can also be administered via infusion or injection (e.g., using needle (including microneedle) injectors and/or needle-free injectors). Solutions of the composition can be aqueous, optionally mixed with a nontoxic surfactant and/or can contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water or phosphate-buffered saline (PBS). For example, dispersions can be prepared in glycerol, liquid PEGs, triacetin, and mixtures thereof and in oils. Under ordinary conditions of storage and use, these preparations can further contain a preservative to prevent the growth of microorganisms.

The pharmaceutical dosage forms suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants. The action of microorganisms can be prevented by the addition of various antibacterial and antifungal agents such as parabens, chlorobutanol, phenol, sorbic acid, thimerosal, and the like. In certain cases, it will be desirable to include one or more isotonic agents such as sugars, buffers, or sodium chloride. Prolonged absorption of the injectable compositions can be brought about by the incorporation of agents formulated to delay absorption, for example, aluminum monostearate and gelatin.

Sterile injectable solutions can be prepared by incorporating the bispecific adapter(s) and/or composition in the required amount of the appropriate solvent with one or more of the other ingredients set forth above, as required, followed by filter sterilization. In the case of sterile powders for the preparation of sterile injectable solutions, the preferred methods of preparations are vacuum drying and freeze drying techniques, which yield a powder of the active ingredient plus any additional desired ingredient present in the previously sterile-filtered solutions.

For topical administration, it may be desirable to administer the bispecific adapters to the skin as compositions or formulations in combination with a dermatologically acceptable carrier, which may be a solid or a liquid. For example, in certain embodiments, solid carriers may include finely divided solids such as talc, clay, microcrystalline cellulose, silica, alumina and the like. Similarly, useful liquid carriers may comprise water, alcohols or glycols or water-alcohol/glycol blends, in which the present conjugates can be dissolved or dispersed at effective levels, optionally with the aid of non-toxic surfactants. Additionally or alternatively, adjuvants such as fragrances and antimicrobial agents can be added to optimize the properties for a given use. The resultant liquid compositions can be applied from absorbent pads, used to impregnate bandages and/or other dressings, sprayed onto the targeted area using pump-type or aerosol sprayers, or simply applied directly to a desired area of the subject.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like for application directly to the skin of the subject.

The amount of the bispecific adapter (or pharmaceutically acceptable salt or hydrate thereof) to be administered to a subject can vary significantly, depending on the cancer being treated, the route of administration, and tissue distribution. As used herein, the terms “therapeutically effective,” “therapeutically effective dose,” “therapeutically effective amount,” “prophylactically effective amount,” or “prophylactically effective dose” mean (unless specifically stated otherwise) a quantity of a bispecific adapter which, when administered either one time or over the course of a treatment cycle affects the health, wellbeing or mortality of a subject (e.g., and without limitation, delays the onset of and/or reduces the severity of one or more of the symptoms associated with a cancer). Useful dosages of the bispecific adapters can be determined by comparing their in vitro activity, and the in vivo activity in animal models. Methods of the extrapolation of effective dosages in mice and other animals to human subjects are known in the art. Indeed, the dosage of the bispecific adapter can vary significantly depending on the condition of the host subject, the cancer being treated, how advanced the pathology is, the route of administration of the bispecific adapter and tissue distribution, and the possibility of co-usage of other therapeutic treatments (such as radiation therapy or additional drugs in combination therapies such as, for example CAR T-cell therapy). The amount of the composition required for use in treatment (e.g., the therapeutically or prophylactically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise.

The amount to be administered to a subject can range, for example, from about 0.05 mg to about 30 mg, about 0.05 mg to about 25 mg, about 0.05 mg to about 20 mg, about 0.05 mg to about 15 mg, about 0.05 mg to about 10 mg, about 0.05 mg to about 9 mg, about 0.05 mg to about 8 mg, about 0.05 mg to about 7 mg, about 0.05 mg to about 6 mg, about 0.05 mg to about 5 mg, about 0.05 mg to about 4 mg, about 0.05 mg to about 3 mg, about 0.05 mg to about 2 mg, about 0.05 mg to about 1 mg, about 0.05 mg to about 0.5 mg, about 0.05 mg to about 0.4 mg, about 0.05 mg to about 0.3 mg, about 0.05 mg to about 0.2 mg, about 0.05 mg to about 0.1 mg, about 0.01 mg to about 20 mg, about 0.3 mg to about 10 mg, about 0.1 mg to about 20 mg, or about 0.8 mg to about 3 mg. The ordinarily skilled artisan will readily appreciate that the dose may vary within the various ranges provided above based on the factors pointed out above and may be at the treating physician's discretion.

Therapeutically effective or prophylactically effective amounts or doses can range, for example, from about 0.05 mg/kg of patient body weight to about 30.0 mg/kg of patient body weight, or from about 0.01 mg/kg of patient body weight to about 5.0 mg/kg of patient body weight, including but not limited to 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg. 1.0 mg/kg, 1.5 mg/kg, 2.0 mg/kg, 2.5 mg/kg. 3.0 mg/kg, 3.5 mg/kg, 4.0 mg/kg, 4.5 mg/kg, and 5.0 mg/kg, all of which are kg of patient body weight. The total therapeutically or prophylactically effective amount of the bispecific adaptor can be administered in single or divided doses and may, at the practitioner's discretion, fall outside of the typical range given herein.

In another embodiment, the bispecific adaptor can be administered in a therapeutically or prophylactically effective amount of from about 0.5 g/m² to about 500 mg/m², from about 0.5 g/m² to about 300 mg/m², or from about 100 g/m² to about 200 mg/m². In other embodiments, the amounts can be from about 0.5 mg/m² to about 500 mg/m², from about 0.5 mg/m² to about 300 mg/m², from about 0.5 mg/m² to about 200 mg/m², from about 0.5 mg/m² to about 100 mg/m², from about 0.5 mg/m² to about 50 mg/m², from about 0.5 mg/m² to about 600 mg/m², from about 0.5 mg/m² to about 6.0 mg/m², from about 0.5 mg/m² to about 4.0 mg/m², or from about 0.5 mg/m² to about 2.0 mg/m². The total amount can be administered in single or divided doses and may, at the physician's discretion, fall outside of the typical range given herein. These amounts are based on meters of body surface area. All ranges specified in this paragraph are inclusive of the stated end points and include all 0.5 g/m² increments encompassed in each specified range.

In other embodiments, the amount of the bispecific adapter (or pharmaceutically acceptable salt or hydrate thereof) to be administered to a subject can range, for example, from about 50 nmol/kg to about 3,000 nmol/kg of subject body weight, about 50 nmol/kg to about 2,000 nmol/kg, about 50 nmol/kg to about 1,000 nmol/kg, about 50 nmol/kg to about 900 nmol/kg, about 50 nmol/kg to about 800 nmol/kg, about 50 nmol/kg to about 700 nmol/kg, about 50 nmol/kg to about 600 nmol/kg, about 50 nmol/kg to about 500 nmol/kg, about 50 nmol/kg to about 400 nmol/kg, about 50 nmol/kg to about 300 nmol/kg, about 50 nmol/kg to about 200 nmol/kg, about 50 nmol/kg to about 100 nmol/kg, about 100 nmol/kg to about 300 nmol/kg, about 100 nmol/kg to about 500 nmol/kg, about 100 nmol/kg to about 1,000 nmol/kg, or about 100 nmol/kg to about 2,000 nmol/kg of subject body weight. In other embodiments, the dose can be about 100 nmol/kg, about 150 nmol/kg, about 200 nmol/kg, about 250 nmol/kg, about 300 nmol/kg, about 350 nmol/kg, about 400 nmol/kg, about 450 nmol/kg, about 500 nmol/kg, about 600 nmol/kg, about 700 nmol/kg, about 800 nmol/kg, about 900 nmol/kg, about 1,000 nmol/kg, about 2,000 nmol/kg, or about 3,000 nmol/kg of subject body weight. In other embodiments, between about 20 g/kg to about 3 mg/kg of subject body weight can be administered. The amount can be between about 0.2 mg/kg to about 0.4 mg/kg of subject body weight or about 50 μg/kg subject body weight. All ranges specified in this paragraph are inclusive of the stated end points and include all 1 nmol/kg or 10 g/kg increments, as applicable, encompassed in each specified range.

### Uses and Methods

Further provided is a method of treating cancer in a subject (e.g., a CAIX-expressing cancer). The method comprises administering to the subject cancer-treatment effective amounts of (i) anti-fluorescein (e.g., fluorescein, FITC, or NHS-fluorescein) CAR-T cells or a pharmaceutical composition comprising the same and a pharmaceutically acceptable carrier or excipient, and (ii) a bispecific adapter or a pharmaceutical composition comprising the same and a pharmaceutically acceptable carrier or excipient.

The terms “treat,” “treating,” “treated,” and “treatment” refer to therapeutic treatment. Such treatment can have a prophylactic effect. Cancer is treated when the symptoms or signs of cancer are ameliorated, such as a reduction in the size of a tumor, complete or partial elimination of a tumor, stabilization of cancer such as by inhibiting the progression of cancer (e.g., increase in the size of a tumor or increase in the number of tumors, such as due to metastasis), or any other effect on the cancer that a physician would consider to constitute therapeutic (or prophylactic) treatment.

The term “subject,” as used herein, means an animal, such as a mammal, and in particular a human. In veterinary applications, the subject can be a laboratory, an agricultural, a domestic, or a wild animal. Examples of such animals include, but are not limited to, a rodent, a rabbit, a monkey, a chimpanzee, a dog, a cat, a cow, a horse, a pig, a sheep, a goat, a bear, a panda, a lion, a tiger, a leopard, an elephant, a zebra, a giraffe, a gorilla, a dolphin, or a whale.

The anti-fluorescein (e.g., fluorescein, FITC, or NHS-fluorescein) CAR-T cells are T cells (alternatively, NK cells can be used) engineered to express a CAR that recognizes and binds to fluorescein (e.g., fluorescein, FITC, or NHS-fluorescein) in the bispecific adapter.

The CAR is a fusion protein comprising at least three domains, which include (i) a recognition region (e.g., a single-chain fragment variable (scFv) region of an antibody), which recognizes and binds to fluorescein (e.g., fluorescein, FITC, or NHS-fluorescein) with specificity, (ii) a co-stimulation domain, which enhances the proliferation and survival of the T lymphocytes, and (iii) an activation signaling domain, which generates a cytotoxic T lymphocyte activation signal.

scFv regions of antibodies that bind fluorescein (e.g., in FITC) can be used and prepared from (i) an antibody known in the art that binds to fluorescein (e.g., fluorescein, FITC, or NHS-fluorescein), (ii) a newly prepared anti-fluorescein antibody, or (iii) sequence variants derived from the scFv regions of such antibodies, e.g., scFv regions having at least about 80%, at least about 90%, at least about 95%, at least about 97%, at least about 98%, at least about 99%, or at least about 99.5% sequence identity to the amino acid sequence of the scFv region from which they are derived. The binding portion of the CAR can be, for example, an scFv of an antibody, an Fab, Fv, Fc, or (Fab′)₂ fragment.

“Percent (%) sequence identity” with reference to a polypeptide or nucleotide sequence is defined as the percentage of amino acid or nucleic acid residues, respectively, in a candidate sequence that are identical with the residues in the reference sequence, after aligning the sequences and introducing gaps, if necessary, to achieve the maximum percent sequence identity and not considering any conservative substitutions as part of the sequence identity. Alignment for purposes of determining percent sequence identity can be achieved in various ways that are within the skill of the art, for instance, using publicly available computer software. For example, determination of percent identity or similarity between sequences can be done, for example, by using the GAP program (Genetics Computer Group, software; now available via Accelrys online), and alignments can be done using, for example, the ClustalW algorithm (VNTI software, InforMax Inc.). Further, a sequence database can be searched using the nucleic acid or amino acid sequence of interest. Algorithms for database searching are typically based on the BLAST software (Altschul et al., 1990), but those skilled in the art can determine appropriate parameters for aligning sequences, including any algorithms needed to achieve maximal alignment over the full length of the sequences being compared. In some embodiments, the percent identity can be determined along the full-length of the nucleic acid or amino acid sequence.

In various embodiments, the CAR has a recognition region and the recognition region is a scFv region of an anti-fluorescein antibody, which can bind fluorescein, FITC, or NHS-fluorescein (see, e.g., the E2 anti-fluorescein antibody described in Vaughan et al., Nature Biotechnol 14(3): 309-314 (1996), and the exemplary CAR construct, which expresses a CAR comprising the E2 anti-fluorescein antibody, shown in FIG. 1 and described on page 66, line 16, through page 69, line 12, of International Patent Application Publication No. WO 2019/144091, both of which are hereby incorporated by reference for their teachings regarding same). The CAR has a co-stimulation domain, and the co-stimulation domain can be CD28 (cluster of differentiation 28), CD2 (cluster of differentiation 2), CD137 (cluster of differentiation 137; 4-1BB), a member of the tumor necrosis factor (TNF) family, CD134 (cluster of differentiation 134; OX40), a member of the TNF receptor (TNFR) super family of receptors, CD27 (cluster of differentiation 27). CD30 (cluster of differentiation 30), CD150 (cluster of differentiation 150), DAP10, NKG2D, CD278 (cluster of differentiation 278; ICOS), a CD28-superfamily co-stimulatory molecule expressed on activated T cells, signaling lymphocytic activation molecule (SLAM)-related receptor family (such as 2B4), or any combination thereof. Sequence variants of the co-stimulation domains, which have the same or similar activity as the domain on which they are modeled, also can be used without adversely impacting the method. The CAR has an activation signaling domain, and the activation signaling domain can be a T cell CD3ζ chain, CD3 delta receptor protein, mbl receptor protein, B29 receptor protein, or an Fc receptor γ. Sequence variants of the activation signaling domains, which have the same or similar activity as the domain on which they are modeled, also can be used without adversely impacting the method. Such co-stimulation domains and variants of such co-stimulation domains and activation signaling domains can have at least about 80%, at least about 90%, at least about 95%, at least about 97%, at least about 98%, at least about 99%, or at least about 99.5% sequence identity to the amino acid sequence of the domain from which they are derived.

In an embodiment of a CAR comprising an E2 anti-fluorescein antibody fragment, the CAR comprises an IgG4 hinge domain and a CD28 transmembrane domain. The co-stimulation domain is CD137 (4-1BB), and the activation signaling domain is CD3ζ.

In certain embodiments, a CAR comprises an scFv of an anti-fluorescein antibody as a recognition region, a CD137 (4-1BB) co-stimulation domain, and CD3ζ as an activation signaling domain.

Constructs encoding CARs are prepared using genetic engineering techniques. Such techniques are described, for example, in Sambrook et al., Molecular Cloning: A Laboratory Manual, 3^{rd} ed., Cold Spring Harbor Laboratory Press (2001), which is hereby incorporated by reference. By way of example, a plasmid or viral expression vector (e.g., a lentiviral vector, a retroviral vector, sleeping beauty, and piggyback (transposon/transposase systems that include a non-viral-mediated CAR gene delivery system)) can be prepared to encode a fusion protein comprising a recognition region, one or more co-stimulation domains, and an activation signaling domain in frame and linked in a 5′ to 3′ direction. Other arrangements can be acceptable and can include a recognition region, an activation signaling domain, and one or more co-stimulation domains. The placement of the recognition region in the fusion protein will generally be such that display of the region on the exterior of the cell is achieved. The CAR can also include additional elements, such as a signal peptide to ensure proper export of the fusion protein to the cell surface, a transmembrane domain to ensure the fusion protein is maintained as an integral membrane protein, and a hinge domain that imparts flexibility to the recognition region and allows strong binding to the CAR-targeting moiety.

T lymphocytes (e.g., cytotoxic T lymphocytes) can be genetically engineered to express CAR constructs by transfecting a population of the T lymphocytes with an expression vector encoding the CAR construct. Suitable methods for preparing a transduced population of T lymphocytes expressing a selected CAR construct are well-known to the skilled artisan and are described in Sambrook et al. (2001), supra.

The T lymphocytes can be autologous, although heterologous cells can be used, such as when the patient being treated has received high-dose chemotherapy or radiation treatment to destroy the patient's immune system. In various embodiments, allogeneic cells can be used.

T lymphocytes can be obtained from a patient by means well-known in the art. For example, T cells can be obtained by collecting peripheral blood from the patient, subjecting the blood to Ficoll density gradient centrifugation, and then using a negative T cell isolation kit (such as EasySep™ T Cell Isolation Kit) to isolate a population of cytotoxic T cells from the peripheral blood. In various embodiments, the population of cytotoxic T lymphocytes need not be pure and may contain other cells, such as other T cells, monocytes, macrophages, natural killer cells, and B cells. The population of cells being collected can comprise at least about 90% of the selected cell type, such as at least about 91%, 92%, 93%, 94%, 95%, 96%, 97%, 98%, 99% or 100% of the selected cell type.

After the T lymphocytes are obtained, the cells can be cultured under conditions that promote the activation of the cells. The culture conditions can be such that the cells can be administered to a patient without concern for reactivity against components of the culture medium. For example, the culture conditions may not include bovine serum products, such as bovine serum albumin (BSA). Activation can be achieved by introducing known activators into the culture medium, such as anti-CD3 antibodies in the case of cytotoxic T cells. Other suitable activators include anti-CD28 antibodies. The population of lymphocytes can be cultured under conditions promoting activation for about 1 to about 4 days. The appropriate level of activation can be determined by cell size, proliferation rate, or activation markers determined by flow cytometry.

After the population of cytotoxic T lymphocytes has been cultured under conditions promoting activation, the cells can be transfected with an expression vector encoding a CAR. After transfection, the cells can be immediately administered to the patient or the cells can be cultured for at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days, for example, to allow time for the cells to recover from the transfection. Suitable culture conditions can be similar to the conditions under which the cells were cultured for activation, either with or without the agent that was used to promote activation.

When the cells have been transfected and activated, a composition comprising the CAR-T cells can be prepared and administered to the subject. Culture media that lack any animal products, such as BSA, can be used. Tissue culture conditions typically used in the art can be used to avoid contamination with bacteria, fungi, and mycoplasma. Cells can be pelleted, washed, and resuspended in a pharmaceutically acceptable carrier, diluent, or excipient.

Exemplary compositions comprising CAR-T cells include compositions comprising the cells in sterile 290 mOsm saline, infusible cryomedia (containing Plasma-Lyte A, dextrose, sodium chloride, human serum albumin (HSA), and dimethylsulfoxide (DMSO)) in 0.9% NaCl with 2% HSA, or in any other sterile 290 mOsm infusible material. Depending on the identity of the culture medium, the CAR-T cells can be administered in the culture medium as the composition or concentrated and resuspended in the culture medium before administration.

The CAR-T cell composition can be administered to the subject by any suitable means, such as parenteral administration, e.g., intradermally, subcutaneously, intramuscularly, intraperitoneally, intravenously, or intrathecally.

The total number of CAR-T cells and the concentration of the cells in the composition administered to the subject will vary depending on a number of factors including the type of CAR-T cells being used, the binding specificity of the CAR, the identity of the CAR-targeting moiety (in the examples herein. FITC), and the identity small molecule ligand/targeting ligand of the bispecific adapter (e.g., a CAIX ligand), the identity of the cancer, the location of the cancer in the subject, the means used to administer the compositions to the subject, and the health, age and weight of the subject being treated. Suitable compositions comprising transduced CAR-T cells include those having a volume of between about 5 ml and about 200 ml, containing from about 1×10⁵ to about 1×10¹⁵ transduced CAR-T cells. Typical compositions comprise a volume of between about 10 ml and about 125 ml and contain from about 1×10⁷ to about 1×10¹⁰ CAR-T cells. An exemplary composition comprises about 1×10⁹ CAR-T cells in a volume of about 100 ml. A single dose or multiple doses of the CAR-T cells can be administered to the subject. Compositions can comprise about 1 million (M), 2M, 3M, 4M, 5M, 6M, 7M, 8M, 9M, 10 M, 11 M, 12M, 12.5M, 13M, 14M or 15M CAR-T cells, such as per kg of patient body weight. When the CAR-T cell composition is administered by injection into the subject's bloodstream, the CAR-T cells in the subject's bloodstream are at least 5%, 7%, 10%, 11%, 12%, 13%, 14%, or 15% of the subject's total T cells in the subject's bloodstream by about four weeks after injection, at least 20%, 25%, 30%, 35%, 40%, or 50% of the subject's total T cells in the subject's bloodstream by about two weeks after injection, or at least 85%, 90% or 95% of the subject's total T cells by about one week after injection.

The bispecific adapter (or pharmaceutically acceptable salt or hydrate thereof) or pharmaceutical composition comprising same, or a combination thereof and the anti-fluorescein CAR-T cells or pharmaceutical composition comprising the anti-fluorescein CAR-T cells can be administered to the patient using any suitable method known in the art. The terms “administer,” “administering,” “administered.” and “administration” refer to methods of introducing the bispecific adapter (or a pharmaceutically acceptable salt or hydrate thereof) or a pharmaceutical composition comprising the bispecific adapter (or a pharmaceutically acceptable salt or hydrate thereof) and methods of introducing the anti-fluorescein CAR-T cells or a pharmaceutical composition comprising the anti-fluorescein CAR-T cells. Examples of suitable routes of administration include, but are not limited to, oral, intravenous, intramuscular, subcutaneous, and transdermal. The components can be administered directly into the blood stream, into muscle, or into an internal organ. Suitable routes for parenteral administration include, but are not limited to, intravenous, intra-arterial, intraperitoneal, intrathecal, epidural, intracerebroventricular, intraurethral, intrasternal, intracranial, intratumoral, intramuscular, and subcutaneous. Use can be made of needle injectors, including microneedles, needle-free injectors, and infusions. The components can be administered in unit dosage forms and/or formulations containing conventional non-toxic pharmaceutically acceptable carriers or excipients (or vehicles or adjuvants).

In the method, the anti-fluorescein CAR-T cells (or pharmaceutical composition comprising anti-fluorescein CAR-T cells and a pharmaceutically acceptable carrier or excipient) and the bispecific adapter can be administered simultaneously or sequentially, in either order, by the same or different routes. When administered simultaneously by the same route, the formulations can be the same or different. In various embodiments, the bispecific adapter can be administered to the subject after the CAR-T cells. The timing between the administration of CAR-T cells and the administration of the bi-specific adapter can vary widely depending on factors that include the type of CAR-T cells being used, the binding specificity of the CAR, the identity of the CAR-targeting moiety (in the examples herein, a fluorescein) and the small molecule ligand/targeting moiety of the bispecific adapter (i.e., CAIX ligands), the identity of the cancer, the location in the subject of the cancer, the means used to administered to the subject the CAR-T cells and the bispecific adapter, as well as the health, age, and weight of the patient.

The bispecific adapter(s) can be administered before or after the CAR-T cells, such as within about 3, 6, 9, 12, 15, 18, 21 or 24 hours, or within about 0.5, 1, 1.5, 2, 2.5, 3, 4, 5, 6, 7, 8, 9, 10 or more days. The rate of tumor lysis can be regulated by adjusting the rate of administration of the bispecific adapter, for example (e.g., as a function of dosing schedule, such as continuous, once daily, twice daily, thrice daily, once weekly, twice weekly, or thrice weekly). By “continuous” is meant for at least one hour, at least four hours, at least six hours, at least eight hours, at least 10 hours, at least 12 hours, or at least 24 hours, or a regimen of daily or weekly administration, such as once/day twice/day, thrice/day, every other day, once/week, twice/week, thrice/week or any other suitable regimen. In the method, the anti-fluorescein CAR-T cells (or pharmaceutical composition comprising anti-fluorescein CAR-T cells and a pharmaceutically acceptable carrier or excipient) and the bispecific adapter can be administered intravenously. The cancer can be ovarian cancer, breast cancer, lung cancer, bladder cancer, or clear cell renal cell carcinoma (e.g., stage 3-4 clear cell renal cell carcinoma). The cancer can be endometrial cancer or glioma (e.g., stage 3-4 glioma).

Cytokine release syndrome (CRS) can be controlled by varying the dose of the bispecific adapter. See, e.g., International Patent Application Publication No. WO 2017/177149.

Such combination therapy methods can be performed using any engineered cell that is suitable for the treatment of cancer and can include using more than one of these types of agents. In certain embodiments, the engineered cell used in this combination therapy are CAR T-cells and can also (or alternatively) comprise engineered stem cells and other cells.

The engineered cells used in combination with the bispecific adapters or compositions can be any CAR T cells, stem cells or other engineered cells or combination thereof. Various adoptive cell therapies (also termed cellular immunotherapy) are known in the art for use in the treatment of cancer, and T-cell immunotherapy, in particular, has received much attention. Some non-limiting examples of such therapies include engineered T cell receptor (TCR) therapy, CAR T cell therapy, and natural killer (NK) cell therapy.

In certain approaches, administering both the bispecific adaptor conjugates and the engineered cellular therapy results in a greater than additive inhibition of growth of the cancer.

Where multiple therapeutics and/or therapies are co-administered, dosages may be adjusted accordingly, as is recognized in the pertinent art. “Co-administration” and combination therapy are not limited to simultaneous administration, but also include treatment regimens in which a targeted bispecific adaptor is administered at least once during a course of treatment that involves administering a cellular therapy to a subject.

The methods of treating cancer hereof can comprise administering any of the bispecific adapters to the patient and administering any of the engineered cell compositions or engineered cell therapy to the patient.

In certain embodiments, a method of treating cancer in a subject is provided. The method comprises administering to the subject cancer-treatment effective amounts of (i) anti-fluorescein CAR-T cells or a pharmaceutical composition comprising anti-fluorescein CAR-T cells and a pharmaceutically acceptable carrier or excipient; and (ii) any bispecific adapter or any pharmaceutical composition comprising the same and a pharmaceutically acceptable carrier or excipient. Steps (i) and (ii) can be administered simultaneously or sequentially, in either order, by the same or different routes.

The anti-fluorescein CAR-T cells can comprise any CAR T-cells suitable for use as described. In certain embodiments, anti-fluorescein CAR-T cells comprise a recognition region comprising a scFv region of an anti-fluorescein antibody; a co-stimulation domain, wherein the co-stimulation domain is CD28, CD137 (4-1BB), CD134 (OX40), or CD278 (ICOS); and/or an activation signaling domain that is a T cell CD3ζ chain or an Fc receptor γ.

In certain embodiments, both steps (i) and (ii) of the method are administered intravenously.

The fluorescein of the bispecific adapter can bind the anti-fluorescein CAR-T cell with affinity upon exposure thereto, and the targeting ligand of the bispecific adapter can link the bound anti-fluorescein CAR-T cell to a targeted cancer cell upon the targeted ligand of the bispecific adapter binding a receptor on such targeted cancer cell with affinity. In this manner, the conjugates and compositions hereof facilitate enhanced efficacy of CAR-T cell therapy.

In certain embodiments, the receptor on the targeted cancer cell is an overexpressed CAIX. The cancer can be a CAIX-expressing cancer and at least one bispecific adapter of (ii) can comprise a radical of a CAIX ligand.

Also provided are methods for treating CAIX-expressing cancer in a subject. In certain embodiments, the method comprises administering to the subject cancer-treatment effective amounts of (i) anti-fluorescein CAR-T cells or a pharmaceutical composition comprising anti-fluorescein CAR-T cells and a pharmaceutically acceptable carrier or excipient; and (ii) any bispecific adapter or a pharmaceutical composition comprising a bispecific adapter and a pharmaceutically acceptable carrier or excipient. As noted above, the CAR can have a recognition region, and the recognition region is a scFv region of an anti-fluorescein antibody. In certain embodiments, the CAR comprises: a co-stimulation domain, and the co-stimulation domain is CD28, CD137 (4-1BB), CD134 (OX40), or CD278 (ICOS); and/or an activation signaling domain and the activation signaling domain is a T cell CD3ζ chain or an Fc receptor γ.

The methods hereof can further comprise imaging the cancer in the subject. Imaging the cancer can comprise imaging by optical imaging, positron emission tomography (PET), or single photon emission computed tomography (SPECT), for example.

In the methods described herein, the cancer can additionally be imaged prior to administration to the subject of the bispecific adapter, or the pharmaceutically acceptable salts or hydrates thereof, or the engineered cell composition (e.g., a CAR-expressing cytotoxic lymphocyte composition or a CAR-NK cell composition). The cancer additionally, or alternatively, can be imaged during or after administration to assess metastasis, for example, and the efficacy of treatment. For example, imaging can occur by PET imaging, magnetic resonance imaging (MRI), or SPECT/computed tomography (CT) imaging. The imaging method can be any suitable imaging method known in the art.

The cancer can be any cancer. “Cancer” has its plain and ordinary meaning when read in light of the specification and can include, but is not limited to, a group of diseases involving abnormal cell growth with the potential to invade or spread (i.e., metastasize) to other parts of the body. Examples include, but are not limited to, a cancer of the brain, thyroid, lung, pancreas, kidney, stomach, gastrointestinal stroma, endometrium, breast, cervix, ovary, colon, or prostate, leukemias, lymphomas, other blood-related cancers, and head and neck cancer. In certain embodiments, the cancer being treated is a tumor. In certain embodiments, the cancer is malignant. In certain embodiments, the cancer is ovarian cancer, endometrial cancer, breast cancer, glioma such as, optionally, stage 3-4 glioma, or clear cell renal cell carcinoma such as, optionally, stage 3-4 clear cell renal cell carcinoma.

In some aspects of these embodiments, the cancer is a CAIX-expressing cancer.

In some embodiments, the cancer is imaged prior to administration of (i) and (ii) to the subject. Imaging can be done by PET, MRI or SPECT/CT for example.

In certain embodiments, a use of a bispecific adaptor, a pharmaceutically acceptable salt, hydrate, or solvate of the bispecific adaptor, or a composition in the manufacture of a medicament for the treatment of cancer in a subject is provided. The bispecific adaptor can be any conjugate. The medicament can be for use in combination with administration of an engineered cell therapy to the subject such as, for example, CAR T-cell therapy, wherein the CAR T-cells express anti-fluorescein.

Still further, a method for enhancing CAR-T cell activation is provided. The method can comprise providing a bispecific adapter hereof, a pharmaceutical composition comprising same, or a combination thereof (e.g., a therapeutically effective amount of any of the foregoing); and exposing anti-fluorescein CAR-T cells or a pharmaceutical composition comprising anti-fluorescein CAR-T cells and a pharmaceutically acceptable carrier or excipient to the bispecific adaptor(s), pharmaceutical composition, or combination; wherein the CAR-T cell experiences enhanced activation following exposure as compared to a CAR-T cell not exposed to the bispecific adapter. The anti-fluorescein CAR-T cells can be in systemic circulation in a subject when exposed to the bispecific adaptor, pharmaceutical composition, or combination, for example. Alternatively, the anti-fluorescein CAR-T cells can be exposed in vitro to the bispecific adaptor, pharmaceutical composition, or combination (e.g., prior to administration to a subject).

### Kits

Still further provided is a kit. The kit can comprise (i) a bispecific adapter or a pharmaceutical composition comprising the same and a pharmaceutically acceptable carrier or excipient, and (ii) anti-fluorescein CAR-T cells (e.g., anti-FITC CAR-T cells) or a pharmaceutical composition comprising the same and a pharmaceutically acceptable carrier or excipient. In certain embodiments, the bispecific adaptor (or pharmaceutical composition comprising the same) and the CAR-T cells (or pharmaceutical composition comprising same) are stored in separate containers.

### General

Those skilled in the art will recognize that numerous modifications can be made to the specific implementations described above. The implementations should not be limited to the particular embodiments described. Other implementations may be possible.

While the bispecific adapters and pharmaceutical compositions are illustrated and described in detail in the foregoing description, the same is to be considered as illustrative and not restrictive in character, it being understood that only certain embodiments have been shown and described and that all changes and modifications that come within the spirit of the invention are desired to be protected.

It is intended that that the scope of the present bispecific adapters, compositions, and methods are defined by the following claims. However, this disclosure may be practiced otherwise than is specifically explained and illustrated without departing from its spirit or scope. Those skilled in the art will understand that various alternatives to the embodiments described herein can be employed in practicing the claims without departing from the spirit and scope as defined in the following claims.

Any use of section headings is intended to aid reading of the document and is not to be interpreted as limiting. Further, information that is relevant to a section heading may occur within or outside of that particular section.

All publications, patents, patent application publications, journal articles, textbooks, and other publications referred to in this document are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

Various techniques and mechanisms will sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

### Certain Definitions

As used herein, the following terms and phrases shall have the meanings set forth below. Unless defined otherwise, all technical and scientific terms used herein have the same meaning as commonly understood to one of ordinary skill in the art.

The term “about” or “approximately” means within an acceptable range for the particular value as determined by one of ordinary skill in the art, which will depend in part on how the value is measured or determined. e.g., the limitations of the measurement system. For example. “about” can mean a range of up to 20%, preferably up to 10%, more preferably up to 5%, and more preferably still up to 1% of a given value. By way of further example, “about” or “approximately” can mean within 90%, within 95%, 99%, 99.5%, 99.9%, 99.99%, or at least about 99.999% or more of a stated value or of a stated limit of a range. Alternatively, particularly with respect to biological systems or processes, the term can mean within an order of magnitude, preferably within 5-fold, and more preferably within 2-fold, of a value. Unless otherwise stated, the term “about” means within an acceptable error range for the particular value, such as ±1-20%, preferably ±1-10% and more preferably ±1-5%.

Where a range of values is provided, it is understood that each intervening value, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both limits, ranges excluding either or both of those limits are also included.

A phrase referring to “at least one of” a list of items refers to any combination of those items, including single members. As an example, “at least one of a, b, or c” is intended to cover: a, b, c, a-b, a-c, b-c, and a-b-c.

The terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation.

The term “or” is used to refer to a nonexclusive “or” unless otherwise indicated. In addition, it is to be understood that the phraseology or terminology employed herein, and not otherwise defined, is for the purpose of description only and not of limitation.

The terms and expressions employed are used as terms of description and not of limitation. Where certain terms are defined and are otherwise described or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings may be used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

### EXAMPLES

The following examples serve to illustrate the present disclosure. The examples are not intended to limit the scope of the claimed invention in any way.

### Example 1

### General Procedure for the Synthesis of ortho-CAIX-PEG₁-FITC and ortho-(CH₂)₄-FITC Conjugates

The ortho-CAIX-PEG₁-FITC and ortho-(CH₂)₄-FITC conjugates can be synthesized according to Scheme 1.

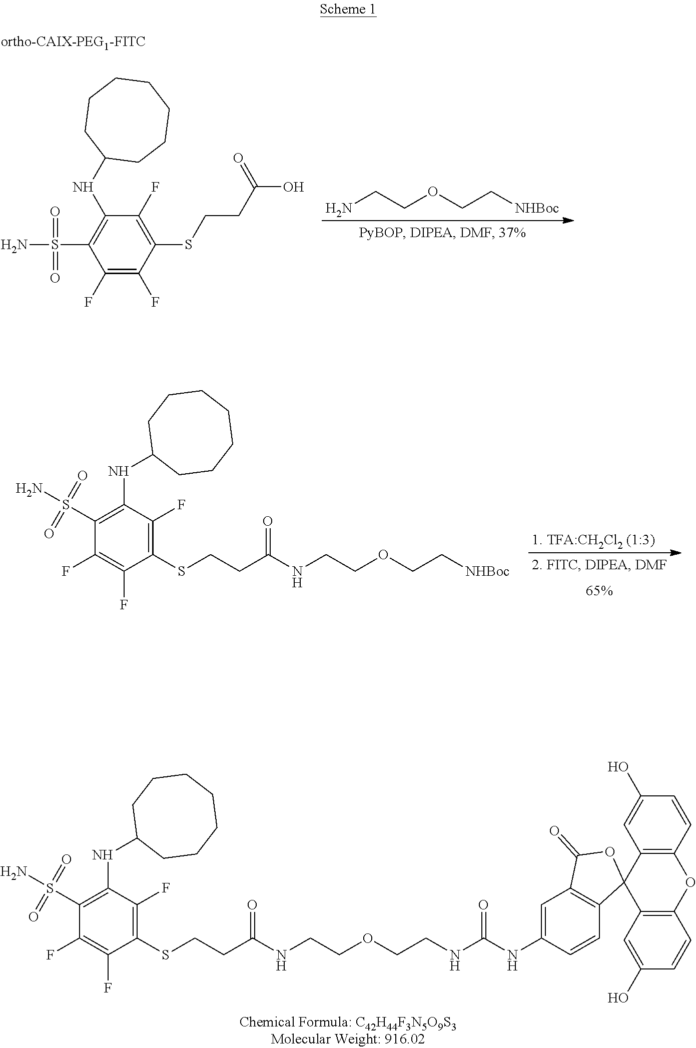 [SMILES: NS(=O)(=O)c1c(F)c(F)c(SCCC(=O)O)c(F)c1NC1CCCCCCC1 ; CC(C)(C)OC(=O)NCCOCCN ; CC(C)(C)OC(=O)NCCOCCNC(=O)CCSc1c(F)c(F)c(S(N)(=O)=O)c(NC2CCCCCCC2)c1F ; NS(=O)(=O)c1c(F)c(F)c(SCCC(=O)NCCOCCNC(=O)Nc2ccc3c(c2)C(=O)OC32c3cc(O)ccc3Oc3ccc(O)cc32)c(F)c1NC1CCCCCCC1]

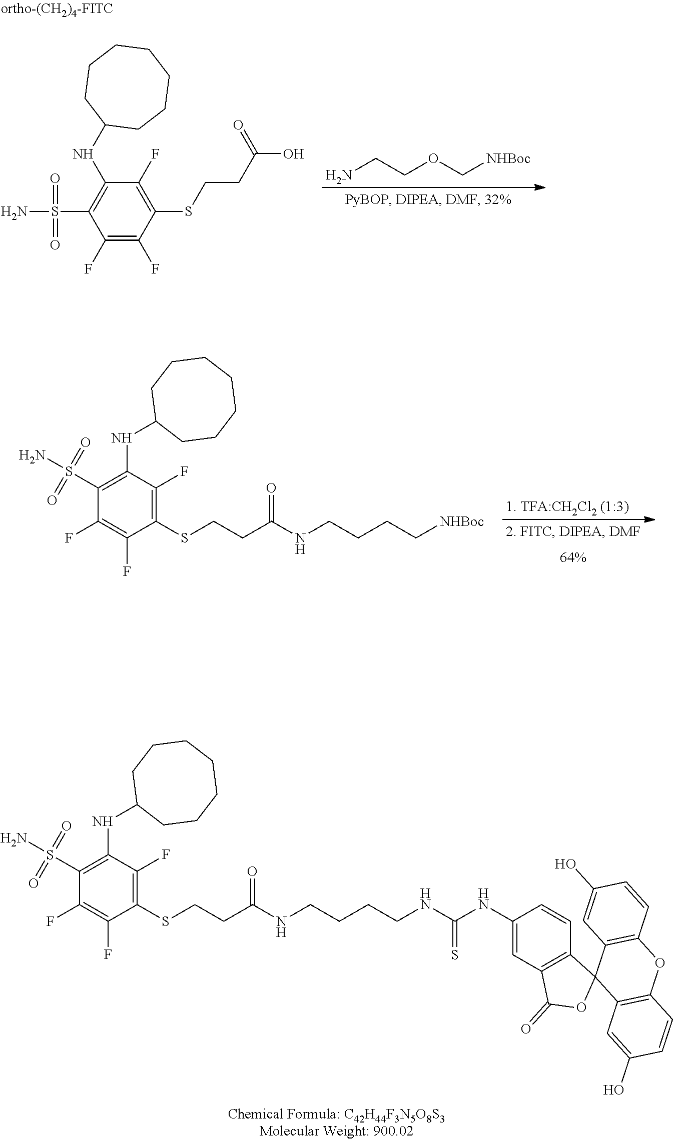 [SMILES: NS(=O)(=O)c1c(F)c(F)c(SCCC(=O)O)c(F)c1NC1CCCCCCC1 ; CC(C)(C)OC(=O)NCOCCN ; CC(C)(C)OC(=O)NCCCCNC(=O)CCSc1c(F)c(F)c(S(N)(=O)=O)c(NC2CCCCCCC2)c1F ; NS(=O)(=O)c1c(F)c(F)c(SCCC(=O)NCCCCNC(=S)Nc2ccc3c(c2)C(=O)OC32c3cc(O)ccc3Oc3ccc(O)cc32)c(F)c1NC1CCCCCCC1]

PyBOP (1.2 eq)+DIPEA (2.0 eq) were added to a stirred solution of acid compound (1.0 eq) in dimethylformamide (DMF). After 10 minutes of stirring, BocNH-PEG1-NH₂ or BocNH(CH₂)₄NH₂ (1.2 eq) were added to the above reaction mixture, and stirring was continued for an additional 2 hours. After the complete conversion of starting materials (measured by liquid chromatography-mass spectrometry (LC-MS)), the reaction mixture was diluted with water, then extracted into dichloromethane (DCM) (2×20 mL), the combined organic extracts were dried over anhydrous sodium sulfate, filtered and the filtrate was evaporated under reduced pressure, then crude residue was obtained and purified by combiflash using (A=Ammonium acetate 10 mM, P^{H}=7.5, B=Acetonitrile) for 40 minutes using 5-95 method. The desired fractions obtained were kept on lyophilizer for 48 hours to yield the coupled product as white solids.

To the lyophilized solids (0.0133 mmol) in DCM (3 mL) was added trifluoroacetic acid (TFA) (1 mL) and stirring was continued there for 30 minutes. Thereafter, the reaction mixture was evaporated under reduced pressure to provide the free amine as a brown color gummy solid. The obtained amine was used in the next step without purification. To a stirred solution of amine (1 eq) in DMF (500 μL) N,N,-Diisopropylethylamine (DIPEA) (10 eq) followed by Fluorescein isothiocyanate isomer 1 (1 eq) were added and stirring was continued at room temperature an additional 2 hours. The reaction mixture was diluted with water and purified by ultra-high performance liquid chromatography (U-HPLC) using (A=Ammonium acetate 10 mM, P^{H}=7.5, B=Acetonitrile) for 60 minutes using 5-35 method and obtained desired fractions were quickly kept on lyophilizer for 48 hours to provide the desired ortho-CAIX-FITC compounds as yellow solids.

### Example 2

### Binding Affinities of Bispecific Adapters Targeting Carbonic Anhydrase IX

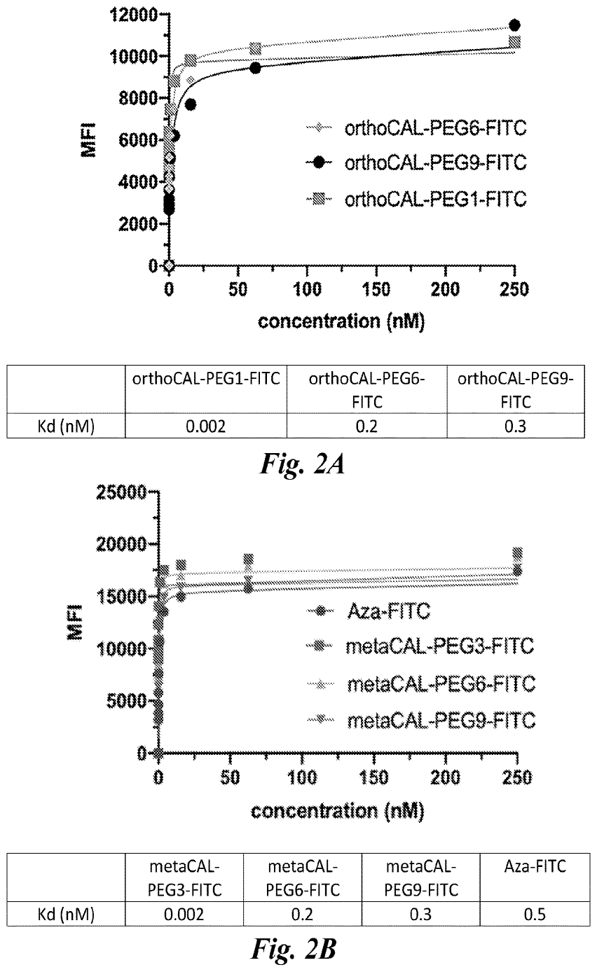

MDA-carbonic anhydrase IX (CAIX) cells (0.2 million) were incubated with two-fold serially diluted bispecific adapters starting with 250 nM in phosphate-buffered saline (PBS) containing 2% fetal bovine serum (FBS) for one hour at room temperature. The cells were washed once to remove unbound adapters. The fluorescent intensity of adapters bound to the cells was analyzed by flow cytometry. The results are shown in FIGS. 2A-2B.

For ortho-CAL, binding affinity to CAIX+ cells decreased with increasing linker length. For meta-CAL, binding affinity to CAIX+ cells slightly decreased with increasing linker length, but all meta-CAL bispecific adapters had higher binding.

### Example 3

### Total Binding and Surface Exposure of CAIX Bispecific Adapters

HT29 cells or MDA-CAIX cells were incubated with 1 μM bispecific adapters with different ligands to CAIX and different polyethylene glycol (PEG) linkers in complete RPMI medium (RPMI+10% FBS) for one hour at room temperature. Free compounds were washed away, and fluorescent intensity of fluorescein isothiocyanate (FITC) was analyzed by flow cytometry.

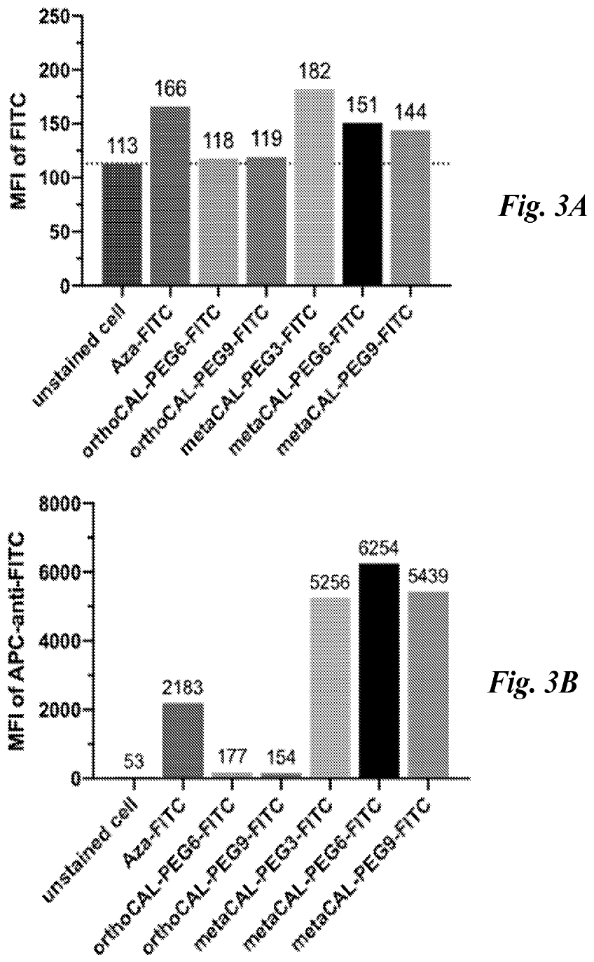

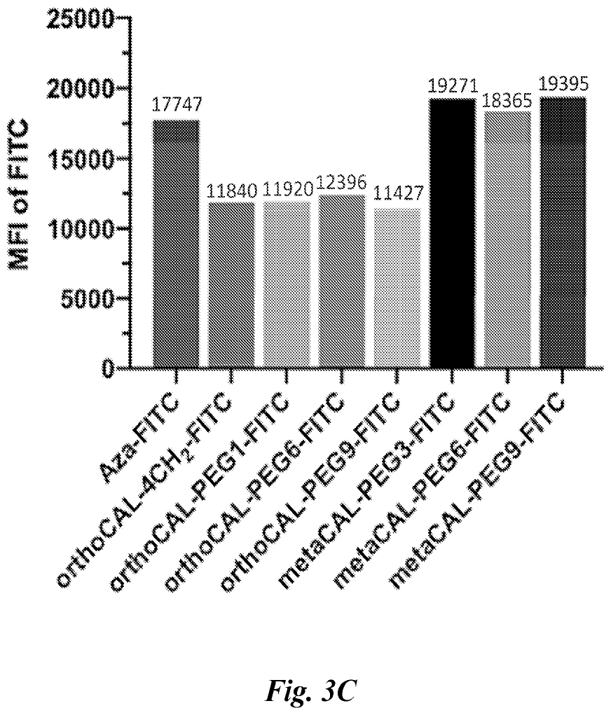

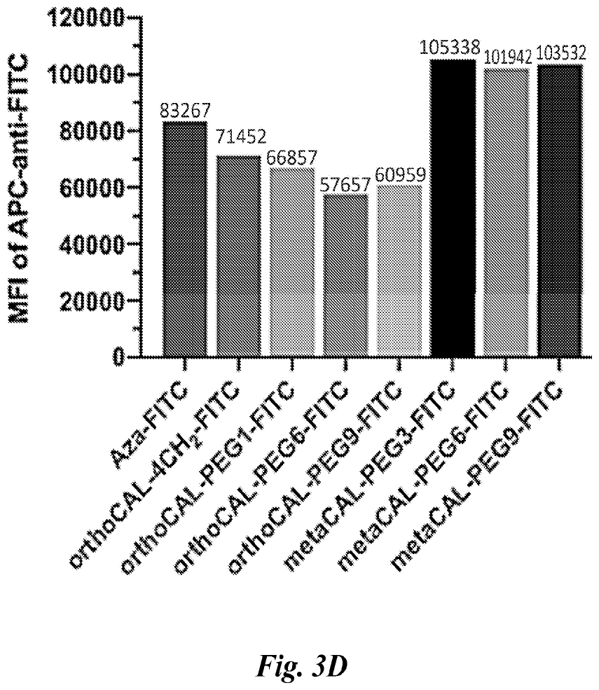

To analyze the surface exposure of the FITC moiety, the stained cells were incubated with allophycocyanin (APC)-anti-FITC antibody on ice for 30 minutes. Then the cells were washed, and the fluorescent intensity of APC was analyzed by flow cytometer. The results are shown in FIGS. 3A-3D. As shown in FIG. 3A, meta-CAL-PEG3 and Aza-FITC had better total binding to HT29 cells than the other adapters. As shown in FIG. 3B, all three meta-CAL-FITC adapters had better surface binding and FITC exposure in HT29 cells. As shown in FIG. 3C, meta-CAL-FITC with different linkers and Aza-FITC had better total binding to MDA-CAIX cells. As shown in FIG. 3D, all the meta-CAL-FITC adapters had better surface binding and FITC exposure that the other tested adapters in MDA-CAIX cells.

### Example 4

### CAIX Targeting Bispecific Adapters Mediated Anti-FITC CAR-T Cell Cytotoxicity and IFNγ Release on MDA-CAIX Cells

Anti-FITC CAR-T cells were incubated with an equal number of MDA-CAIX cells in the presence of bispecific adapters at different concentrations. The number of MDA-CAIX cells was determined at the end of the co-culture, and the percentage of cell lysis was calculated using the following formula: [(number of untreated cells−number of treated cells)/number of untreated cells]*100%.

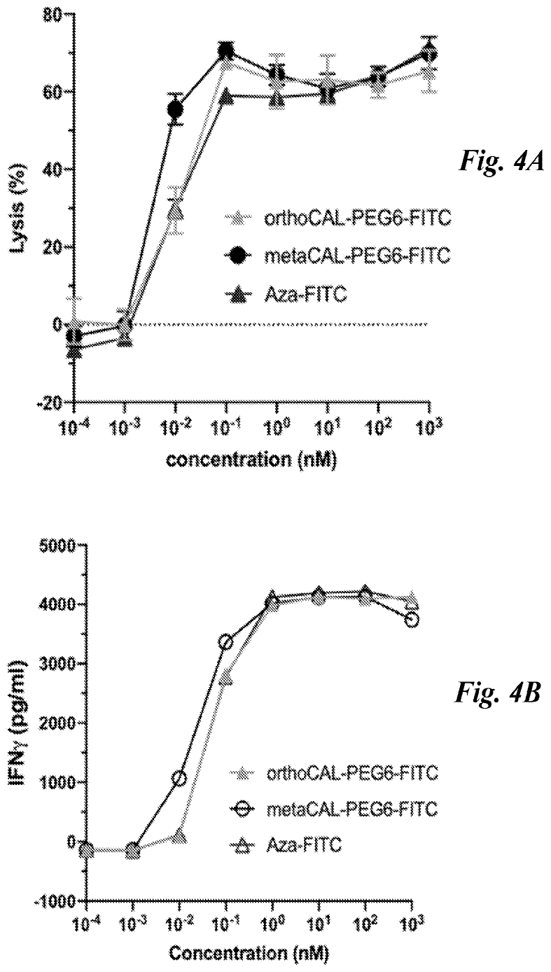

Secretion of interferon gamma (IFNγ) from anti-FITC CAR-T cells was analyzed by enzyme-linked immunosorbent assay (ELISA) using the supernatants of the co-cultured medium at the end of the co-culture. The results are shown in FIGS. 4A-4B.

As shown in FIG. 4A, the efficacy of meta-CAL-PEG₆-FITC is higher than ortho-CAL-PEG₆-FITC and Aza-FITC between 0.001 nM and 10 nM. As shown in FIG. 4B, meta-CAL-PEG₆-FITC mediated a higher level of IFNγ release from anti-FITC CAR-T cells than ortho-CAL-PEG₆-FITC and Aza-FITC when the adapter concentration is between 0.001 nM and 1 nM.

### Example 5

Effect of Linker Length on Meta-CAL-FITC Bispecific Adapter-Mediated Anti-FITC CAR-T Cell Cytotoxicity and IFN, Release when Co-Cultured with MDA-CAIX Cells

Anti-FITC CAR-T cells were incubated with an equal number of MDA-CAIX cells in the presence of the bispecific adapters at different concentrations. The number of MDA-CAIX cells was determined at the end of the co-culture, and the percentage of cell lysis was calculated using the following formula: [(number of untreated cells−number of treated cells)/number of untreated cells]*100%.

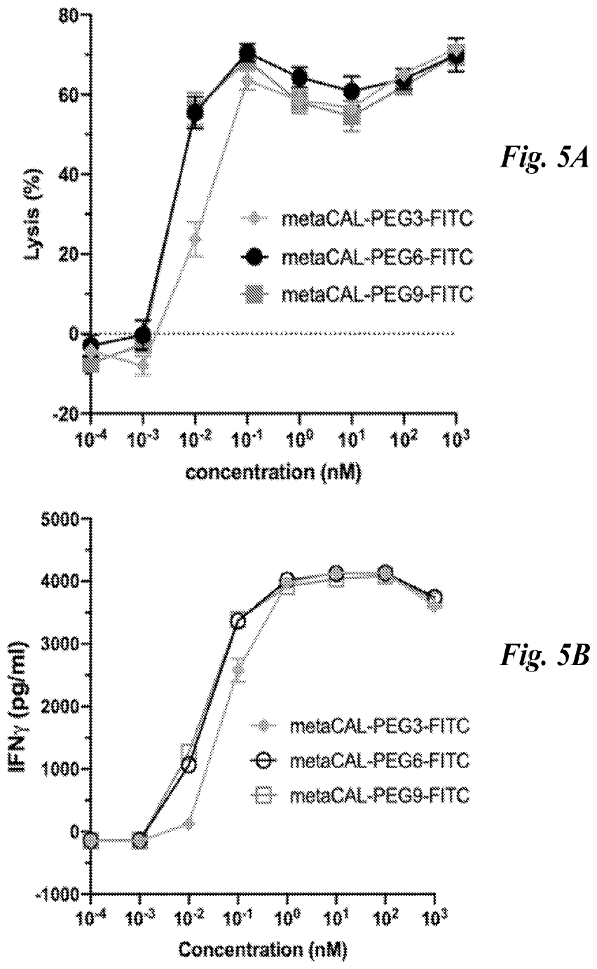

Secretion of IFNγ from anti-FITC CAR T cells was analyzed by ELISA using the supernatants of the co-cultured medium at the end of the co-culture. The results are shown in FIGS. 5A-5B. As shown in FIG. 5A, meta-CAL-PEG₆-FITC and meta-CAL-PEG₉-FITC had higher efficacy than meta-CAL-PEG3-FITC at low concentrations (between 0.001 nM and 0.1 nM). As shown in FIG. 5B, meta-CAL-PEG₆-FITC and meta-CAL-PEG₉-FITC mediated higher levels of IFNγ release from anti-FITC CAR-T cells than meta-CAL-PEG3-FITC at low concentrations (between 0.001 nM and 1 nM).

### Example 6

Effect of Linker Length on Ortho-CAL-FITC Bispecific Adapter-Mediated Anti-FITC CAR-T Cell Cytotoxicity and IFNγ, Release when Co-Cultured with MDA-CAIX Cells

Anti-FITC CAR T cells were incubated with an equal number of MDA-CAIX cells in the presence of the bispecific adapters at different concentrations. The number of MDA-CAIX cells were determined at the end of the co-culture and the percentage of cell lysis was calculated using the following formula: [(number of untreated cells−number of treated cells)/number of untreated cells]*100%.

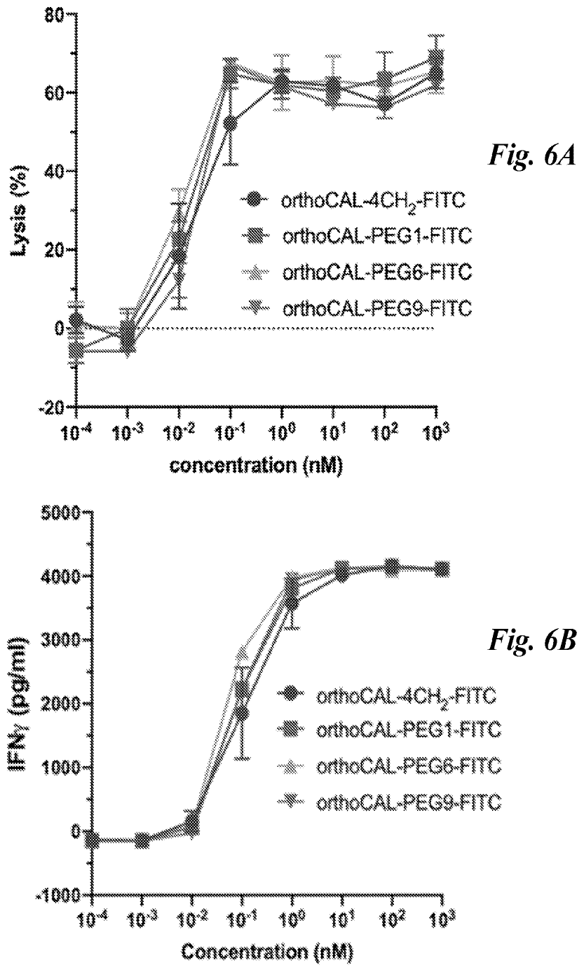

Secretion of IFNγ from anti-FITC CAR T cells was analyzed by ELISA using the supernatants of the co-cultured medium at the end of the co-culture study. The results are shown in FIGS. 6A-6B.

As shown in FIG. 6A, the ortho-CAL-FITC bispecific adapters had similar efficacies, although longer PEG spacers were slightly better than the (CH₂)₄ spacer. As shown in FIG. 6B, the ortho-CAL-FITC bispecific adapters showed similar efficacy, although longer PEG spacers were slightly better than the (CH₂)₄ spacer.

### Example 7

### In Vivo Efficacy of CAIX Targeting Bispecific Adapters on HT29 Tumor Model

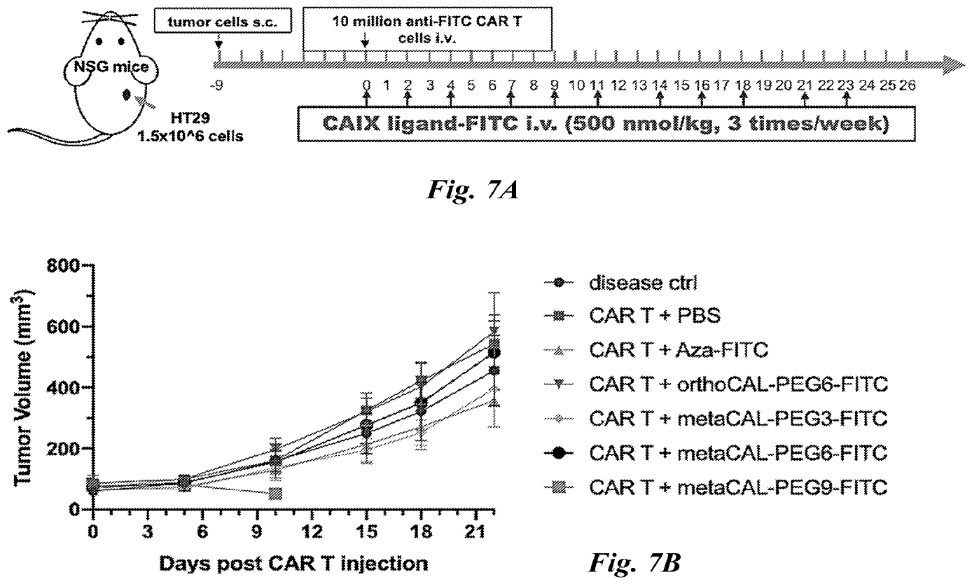

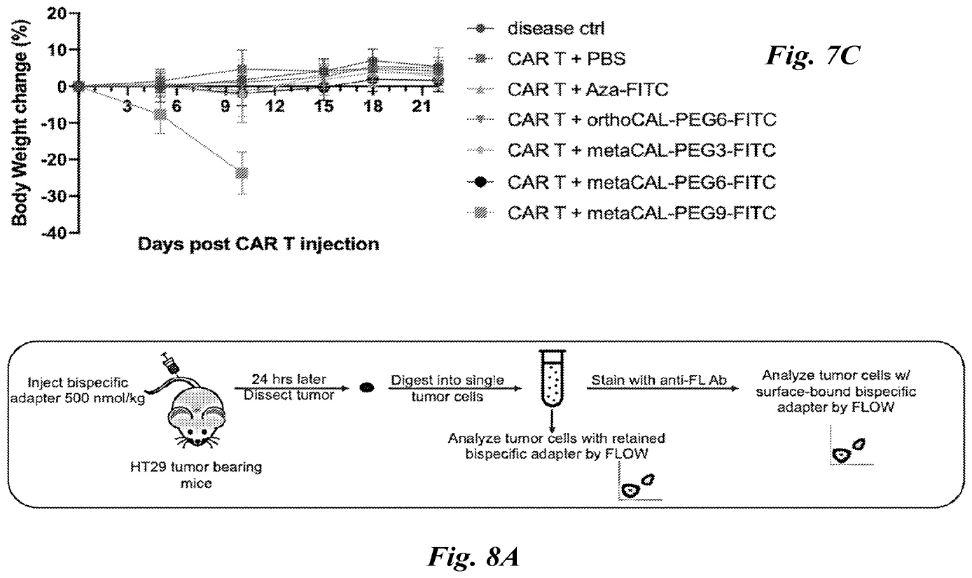

HT29 cells (1.5 million) were implanted into each NOD scid gamma (NSG) mouse by subcutaneous injection. When the tumor volumes reached about 100 mm³, the treatment groups were injected with 10 million anti-FITC CAR-T cells and bispecific adapters as shown in FIG. 7A. The tumor volume and body weight were monitored regularly. Tumor volume was calculated using the formular: (length*width²)/2. The results are shown in FIGS. 7B-7C.

As shown in FIG. 7B, Aza-FITC and meta-CAL-PEG₃-FITC slightly inhibited the growth of HT29 tumor, whereas meta-CAL-PEG₉-FITC significantly inhibited the growth of HT29 tumor. As shown in FIG. 7C, only mice treated with meta-CAL-PEG₉-FITC lost body weight. Loss of body weight could be due to cytokine release from expanded CAR-T cells, which is an indication of better functionality of meta-CAL-PEG₉-FITC. Toxicity can be minimized by optimizing dosing of the adapter.

### Example 8

### Retention and Surface Exposure of CAIX Bispecific Adapters in HT29 Tumor Cells

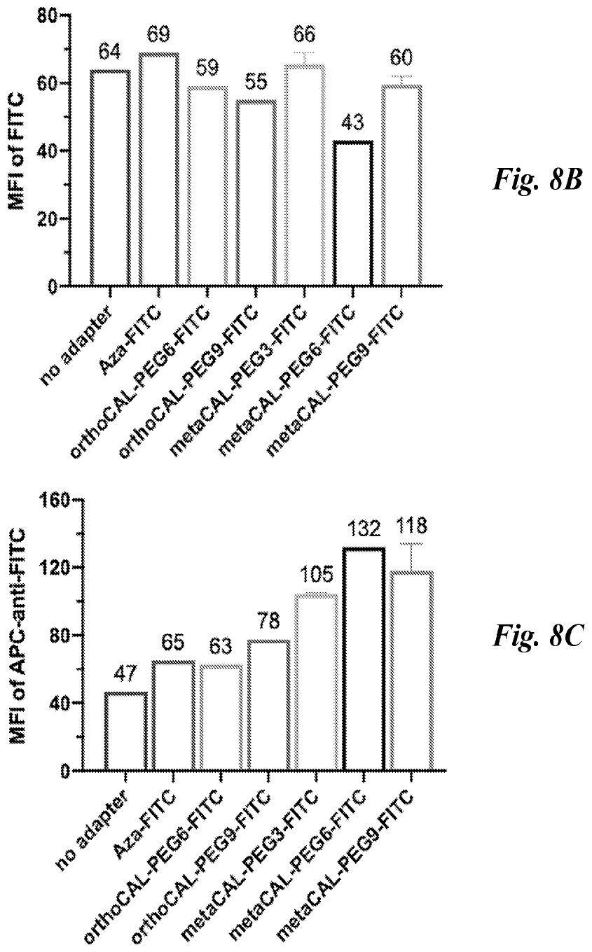

HT29 tumor bearing mice were injected with CAIX bispecific adapters at 500 nmol/kg. After injection (24 hours later), the tumors were dissected and digested into single cells. The total retention of the bispecific adapters was determined by the FITC fluorescence intensity analyzed by flow cytometry. To analyze the FITC exposure of the bispecific adapters on the tumor cell surface, the digested tumor cells were stained with APC-anti-FITC antibody on ice for 30 minutes. After washing away unbound antibody, the fluorescent intensity of APC was analyzed by flow cytometry. The results are shown in FIGS. 8B-8C.

As shown in FIG. 8B, all compounds had very low retention in HT29 tumor; this could be due to low expression of CAIX in HT29 tumor cells. As shown in FIG. 8C, meta-CAL-FITC with different linkers had better surface retention and FITC exposure than the other compounds. Meta-CAL-PEG₆-FITC and meta-CAL-PEG₉-FITC had better FITC exposure than meta-CAL-PEG₃-FITC in vivo.

### Example 9

T Cell Counts in the Blood and HT29 Tumors from Mice at End of Treatments

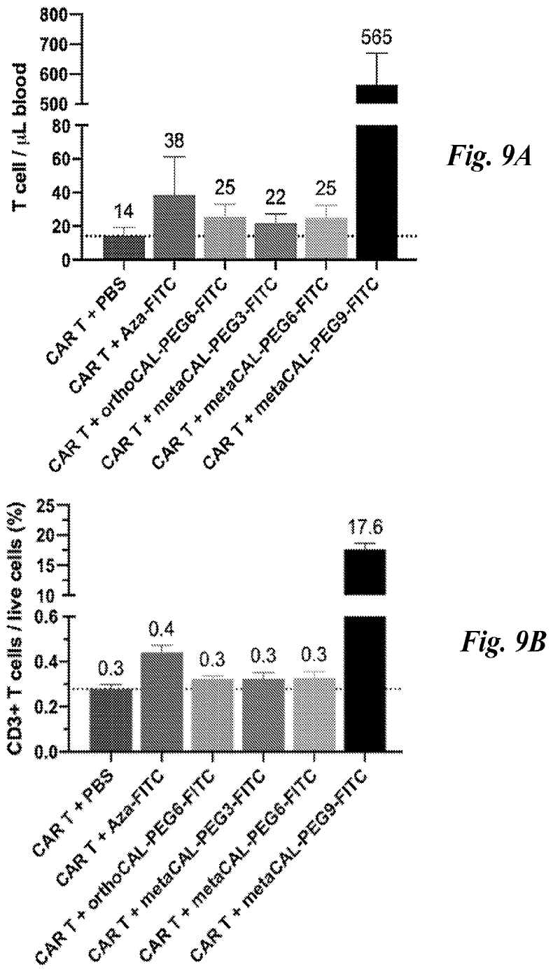

At the end of the in vivo study on HT29 solid tumor model described in FIGS. 7A-7C, blood was collected from the mice, and the red blood cells in the blood were lysed by using red blood cell lysis buffer (BioLegend, San Diego, CA). The remaining cells were stained with anti-human CD3 antibody on ice for 30 minutes. The number of T cells was analyzed by flow cytometry. To analyze tumor-infiltrated T cells, tumors were dissected and digested to single cells. The digested cells were stained with Zombie Violet cell viability staining dye (BioLegend. San Diego, CA) and anti-human CD3 antibody. The percentage of CD3 T cells in total live cells was analyzed by flow cytometry. The results are shown in FIGS. 9A-9B.

As shown in FIG. 9A, blood from mice treated with meta-CAL-PEG₉-FITC had more T cells than the blood from other treatment groups. As shown in FIG. 9B, there were more tumor-infiltrated T cells in the tumors from mice treated with meta-CAL-PEG₉-FITC.

### Example 10

### In Vivo Efficacy of CAIX Targeting Bispecific Adapters on MDA-CAIX Tumor Model

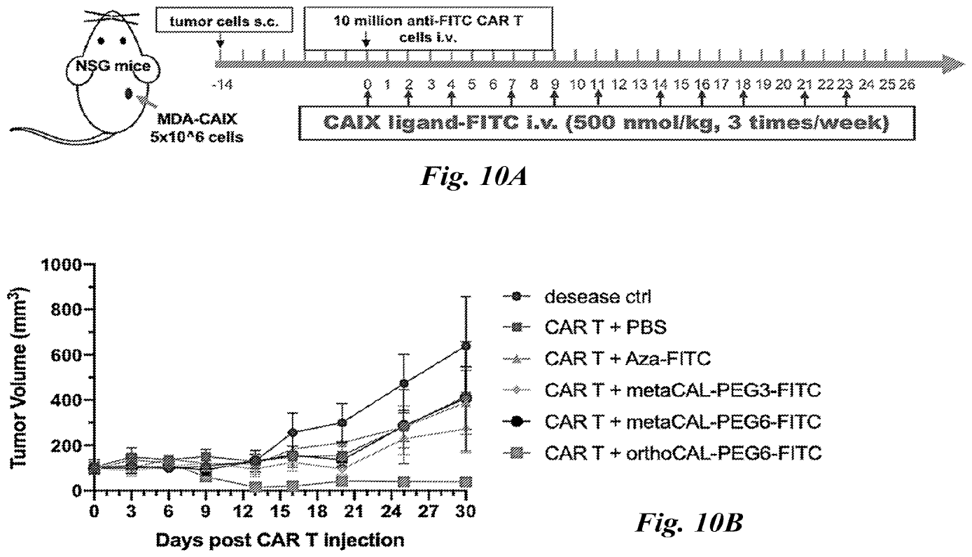

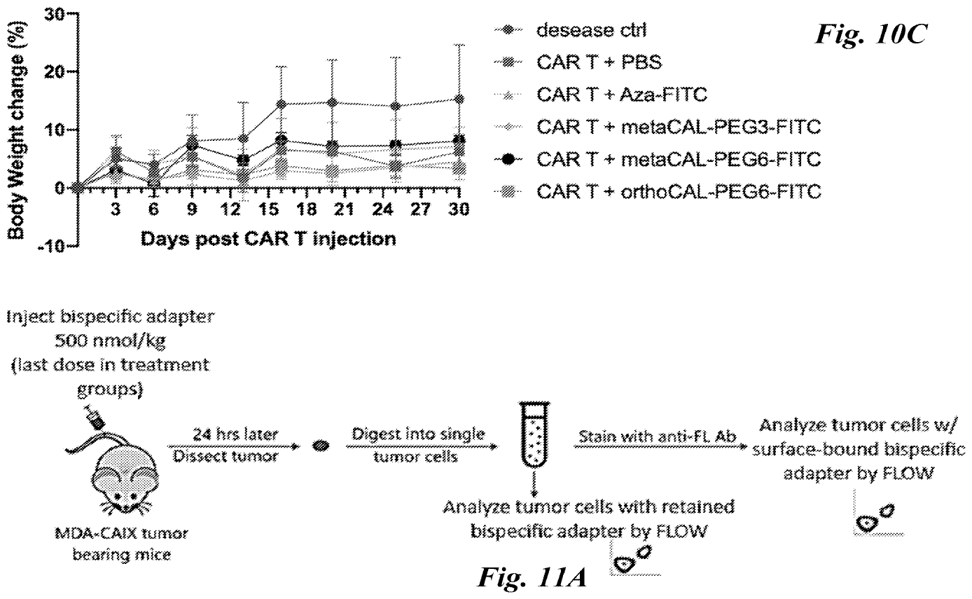

MDA-CAIX cells (5 million) were implanted into each NSG mouse by subcutaneous injection. When the tumor volumes reached about 100 mm³, the treatment groups were injected with 10 million anti-FITC CAR T-cells and indicated bispecific adapters as shown in FIG. 10A. The tumor volume and body weight were monitored regularly. Tumor volume was calculated using the formular: (length*width²)/2. The results are shown in FIGS. 10B-10C.

As shown in FIG. 10B, meta-CAL-PEG3-FITC slightly inhibited the growth of MDA-CAIX tumors, whereas ortho-CAL-PEG₆-FITC significantly inhibited the growth of MDA-CAIX tumors. As shown in FIG. 10C, none of the bispecific adapters showed toxicity to the mice during treatment.

### Example 11

### Retention and Surface Exposure of CAIX Bispecific Adapters in MDA-CAIX Tumor Cells

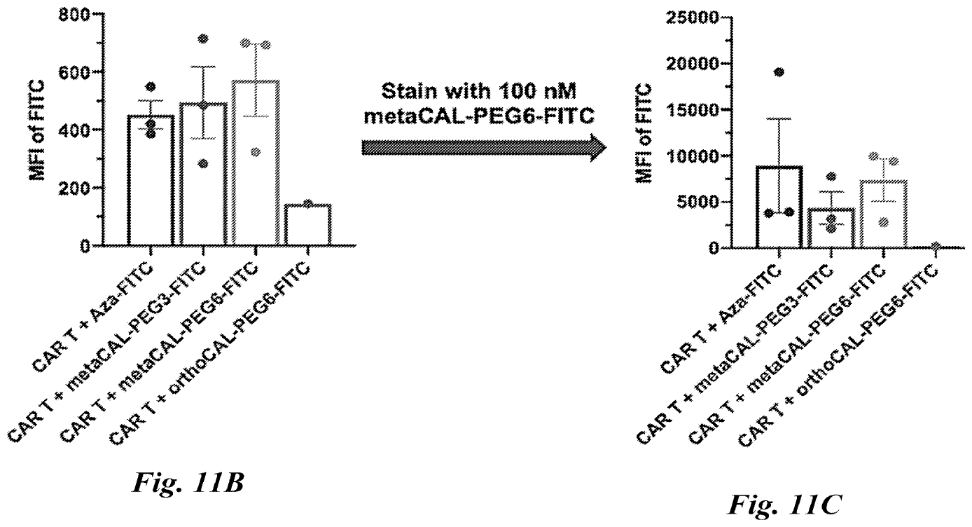

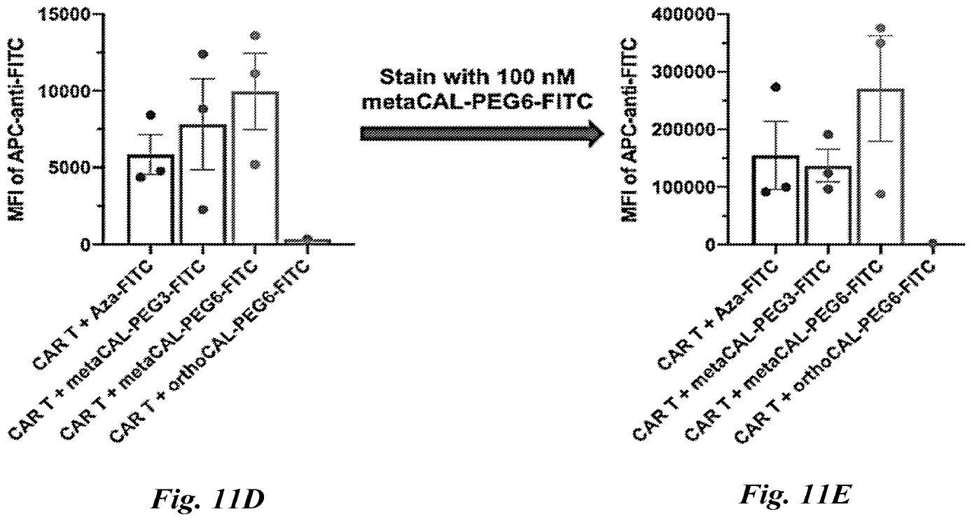

MDA-CAIX tumors from mice in different treatment groups were dissected and digested into single cells 24 hours after the last injections with CAIX bispecific adapters at 500 nmol/kg. The total retention of the bispecific adapters was determined by the FITC fluorescence intensity analyzed by flow cytometry. To estimate the CAIX protein levels in the tumor cells, the cells were stained with 100 nM meta-CAL-PEG₆-FITC for 1 hour at room temperature and analyzed by flow cytometry. The FITC exposure of the bispecific adapters on the tumor cell surface was analyzed by flow cytometry after staining with APC-anti-FITC antibody on ice for 30 minutes. Surface exposure of FITC was also analyzed in the cells stained with 100 nM meta-CAL-PEG₆-FITC. The results are shown in FIGS. 11B-11E.

As shown in FIG. 11B, tumors treated with Aza-FITC and meta-CAL-FITC with PEG₃ or PEG₆ linkers had similar total FITC retention in the cells. As shown in FIG. 11C, tumor cells treated with Aza-FITC had slightly higher CAIX levels than tumors treated with meta-CAL-FITC with PEG₃ or PEG₆. As shown in FIG. 11D, meta-CAL-PEG₆-FITC had slightly better surface retention and FITC exposure than the other compounds. As shown in FIG. 11E, tumor cells from mice treated with ortho-CAL-PEG₆-FITC had almost no staining of anti-FITC antibody, indicating that the tumor cells expressing CAIX had been killed by CAR-T cells.

### Example 12

### General Procedure for the Synthesis of Acetazolamide (Aza)-PEG-FITC Conjugates

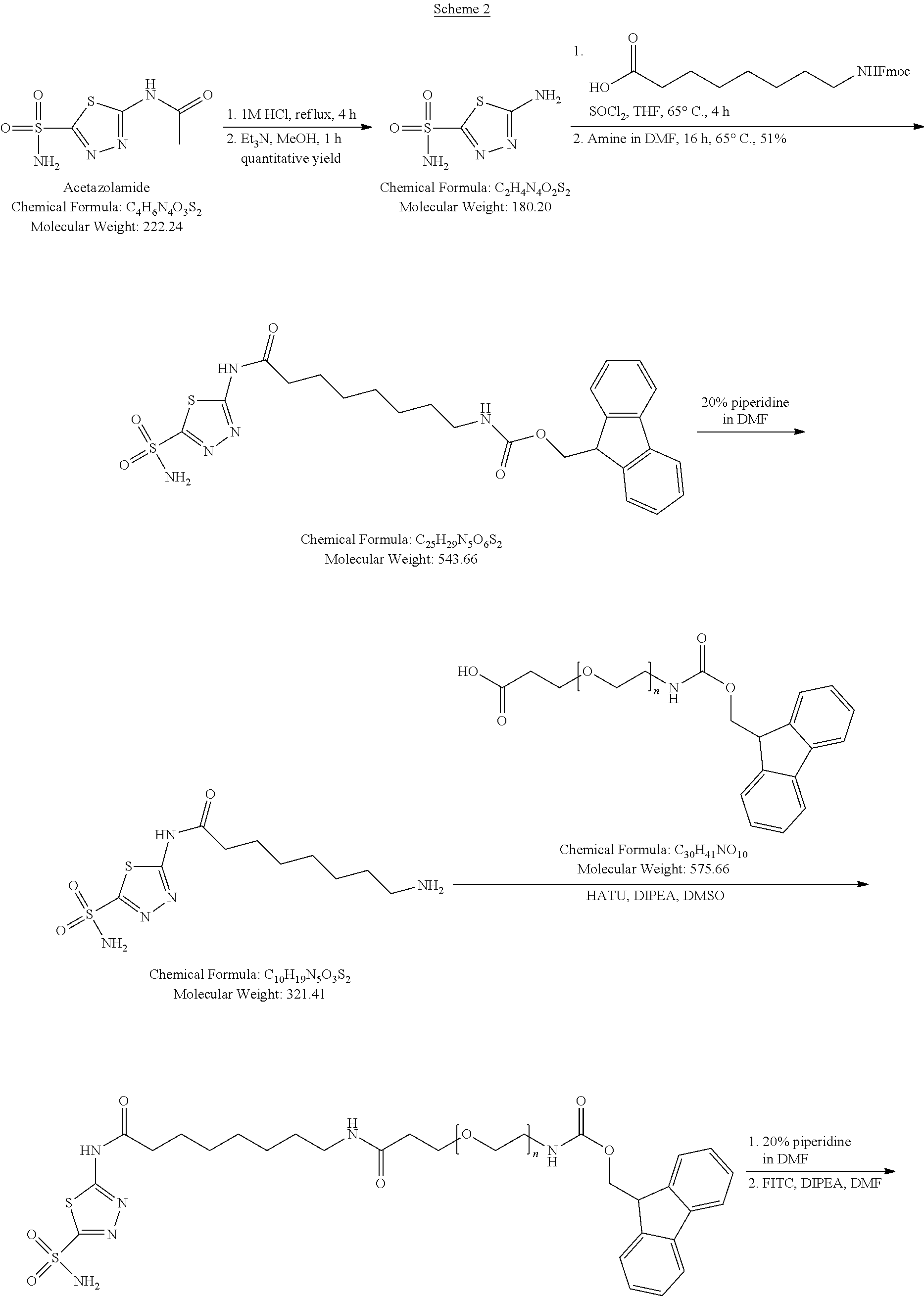

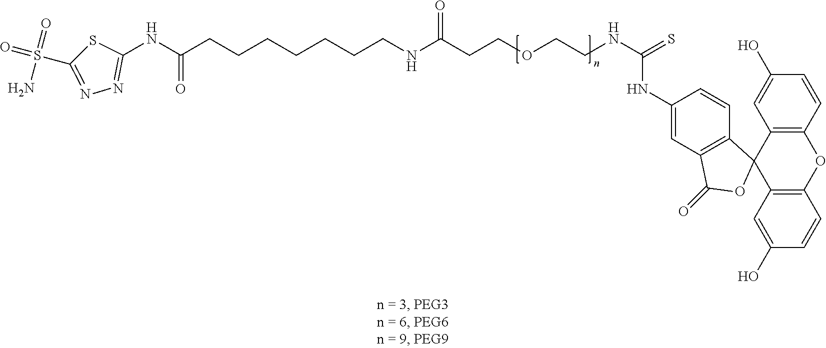

### Example 13

### Effects of Linker Length on the Binding Affinity of Aza-FITC Bispecific Adapters

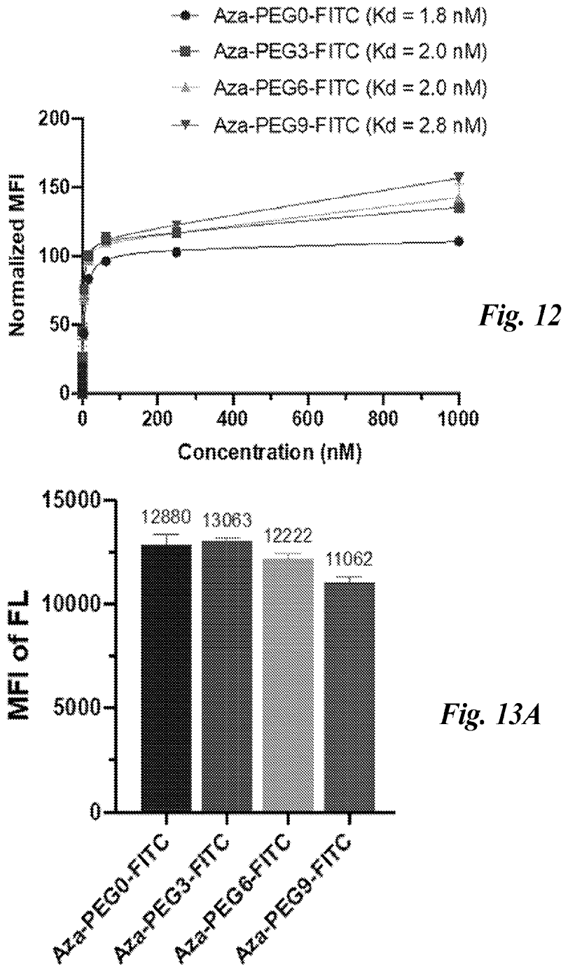

MDA-CAIX cells (0.2 million) were incubated with 4-fold serial diluted bispecific adapters starting with 1000 nM in PBS containing 2% FBS for 1 hour at room temperature. The cells were washed once to remove unbound adapters. The fluorescent intensity of adapters bound to the cells were analyzed by flow cytometry. The results are shown in FIG. 12, which is a graph of concentration (nM) vs. normalized MFI that shows that PEG spacers do not have significant effects on the binding affinity of acetazolamide (Aza)-FITC bispecific adapters.

### Example 14

### Effects of Linker Length on Total Binding and Surface Exposure of Aza-FITC Bispecific Adapters

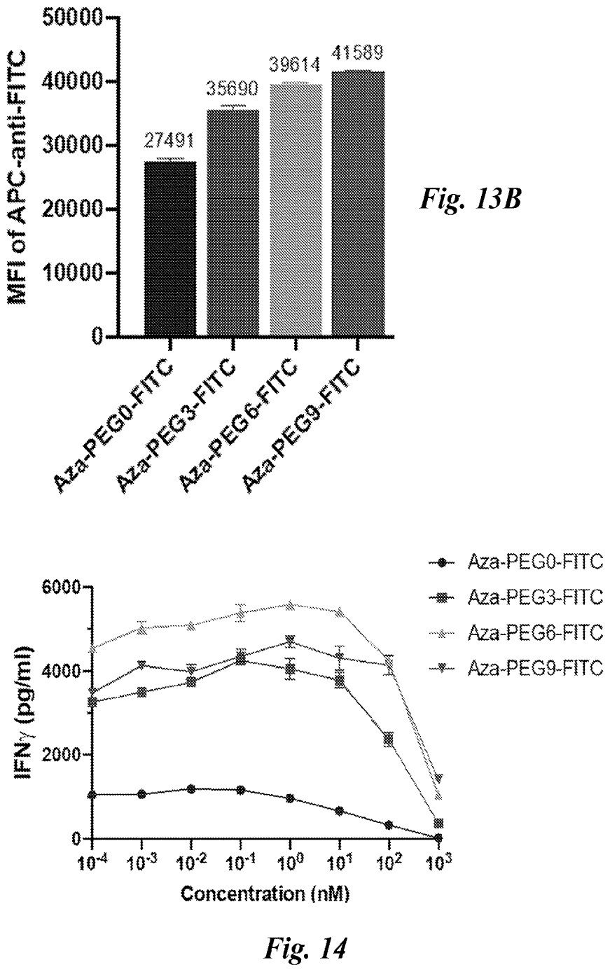

MDA-CAIX cells were incubated with 500 nM Aza-FITC bispecific adapters with different PEG spacers in complete RPMI medium (RPMI+10% FBS) for 1 hour at room temperature. Free compounds were washed away and fluorescent intensity of FITC were analyzed by flow cytometry. To analyze the surface exposure of FITC moiety, the stained cells were incubated with APC-anti-FITC antibody on ice for 30 minutes. Then the cells were washed, and the fluorescent intensity of APC were analyzed by flow cytometer. The results are shown in FIGS. 13A-13B. FIG. 13A is a graph of bispecific adapter vs. MFI of FL, which shows the total binding of Aza-FITC bispecific adapters to MDA-CAIX cells. Linker length did not significantly affect the total binding. FIG. 13B is a graph of bispecific adapter vs. MFI of APC-anti-FITC, which shows the surface exposure of FITC moieties in Aza-FITC bispecific adapters with different PEG linkers after binding to MDA-CAIX cells. Increased PEG spacer length enhanced surface exposure of FITC after binding to MDA-CAIX cells.

### Example 15

Effects of Linker Length on Aza-FITC Bispecific Adapter Mediated Anti-FITC CAR-T Cell IFNγ Release when Co-Cultured with HT 29 Cells

Anti-FITC CAR-T cells were incubated with HT29 cells at 1:5 ratio in the presence of the bispecific adapters at different concentrations for 40 hours. Secretion of IFNγ from anti-FITC CAR-T cells was analyzed by ELISA using the supernatants of the co-cultured medium at the end of the co-culture study. The results are shown in FIG. 14, which is a graph of concentration (nM) vs. IFNγ (pg/ml), which shows the effect of linker length on Aza-FITC bispecific adapter mediated anti-FITC CAR-T cell IFNγ release when co-cultured with HT 29 cells.

Aza-PEG3-FITC and Aza-PEG9-FITC mediated higher level of IFNγ released from anti-FITC CAR-T cells than Aza-PEG₀-FITC. Aza-PEG₆-FITC mediated the highest level of IFNγ released from anti-FITC CAR-T cells.

### Example 16

CAIX-Targeting Bispecific Adapters with Optimal Linkers Mediated Anti-4TC CAR-T Cell IFNγ Release

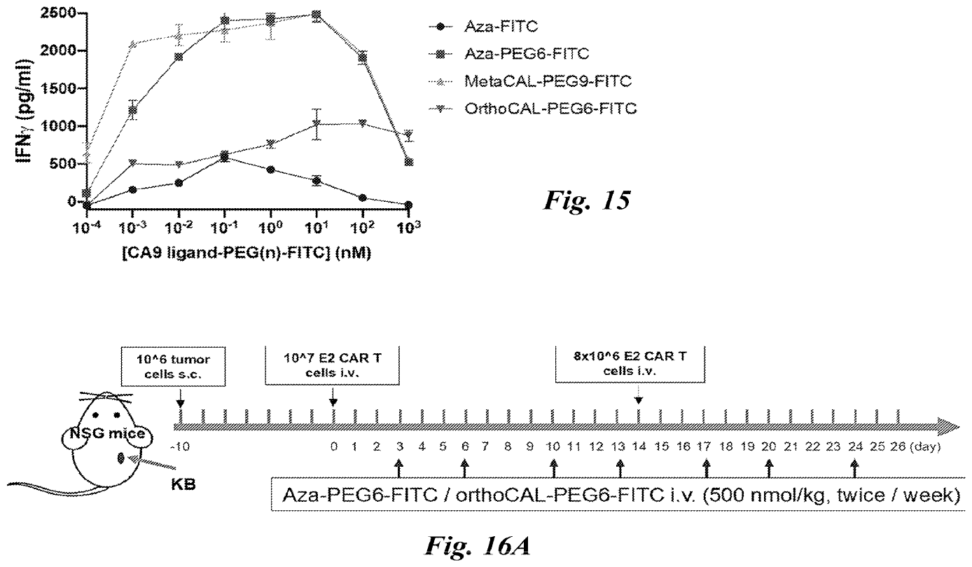

Anti-FITC CAR T cells were incubated with HT29 cells at 1:5 ratio in the presence of the bispecific adapters at different concentrations for 40 hours. Secretion of IFNγ from anti-FITC CAR T cells was analyzed by ELISA using the supernatants of the co-cultured medium at the end of the co-culture study. The results are shown in FIG. 15, which is a graph of concentration of CA9 ligand-PEG(n)-FITC (nM) vs. IFNγ (pg/ml), which shows CAIX-targeting bispecific adapters with optimal linkers mediated anti-FITC CAR-T cell IFNγ release. MetaCAL-PEG9-FITC mediated the highest level of IFNγ released from anti-FITC CAR-T cells at low concentrations (<0.1 nM). Aza-PEG₆-FITC mediated similar levels of IFNγ release from anti-FITC CAR T-cells as metaCAL-PEG₉-FITC at concentrations higher than 0.1 nM. OrthoCAL-PEG₆-FITC mediated lower levels of IFNγ released from anti-FITC CAR-T cells.

### Example 17

In Vivo Efficacy of Aza-PEG₆-FITC and orthoCAL-PEG₆-FITC

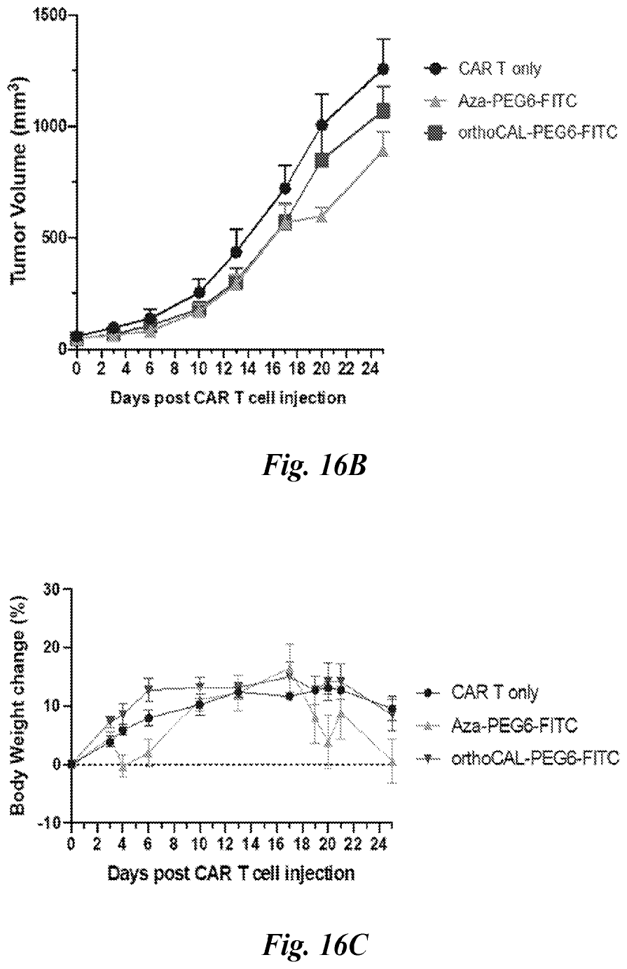

KB cells (1 million) were implanted into each NSG mouse by subcutaneous injection. When the tumor volumes reached about 50 mm³, the treatment groups were injected with 10 million anti-FITC CAR-T cells and indicated bispecific adapters as shown in FIG. 16A. The tumor volume and body weight were monitored regularly. Tumor volume was calculated using the formula: (length*width²)/2. The results are shown in FIGS. 16B-16C.

FIG. 16B is a graph of days post-CAR-T cell injection vs. tumor volume (mm), which shows tumor growth curves of different treatment groups. Aza-PEG₆-FITC and orthoCAL-PEG₆-FITC both slightly inhibited the growth of KB tumors. The efficacy of Aza-PEG₆-FITC is slightly better than that of orthoCAL-PEG₆-FITC. FIG. 16C is a graph of days post-CAR-T cell injection vs. body weight change (%), which shows body weight changes of mice in different treatment groups. Both Aza-PEG₆-FITC and orthoCAL-PEG₆-FITC did not induce significant body weight loss.

### Example 18

### Synthesis of Aza-FITC

Synthesis of 5-amino-2-sulfamoyl-1,3,4-thiadiazole monohydrochloride B: Hydrochloric acid (1M aq.) (70 mL, 70.00 mmol, 5.2 equiv) was added to acetazolamide A (3 g, 13.34 mmol, 1.0 equiv) and the mixture was stirred for 3 hours at reflux (Scheme 3). Thereafter, water was evaporated by rotavapor. The crude material (dissolved in small amount of methanol with the help of TEA for solid loading) was purified by column chromatography (CHCl₃/MeOH: 100/0 to 70/30) to yield product B (2.768 g, 96%).

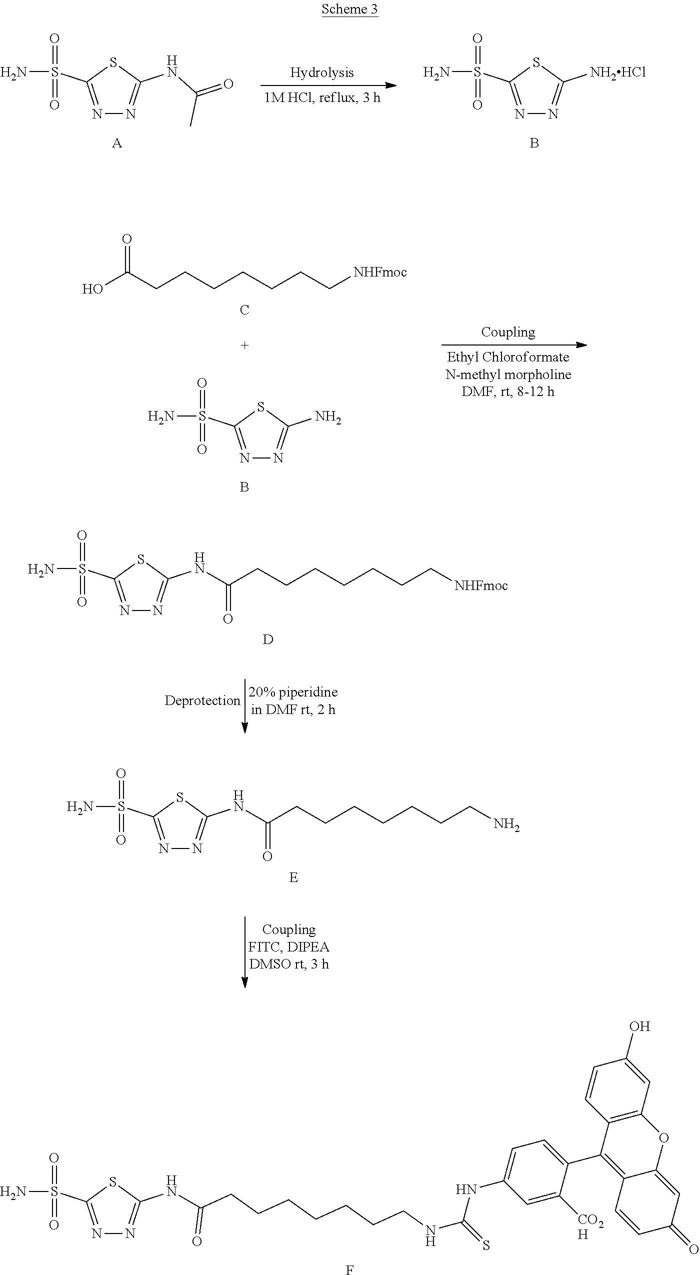

Synthesis of (9H-fluoren-9-yl)methyl (8-oxo-8-((5-sulfamoyl-1,3,4-thiadiazol-2-yl)amino)octyl)carbamate D: N-methyl morpholine (0.063 mL, 1.1 equiv) was added at room temperature and under argon atmosphere to a stirred solution of the N-Fmoc-8-aminooctanoic acid C (0.200 g, 1 equiv) and ethylchloroformate (0.055 mL, 1.1 equiv) in freshly dried DMF (1.5 mL), and thereafter stirred for 30 minutes (Scheme 3). 5-amino-2-sulfamoyl-1,3,4-thiadiazole B (0.568, 1.5 equiv) was then added at room temperature and stirring was continued overnight under argon. The reaction was monitored by liquid chromatography-mass spectrometry (LCMS) and purified by preparative high performance liquid chromatography (HPLC) (mobile phase: A=20 mM ammonium acetate pH=7, B=acetonitrile (ACN): method: 0% B to 80% B in 40 minutes at 13 mL/minute). The pure fractions were pooled and freeze-dried, furnishing the (9H-fluoren-9-yl)methyl (8-oxo-8-((5-sulfamoyl-1,3,4-thiadiazol-2-yl)amino)octyl)carbamate D.

Synthesis of 8-amino-N-(5-sulfamoyl-1,3,4-thiadiazol-2-yl)octanamide E: (9H-fluoren-9-yl)methyl (8-oxo-8-((5-sulfamoyl-1,3,4-thiadiazol-2-yl)amino)octyl)carbamate D (0.080 g) was dissolved in 20% piperidine in DMF (1.0 mL) at room temperature and stirring was continued for 2 hour under argon (Scheme 3). The reaction was monitored by LCMS and purified by preparative HPLC (mobile phase: A=20 mM ammonium acetate pH=7, B=ACN; method: 0% B to 50% B in 30 minutes at 13 mL/minute). The pure fractions were pooled and freeze-dried, furnishing the 8-amino-N-(5-sulfamoyl-1,3,4-thiadiazol-2-yl)octanamide E.

Synthesis of Aza-FITC F: DIPEA (3.0 equiv) at room temperature was added to a stirred solution of 8-amino-N-(5-sulfamoyl-1,3,4-thiadiazol-2-yl)octanamide E (0.0160 g) and FITC (0.019 g, 1.0 equiv., added portion wise 0.80 equiv.+0.20 equiv. over 1 hours) in dimethylsulfoxide (DMSO) (1 mL) and stirred continuously for 3 hours under argon. The reaction was monitored by LCMS and purified by preparative HPLC (mobile phase: A=20 mM ammonium acetate pH=7, B=ACN; method: 0% B to 60% B in 40 min at 13 m/min). The pure fractions were pooled and freeze-dried, furnishing the Aza-FITC F. LCMS (ESI) (m/z): (M+H)⁻ calcd for C₃₁H₃₀N₆O₈S₃+H=711.13 found (m/z): (M+H)⁺710.80.

### Example 19

### Synthesis of CAL-PEGs-FITC Conjugates

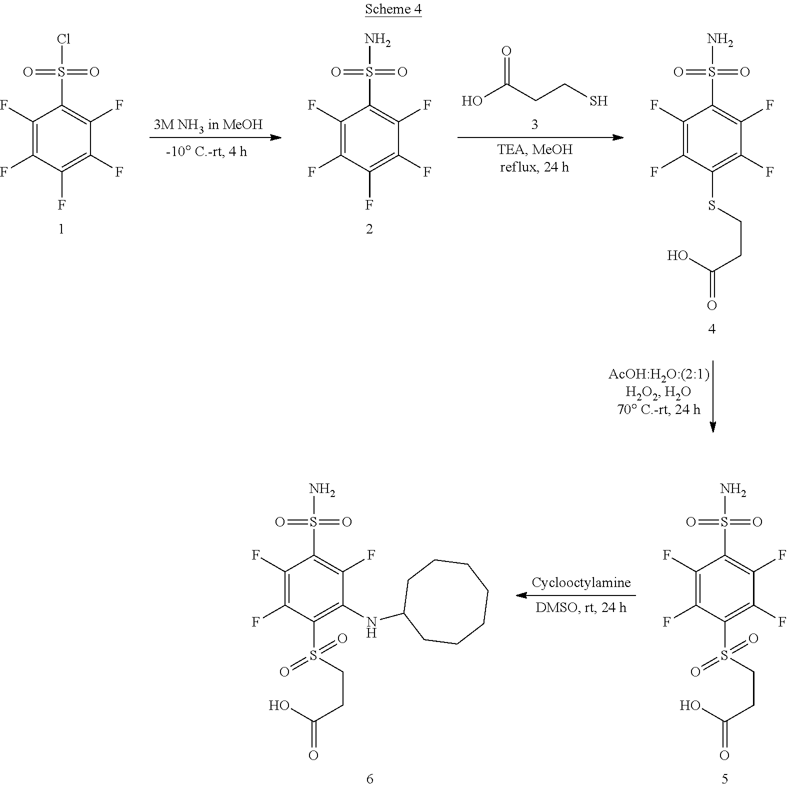 [SMILES: O=S(=O)(Cl)c1c(F)c(F)c(F)c(F)c1F ; NS(=O)(=O)c1c(F)c(F)c(F)c(F)c1F ; O=C(O)CCS ; NS(=O)(=O)c1c(F)c(F)c(SCCC(=O)O)c(F)c1F ; NS(=O)(=O)c1c(F)c(F)c(S(=O)(=O)CCC(=O)O)c(NC2CCCCCCC2)c1F ; NS(=O)(=O)c1c(F)c(F)c(S(=O)(=O)CCC(=O)O)c(F)c1F]

### Synthesis of meta-CAL-P3-FITC conjugate

meta-CAL-PEG₃ NHBoc (8), meta-CA ligand 6 was synthesized by the known procedure described in literature. More specifically, to a stirred solution of meta-CA ligand 6 (0.039 g, 1.0 equiv), t-Boc-N-amido-PEG₃ amine 7 (0.027 g 1.1 equiv) and hexafluorophosphate azabenzotriazole tetramethyl uranium (HATU) (0.036 g, 1.15 equiv) in DMF (1.5 mL). DIPEA (0.0173 mL, 1.2 equiv) was added at room temperature under argon, and the solution was stirred for 3 to 4 hours as mentioned in Scheme 5. The reaction was monitored by LCMS and purified by C-18 column reverse phase (mobile phase: A=20 mM ammonium acetate pH=7, B=ACN; method: 5% B to 95% B in 50 minutes). The pure fractions were pooled and freeze-dried, furnishing the meta-CAL-PEG₃ NHBoc 8.

meta-CAL-PEG₃ NH₂ (9), meta-CAL-PEG3 NHBoc 8 (0.050 g) was dissolved 30% TFA in wet DCM (1.5 mL) at room temperature and stirred for 1 to 2 hours (Scheme 5). The reaction was monitored by LCMS and volatiles were removed by rotavapor. The residue was dissolved in DCM and purified by column chromatography on silica gel (DCM:Methanol:TFA=89:10:1). Further, trace impurity was purified by preparative thin-layer chromatography (TLC) (DCM:Methanol:TFA=89:10:1) to afford meta-CAL-PEG₃NH₂ 9.

meta-CAL-P3-FITC (11), meta-CAL-PEG3NH₂ 8 (0.041 g, 1.0 equiv.) was dissolved in DMF (1.0 mL) under argon atmosphere, after that FITC 10 (0.024 g, 1.0 equiv. added portion wise 0.80 equiv.+0.20 equiv. over 2 hours) and DIPEA (1.0 equiv.) was added at room temperature under dark condition (Scheme 5). The reaction mixture was stirred for an additional hour. The reaction was monitored by LCMS and was purified by C-18 column reverse phase (mobile phase: A=milli Q water (TFA) pH=3, B=ACN; method: 5% B to 95% B in 50 minutes). The pure fraction was collected, froze at −80° C. for three hours, and lyophilized to yield meta-CAL-P3-FITC conjugate 11. The purity of compound meta-CAL-P3-FITC conjugate 11 was analyzed by LCMS.

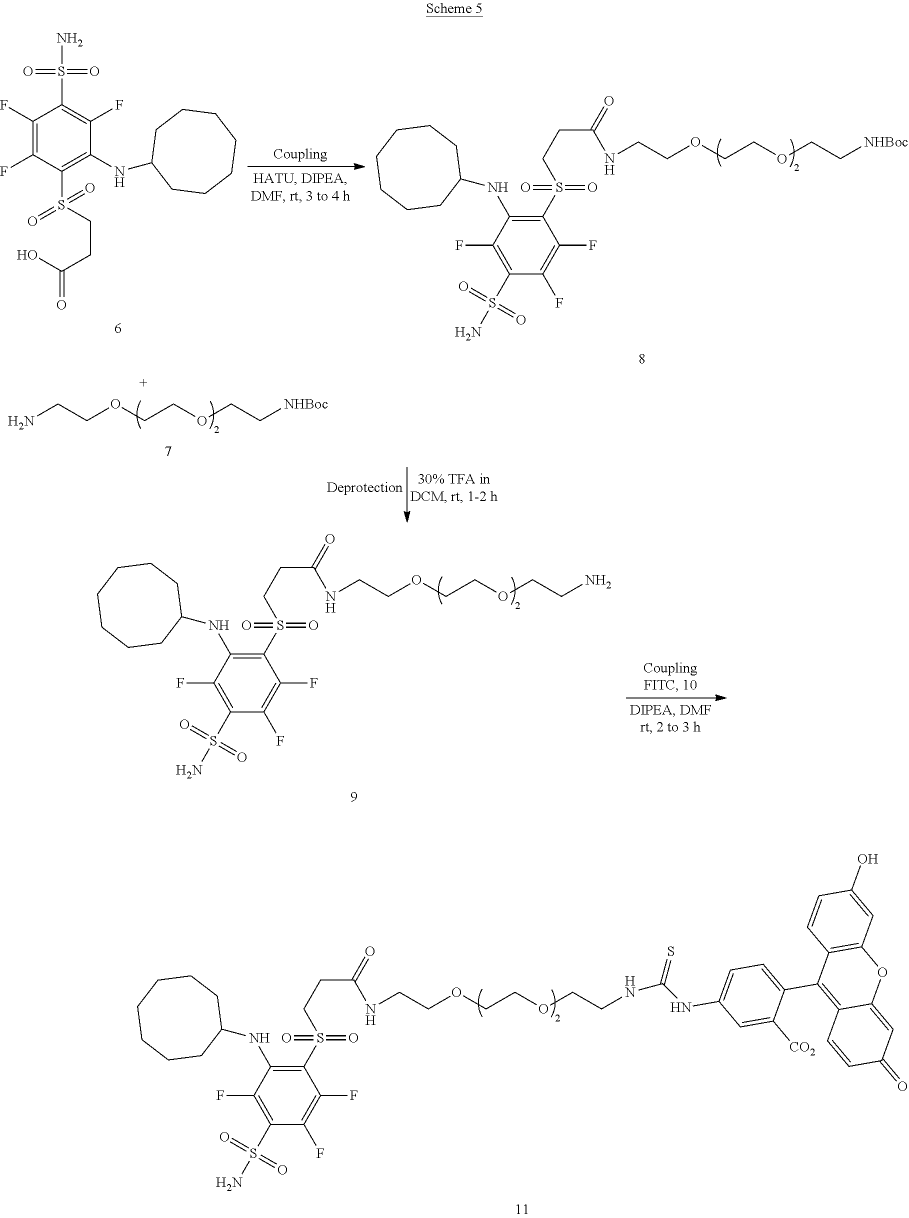

### Synthesis of Meta-CAL-P6-FITC Conjugate

meta-CAL-PEG₆ NHBoc (13). DIPEA (0.0177 mL, 1.2 equiv) was added at room temperature under argon atmosphere to a stirred solution of meta-CA ligand 6 (0.040 g, 1.0 equiv), t-Boc-N-amido-PEG₆ amine 12 (0.039 g 1.1 equiv) and HATU (0.037 g, 1.15 equiv) in DMF (1.5 mL), and the solution was stirred for 3 to 4 hours as mentioned in Scheme 6. The reaction was monitored by LCMS and purified by C-18 column reverse phase (mobile phase: A=20 mM ammonium acetate pH=7, B=ACN; method: 5% B to 95% B in 50 minutes). The pure fractions were pooled and freeze-dried, furnishing the meta-CAL-PEG₆ NHBoc 13.

meta-CAL-PEG₆ NH₂ (14), meta-CAL-PEG₆ NHBoc 13 (0.056 g, 1.0 equiv.) was dissolved 30% TFA in wet DCM (1.5 mL) at room temperature and stirred for 1 to 2 hours (Scheme 6). The reaction was monitored by LCMS and volatiles were removed by rotavapor. The residue was dissolved in DCM and purified by column chromatography on silica gel (DCM:Methanol:TFA=94:5:1). Further, trace impurity was purified by preparative TLC (DCM:Methanol:TFA=94:5:1) to afford meta-CAL-PEG₆ NH₂ 14.

meta-CAL-P6-FITC (15), meta-CAL-PEG₆NH₂ 14 (0.057 g, 1.0 equiv.) was dissolved in DMF (1.0 mL) under argon atmosphere, after that FITC 10 (0.028 g, 1.0 equiv. added portion wise 0.80 equiv.+0.20 equiv. over 2 hours) and DIPEA (1.0 equiv.) was added at room temperature under dark condition (Scheme 6). The reaction mixture was stirred for an additional hour. The reaction was monitored by LCMS and was purified by C-18 column reverse phase (mobile phase: A=milli Q water (TFA) pH=3, B=ACN; method: 5% B to 95% B in 50 minutes). The pure fraction was collected, frozen at −80° C. for three hours, and lyophilized to yield meta-CAL-P6-FITC conjugate 15. The purity of compound meta-CAL-P6-FITC conjugate 15 was analyzed by LCMS.

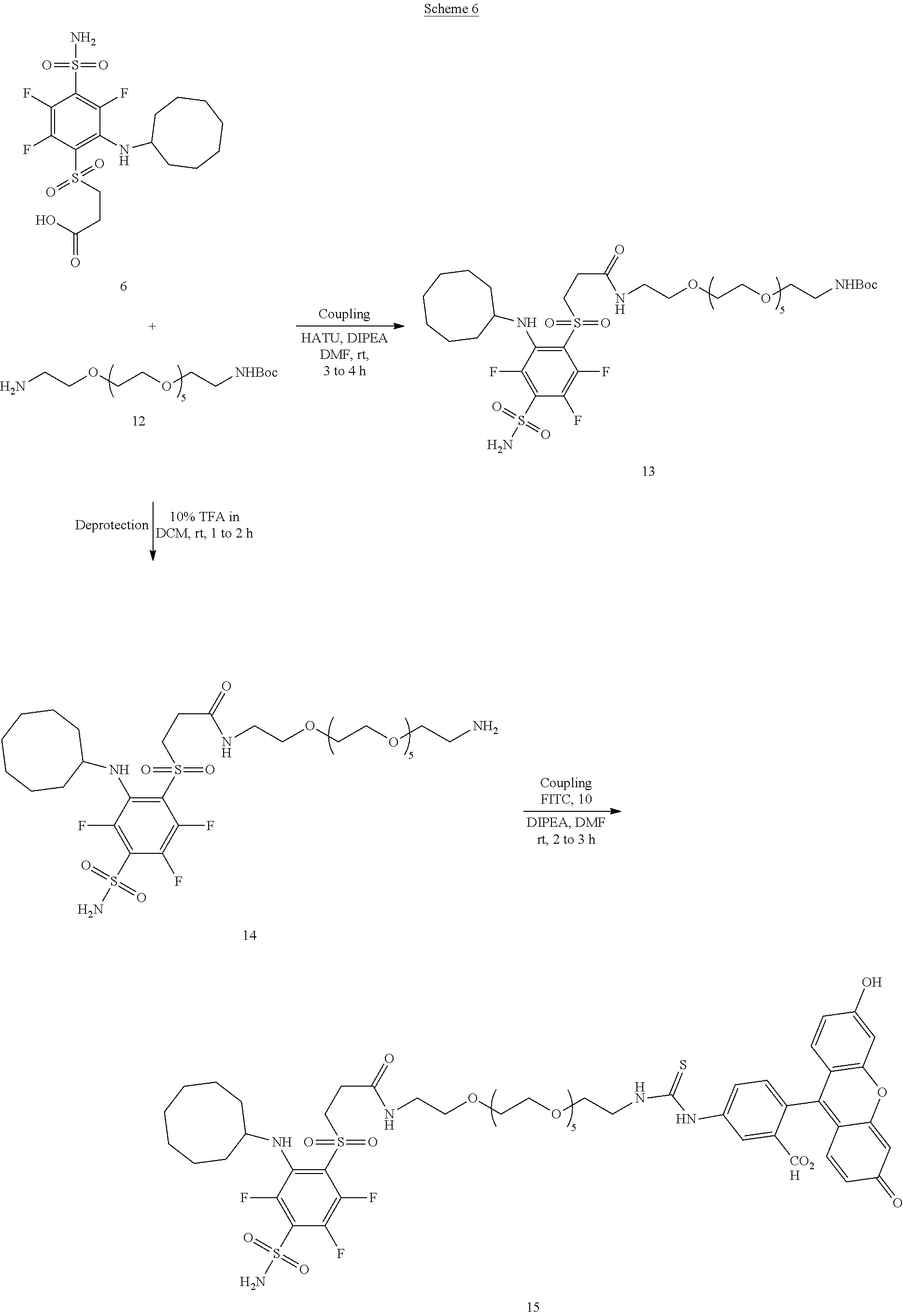

### Synthesis of Meta-CAL-P9-FITC Conjugate

meta-CAL-PEG₉ NHBoc (17). To a stirred solution of meta-CA ligand 6 (0.031 g, 1.0 equiv), t-Boc-N-amido-PEG₉ amine 16 (0.040 g 1.1 equiv) and HATU (0.028 g, 1.15 equiv) in DMF (1.0 mL) was added DIPEA (0.014 mL, 1.2 equiv) at room temperature under argon, and the solution was stirred for 3 to 4 hours as mentioned in Scheme 7. The reaction was monitored by LCMS and purified by C-18 column reverse phase (mobile phase: A=20 mM ammonium acetate pH=7, B=ACN; method: 5% B to 95% B in 50 minutes). The pure fractions were pooled and freeze-dried, furnishing the meta-CAL-PEG₉ NHBoc 17.

meta-CAL-PEG₉ NH₂ (18), meta-CAL-PEG₉ NHBoc 17 (0.067 g. 1.0 equiv.) was dissolved 30% TFA in wet DCM (1.5 mL) at room temperature and stirred for 1 to 2 hours (Scheme 7). The reaction was monitored by LCMS and volatiles were removed by rotavapor. The residue was dissolved in DCM and purified by column chromatography on silica gel (DCM:Methanol:TFA=94:5:1). Further, trace impurity was purified by preparative TLC (DCM:Methanol:TFA=94:5:1) to afford meta-CAL-PEG₉ NH₂ 18.

meta-CAL-P9-FITC (19), meta-CAL-PEG₉NH₂ 18 (0.041 g, 1.0 equiv.) was dissolved in DMF (1.0 mL) under argon atmosphere, after that FITC 10 (0.017 g, 1.0 equiv. added portion wise 0.80 equiv.+0.20 equiv. over 2 hours) and DIPEA (1.0 equiv.) was added at room temperature under dark condition (Scheme 7). The reaction mixture was stirred for an additional hour. The reaction was monitored by LCMS and was purified by C-18 column reverse phase (mobile phase: A=milli Q water (TFA) pH=3, B=ACN; method: 5% B to 95% B in 50 minutes). The pure fraction was collected, froze at −80° C. for three hours, and lyophilized to yield meta-CAL-P9-FITC conjugate 19. The purity of compound meta-CAL-P9-FITC 19 was analyzed by LCMS

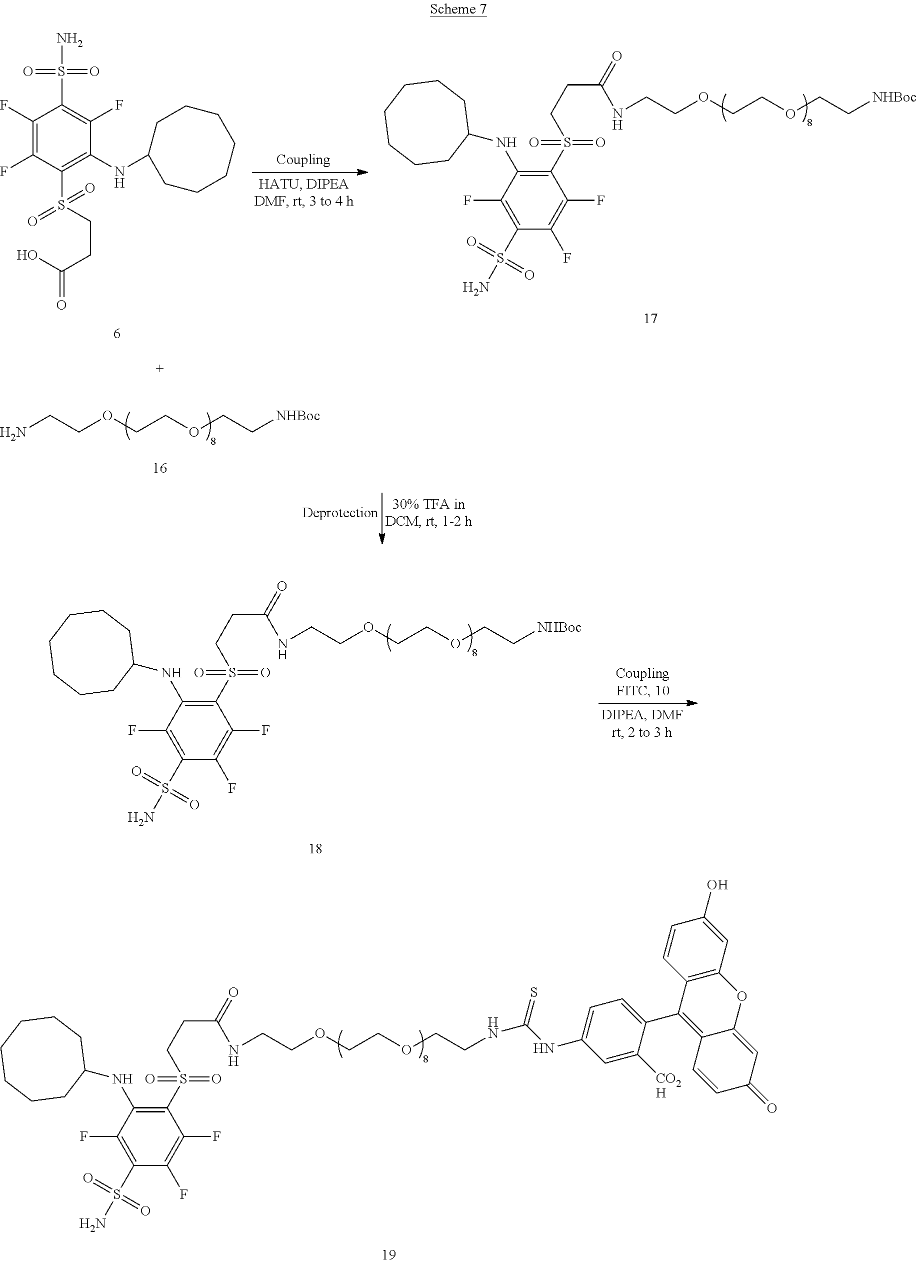

### Synthesis of Ortho-CAL-P6-FITC Conjugate

ortho-CAL-PEG₆ NHBoc (21). To a stirred solution of ortho-CA ligand 20 (0.033 g, 1.0 equiv), t-Boc-N-amido-PEG₆ amine 12 (0.032 g 1.1 equiv) and HATU (0.033 g, 1.15 equiv) in DMF (1.5 mL) was added DIPEA (0.015 mL, 1.2 equiv) at room temperature under argon, and the solution was stirred for 3 to 4 hours as mentioned in Scheme 8. The reaction was monitored by LCMS and purified by C-18 column reverse phase (mobile phase: A=20 mM ammonium acetate pH=7, B=ACN; method: 5% B to 95% B in 50 minutes). The pure fractions were pooled and freeze-dried, furnishing the ortho-CAL-PEG₆ NHBoc 21.

ortho-CAL-PEG₆ NH₂ (22), ortho-CAL-PEG₆ NHBoc 21 (0.110 g, 1.0 equiv.) was dissolved 25% TFA in wet DCM (1.5 mL) at room temperature and stirred for 2 hours (Scheme 8). The reaction was monitored by LCMS, volatiles were removed by rotavapor. The residue was dissolved in DMF purified by C-18 column reverse phase (mobile phase: A=20 mM ammonium acetate pH=7, B=ACN; method: 5% B to 95% B in 60 minutes). The pure fractions were pooled and freeze-dried, furnishing the ortho-CAL-PEG₆NH₂ 22.

ortho-CAL-P6-FITC (23), ortho-CAL-PEG₆NH₂ 22 (0.060 g. 1.0 equiv.) was dissolved in DMF (1.0 mL) under argon atmosphere, after that FITC 10 (0.028 g, 0.9 equiv. added portion wise 0.70 equiv.+0.20 equiv. over an hour) and DIPEA (1.25 equiv.) was added at room temperature under dark condition (Scheme 8). The reaction mixture was stirred for an additional hour. The reaction was monitored by LCMS and was purified by C-18 column reverse phase (mobile phase: A=milli Q water (TFA) pH=3, B=ACN; method: 5% B to 95% B in 50 minutes). The pure fraction was collected, froze at −80° C. for three hours, and lyophilized to yield ortho-CAL-P6-FITC conjugate 23. The purity of compound ortho-CAL-P6-FITC 23 was analyzed by LCMS.

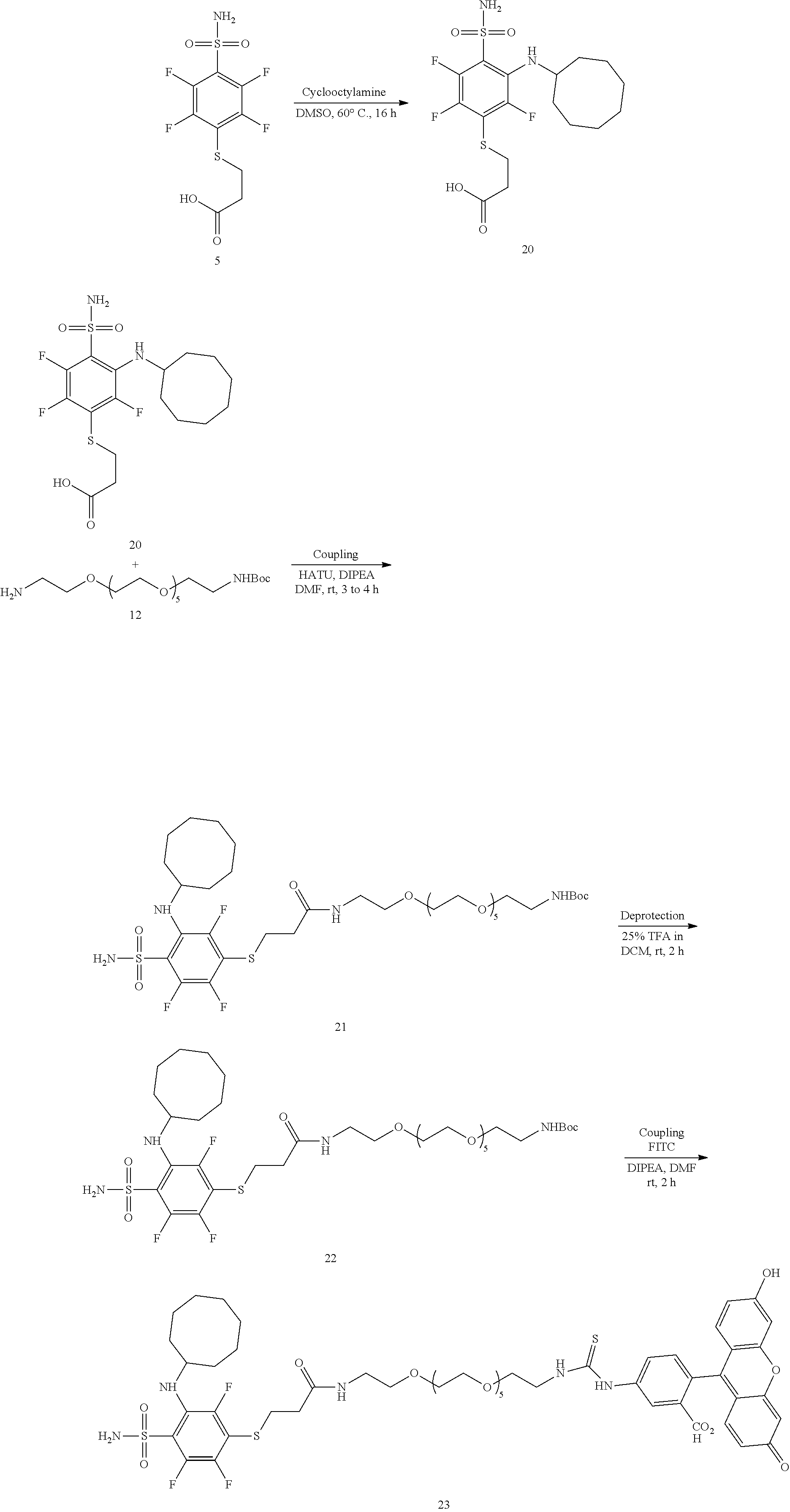

### Synthesis of Ortho-CAL-P9-FITC Conjugate

ortho-CAL-PEG₉ NHBoc (24), ortho-CA ligand 20 was synthesized by the known procedure described in literature. In particular, to a stirred solution of ortho-CA ligand 20 (0.022 g, 1.0 equiv), t-Boc-N-amido-PEG₉ amine 16 (0.028 g 1.0 equiv) and HATU (0.022 g, 1.15 equiv) in DMF (1.0 mL), DIPEA (0.0104 mL, 1.2 equiv) was added at room temperature under argon atmosphere, and the solution was stirred for 3 to 4 hours as mentioned in Scheme 9. The reaction was monitored by LCMS and purified by C-18 column reverse phase (mobile phase: A=20 mM ammonium acetate pH=7, B=ACN; method: 5% B to 95% B in 50 minutes). The pure fractions were pooled and freeze-dried, furnishing the ortho-CAL-PEG₉ NHBoc 24.

ortho-CAL-PEG₉ NH₂ (25), ortho-CAL-PEG₉ NHBoc 24 (0.098 g, 1.0 equiv.) was dissolved 25% TFA in wet DCM (1.5 mL) at room temperature and stirred for 2 hours (Scheme 9). The reaction was monitored by LCMS and volatiles were removed by rotavapor. The residue was dissolved in DMF and purified by C-18 column reverse phase (mobile phase: A=20 mM ammonium acetate pH=7, B=ACN; method: 5% B to 95% B in 60 minutes). The pure fractions were pooled and freeze-dried, furnishing the ortho-CAL-PEG₉NH₂ 25.

ortho-CAL-P9-FITC (26), ortho-CAL-PEG₉Nhour (0.071 g, 1.0 equiv.) was dissolved in DMF (1.0 mL) under argon atmosphere, after that FITC 10 (0.028 g, 0.9 equiv. added portion wise 0.70 equiv.+0.20 equiv. over an hours) and DIPEA (1.25 equiv.) was added at room temperature under dark condition (Scheme 9). The reaction mixture was stirred for an additional hour.

The reaction was monitored by LCMS and was purified by C-18 column reverse phase (mobile phase: A=milli Q water (TFA) pH=3, B=ACN; method: 5% B to 95% B in 50 minutes). The pure fraction was collected, frozen at −80° C. for 3 hours, and lyophilized to yield ortho-CAL-P9-FITC conjugate 26. The purity of compound ortho-CAL-P9-FITC 26 was analyzed by LCMS.

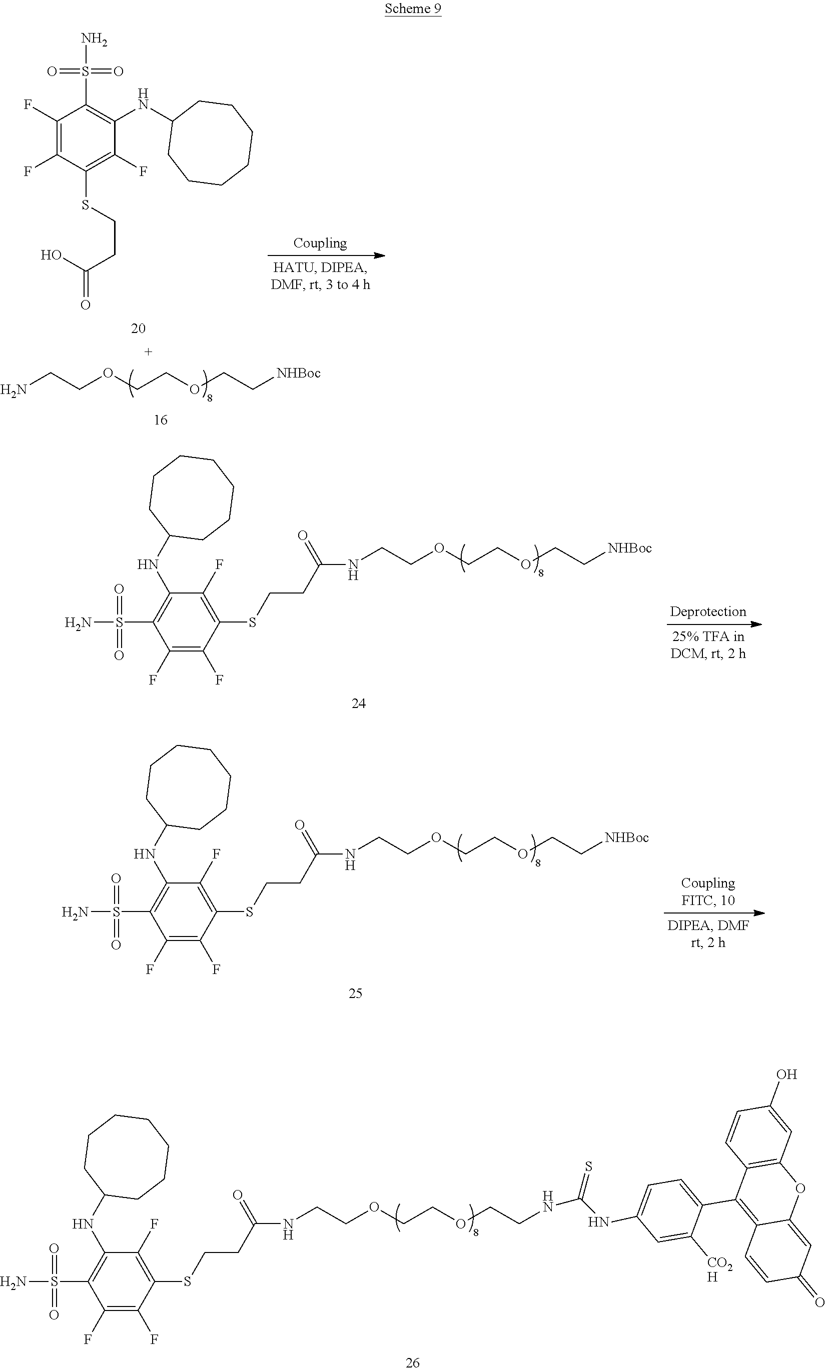

### ENUMERATED EMBODIMENTS OF THE CLAIMED INVENTION

Clause 1. A bispecific adapter or a pharmaceutically acceptable salt or hydrate thereof comprising a fluorescein, fluorescein isothiocyanate (FITC), or N-hydroxysuccinimide (NHS)-fluorescein conjugated to a radical of a carbonic anhydrase IX (CAIX) ligand via a linker, wherein the linker comprises, consists essentially of, or consists of polyethylene glycol (PEG).
Clause 2. The bispecific adapter of clause 1, wherein the CAIX ligand is or comprises 3-((3-(cyclooctylamino)-2,5,6-trifluoro-4-sulfamoylphenyl)thio)propanoic acid (ortho-CAL), 3-((2-(cyclooctylamino)-3,5,6-trifluoro-4-sulfamoylphenyl) sulfonyl) propanoic acid (meta-CAL), acetazolamide (Aza), or a derivative or analog of any of the foregoing.
Clause 3. The bispecific adapter of clause 1, wherein the linker comprises, consists essentially of, or consists of PEG₁ to PEG₉.
Clause 4. The bispecific adapter of clause 1, wherein the CAIX ligand is or comprises ortho-CAL or a derivative or analog thereof and the linker comprises, consists essentially of, or consists of PEG₁ to PEG₉.
Clause 5. The bispecific adapter of clauses 1-3, wherein the linker comprises, consists essentially of, or consists of PEG₃ to PEG₉.
Clause 6. The bispecific adapter of clauses 1-3, wherein the CAIX ligand is or comprises meta-CAL or a derivative or analog thereof and the linker comprises, consists essentially of, or consists of PEG₃ to PEG₉.
Clause 7. The bispecific adapter of clause 3, wherein the CAIX ligand is or comprises Aza or a derivative or analog thereof.
Clause 8. The bispecific adapter of any one of clauses 4, 6, and 7, wherein the linker comprises, consists essentially of, or consists of PEG₆.
Clause 9. The bispecific adapter of clause 6 or clause 7, wherein the linker comprises, consists essentially of, or consists of PEG₉.
Clause 10. The bispecific adapter of clause 7, wherein the linker comprises, consists essentially of, or consists of PEG₆.
Clause 11. The bispecific adapter of any of clauses 1-10, wherein the linker comprises, consists essentially of, or consists of an alkyl.
Clause 12. The bispecific adapter of any one of clauses 1-10, wherein the linker comprises, consists essentially of, or consists of (CH₂)₄.
Clause 13. The bispecific adapter of clause 1, comprising a structure of one of the following formulae:

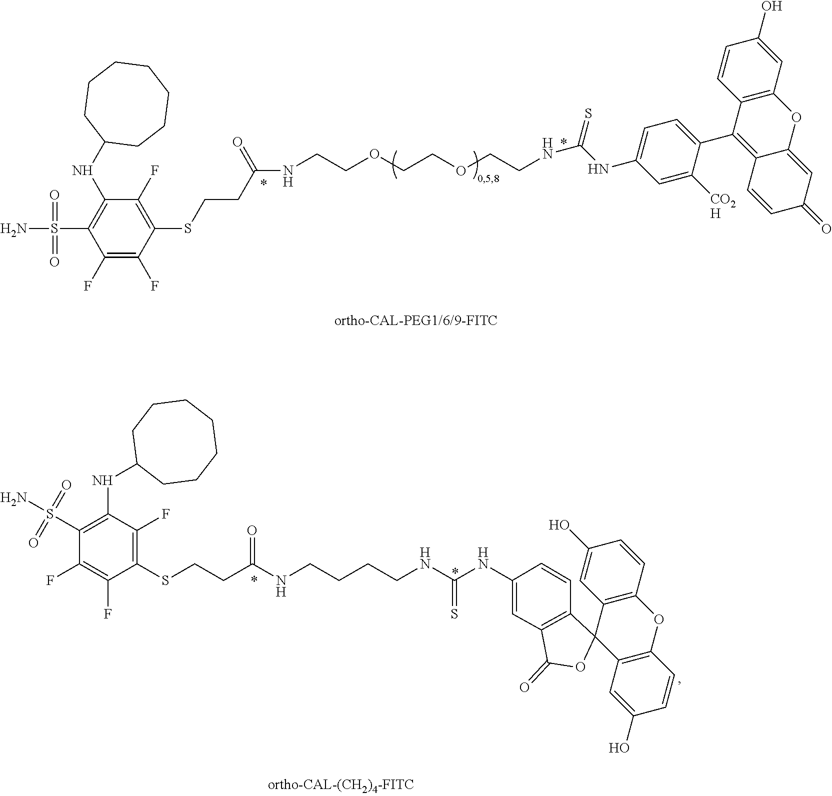

or a pharmaceutically acceptable salt or hydrate of either of the foregoing.
Clause 14. The bispecific adapter of clause 1 comprising a structure of the following formulae:

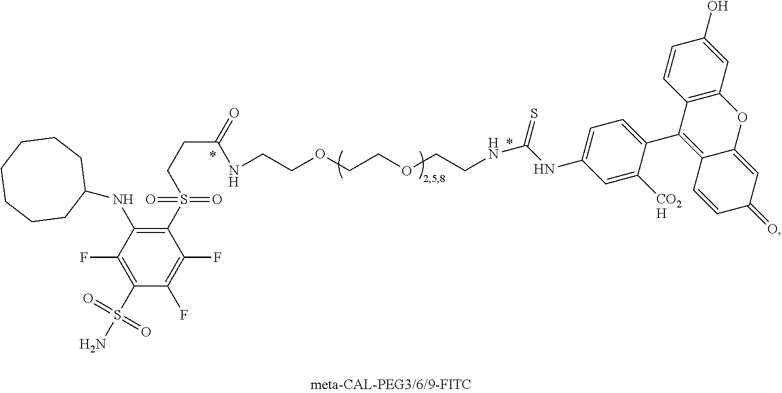

or a pharmaceutically acceptable salt or hydrate thereof.
Clause 15. The bispecific adapter of clause 1, comprising a structure of the following formulae:

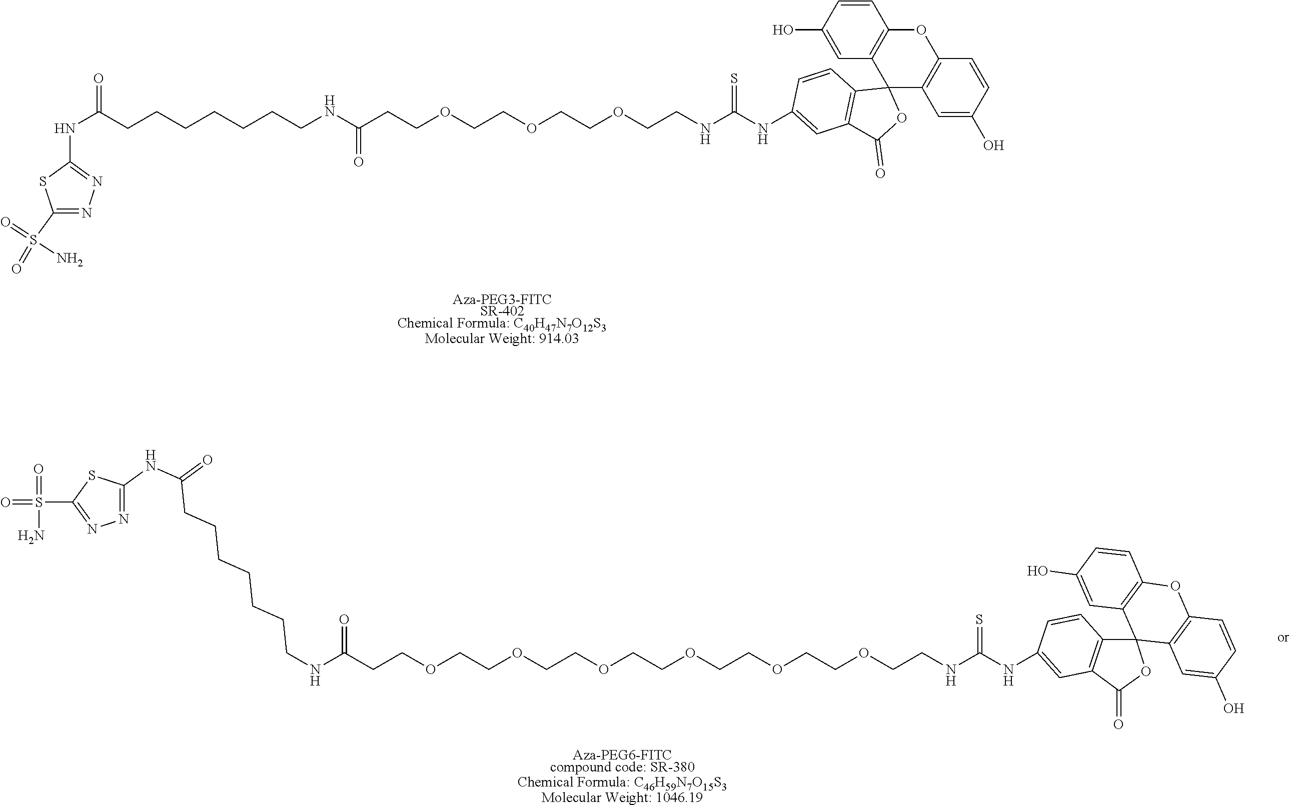

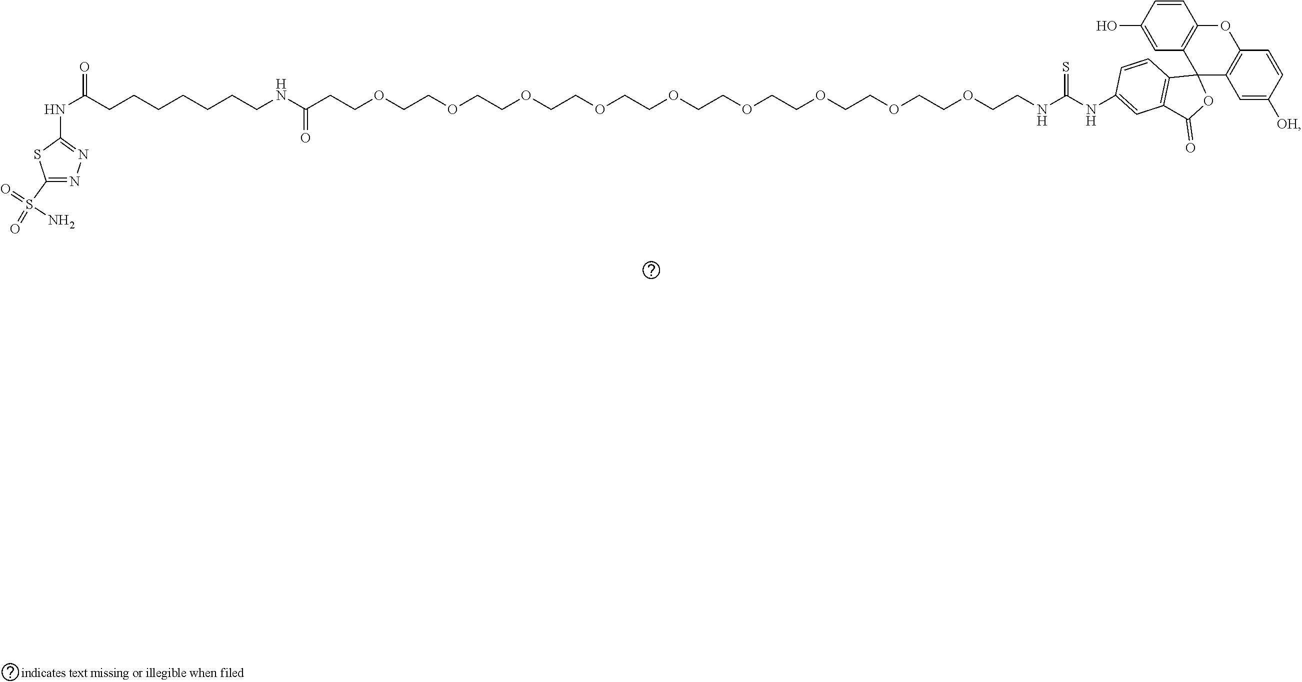 [SMILES: NS(=O)(=O)c1nnc(NC(=O)CCCCCCCNC(=O)CCOCCOCCOCCOCCOCCOCCOCCOCCOCCNC(=S)Nc2ccc3c(c2)C(=O)OC32c3cc(O)ccc3Oc3ccc(O)cc32)s1]

or a pharmaceutically acceptable salt or hydrate of any of the foregoing.
Clause 16. The bispecific adapter of any one of clauses 1-15 for use with an anti-fluorescein chimeric antigen receptor (CAR)-T cell in the treatment of cancer.
Clause 17. The bispecific adapter of any one of clauses 1-15 for use with an anti-fluorescein CAR-T cell in the treatment of a CAIX-expressing cancer.
Clause 18. A pharmaceutical composition for the treatment of a CAIX-expressing cancer comprising the bispecific adapter of any one of clauses 1-16 and a pharmaceutically acceptable carrier or excipient.
Clause 19. A kit comprising:
(i) at least one dosage unit of a bispecific adapter of any one of clauses 1-17 or a pharmaceutical composition of clause 18; and
(ii) at least one dosage unit of anti-fluorescein chimeric antigen receptor (CAR)-T cells or a pharmaceutical composition comprising anti-fluorescein CAR-T cells and a pharmaceutically acceptable carrier or excipient;
wherein (i) and (ii) are optionally in separate containers.
Clause 20. The kit of clause 19, wherein the CAIX ligand of the bispecific adapter is or comprises ortho-CAL or a derivative or analog thereof
Clause 21. The kit of clause 19, wherein the CAIX ligand of the bispecific adapter is or comprises meta-CAL or a derivative or analog thereof.
Clause 22. The kit of clause 19, wherein the CAIX of the bispecific adapter is or comprises Aza or a derivative or analog thereof
Clause 23. A method of treating cancer in a subject comprising administering to the subject cancer-treatment effective amounts of:
(i) anti-fluorescein CAR-T cells or a pharmaceutical composition comprising anti-fluorescein CAR-T cells and a pharmaceutically acceptable carrier or excipient; and
(ii) a bispecific adapter of any one of clauses 1-17 or a pharmaceutical composition of claim 18;
whereupon the subject is treated for cancer.
Clause 24. The method of clause 23, wherein the CAR comprises: a recognition region comprising a single chain fragment variable (scFv) region of an anti-fluorescein antibody; (a co-stimulation domain, which is CD28, CD137 (4-1BB), CD134 (OX40), or CD278 (ICOS); and/or an activation signaling domain, which is a T cell CD3 chain or an Fc receptor γ.
Clause 25. The method of clause 23, wherein the fluorescein of the bispecific adapter binds the anti-fluorescein CAR-T cell with affinity upon exposure thereto, and the CAIX ligand of the bispecific adapter links the bound anti-fluorescein CAR-T cell to a CAIX-expressing cancer cell upon the bispecific adapter binding a receptor on such CAIX-expressing cancer cell with affinity.
Clause 26. The method of clause 23, wherein (i) and (ii) are administered simultaneously or sequentially, in either order, by the same or different routes.
Clause 27. The method of any one of clauses 23-26, wherein (i) and (ii) are each administered intravenously.
Clause 28. The method of any one of clauses 23-27, further comprising imaging the cancer in the subject.
Clause 29. The method of clause 28, wherein imaging the cancer comprises imaging by optical imaging, positron emission tomography (PET), or single photon emission computed tomography (SPECT).
Clause 30. The method of any one of clauses 23-29, wherein the cancer is a CAIX-expressing cancer.
Clause 31. The method of any one of clauses 23-30, wherein the cancer is ovarian cancer, endometrial cancer, breast cancer, lung cancer, bladder cancer, or clear cell renal cell carcinoma such as, optionally, stage 3-4 clear cell renal cell carcinoma.
Clause 32. A method for enhancing chimeric antigen receptor (CAR)-T cell activation comprising:
(i) providing a bispecific adapter of any one of clauses 1-17 or a pharmaceutical composition of clause 18; and
(ii) exposing anti-fluorescein CAR-T cells or a pharmaceutical composition comprising anti-fluorescein CAR-T cells and a pharmaceutically acceptable carrier or excipient to the bispecific adaptor or pharmaceutical composition;
wherein the CAR-T cell experiences enhanced activation against cancer cells following exposure as compared to a CAR-T cell not exposed to the bispecific adapter.
Clause 33. The method of clause 32, wherein the anti-fluorescein CAR-T cells are in systemic circulation in a subject when exposed to the bispecific adaptor.
Clause 34. The method of clause 32, wherein the cancer cells are CAIX-expressing cancer cells.

1. A bispecific adapter or a pharmaceutically acceptable salt or hydrate thereof comprising a fluorescein, fluorescein isothiocyanate (FITC), or N-hydroxysuccinimide (NHS)-fluorescein conjugated to a radical of a carbonic anhydrase IX (CAIX) ligand via a linker, wherein the linker comprises, consists essentially of, or consists of polyethylene glycol (PEG).

2. The bispecific adapter of claim 1, wherein the CAIX ligand is or comprises 3-((3-(cyclooctylamino)-2,5,6-trifluoro-4-sulfamoylphenyl)thio)propanoic acid (ortho-CAL), 3-((2-(cyclooctylamino)-3,5,6-trifluoro-4-sulfamoylphenyl) sulfonyl) propanoic acid (meta-CAL), acetazolamide (Aza), or a derivative or analog of any of the foregoing.

3. The bispecific adapter of claim 1, wherein the linker comprises, consists essentially of, or consists of PEG₁ to PEG₉.

4. The bispecific adapter of claim 1, wherein the CAIX ligand is or comprises ortho-CAL or a derivative or analog thereof and the linker comprises, consists essentially of, or consists of PEG₁ to PEG₉.

5. The bispecific adapter of claim 1, wherein the linker comprises, consists essentially of, or consists of PEG₃ to PEG₉.

6. The bispecific adapter of claim 1, wherein the CAIX ligand is or comprises meta-CAL or a derivative or analog thereof and the linker comprises, consists essentially of, or consists of PEG₃ to PEG₉.

7. The bispecific adapter of claim 3, wherein the CAIX ligand is or comprises Aza or a derivative or analog thereof.

8. The bispecific adapter of any one of claims 4, 6, and 7, wherein the linker comprises, consists essentially of, or consists of PEG₆.

9. The bispecific adapter of claim 6 or claim 7, wherein the linker comprises, consists essentially of, or consists of PEG₉.

10. The bispecific adapter of claim 7, wherein the linker comprises, consists essentially of, or consists of PEG₆.

11. The bispecific adapter of claim 1, wherein the linker comprises, consists essentially of, or consists of an alkyl.

12. The bispecific adapter of claim 1, wherein the linker comprises, consists essentially of, or consists of (CH₂)₄.

13. The bispecific adapter of claim 1, comprising a structure of one of the following formulae: 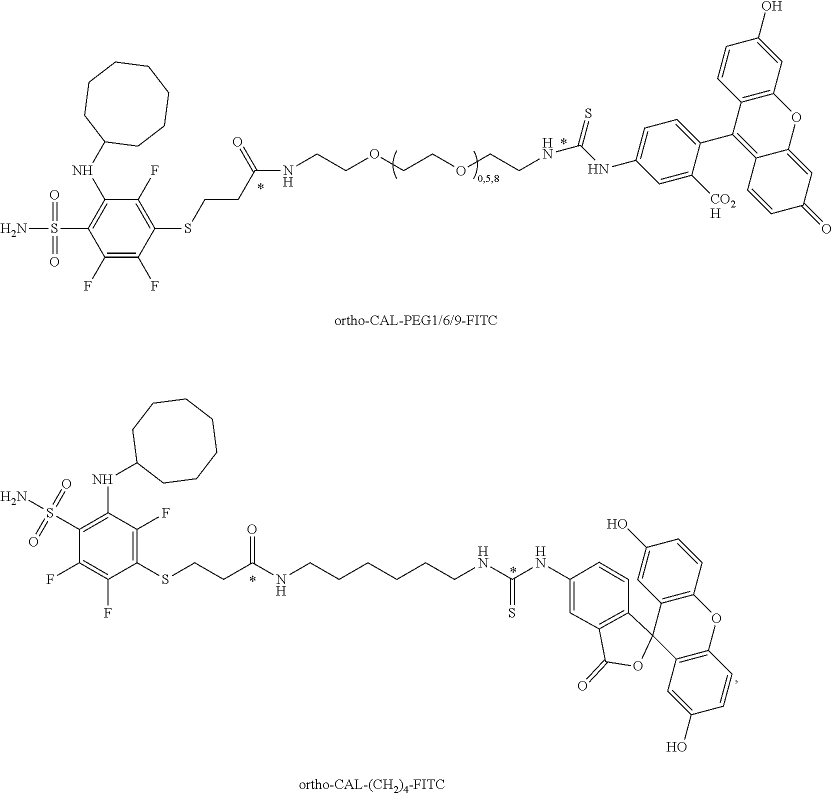
or a pharmaceutically acceptable salt or hydrate of either of the foregoing.

14. The bispecific adapter of claim 1 comprising a structure of the following formulae: 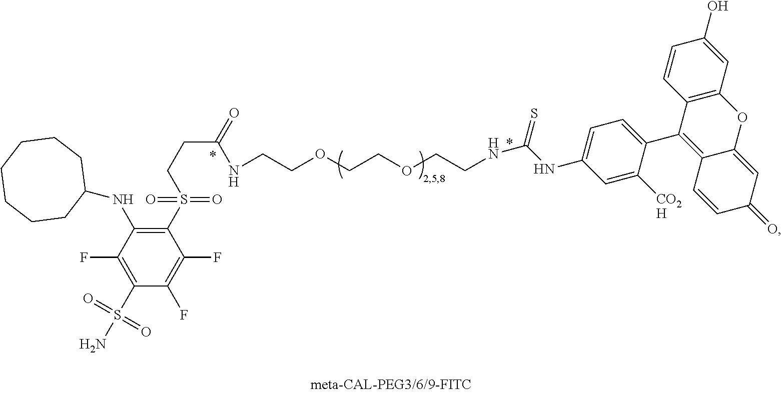
or a pharmaceutically acceptable salt or hydrate thereof.

15. The bispecific adapter of claim 1, comprising a structure of the following formulae: 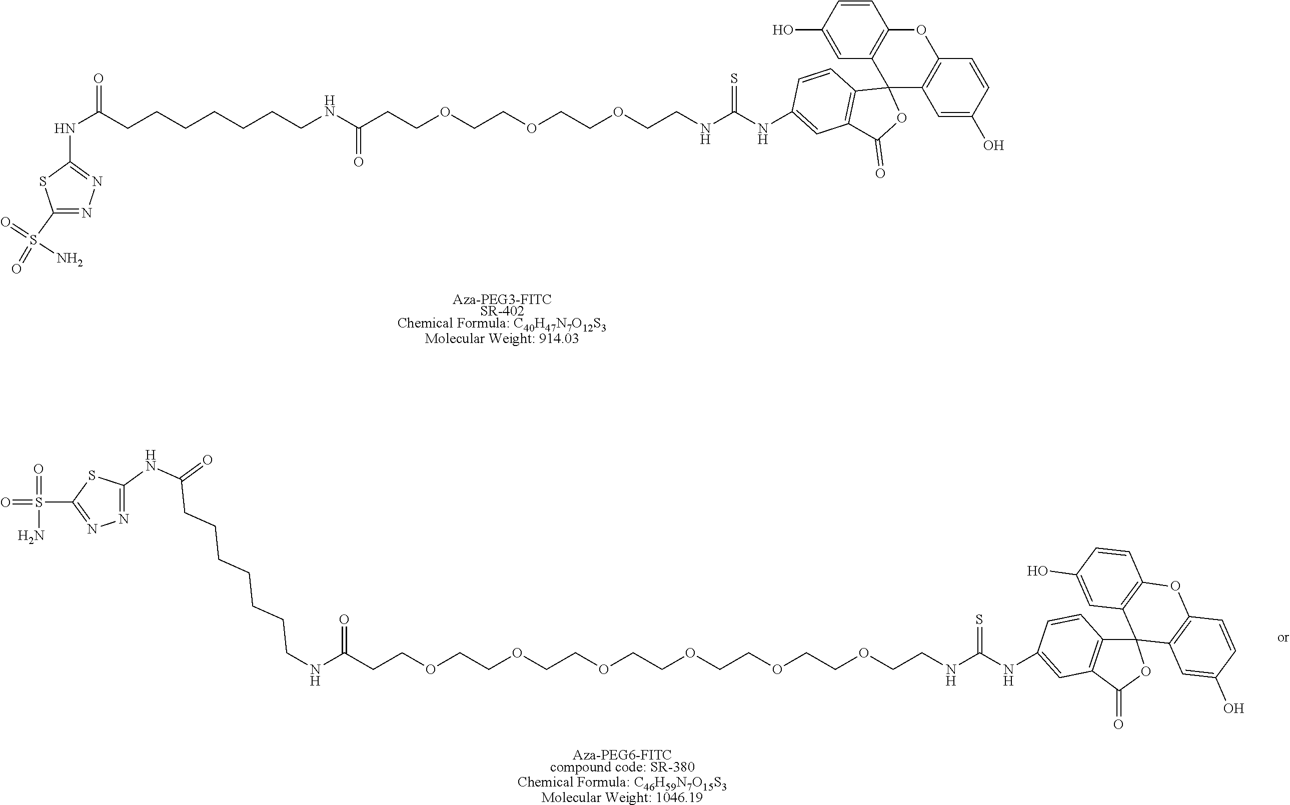 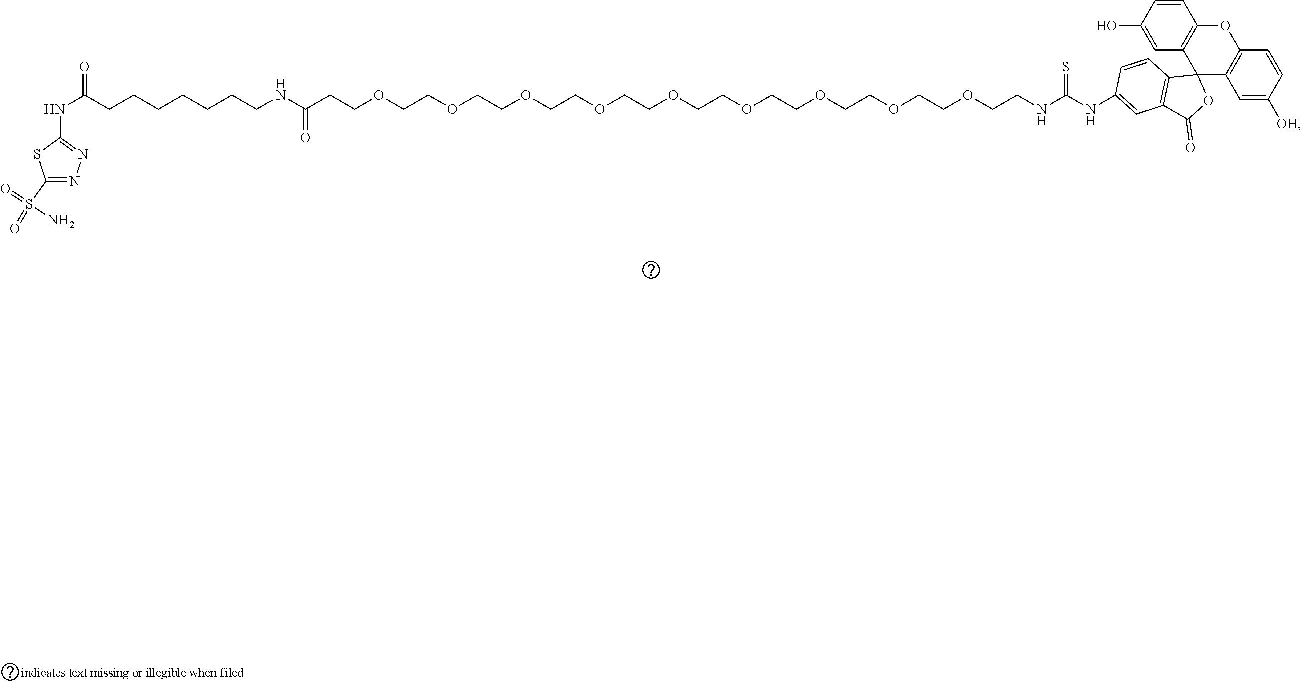 [SMILES: NS(=O)(=O)c1nnc(NC(=O)CCCCCCCNC(=O)CCOCCOCCOCCOCCOCCOCCOCCOCCOCCNC(=S)Nc2ccc3c(c2)C(=O)OC32c3cc(O)ccc3Oc3ccc(O)cc32)s1]
or a pharmaceutically acceptable salt or hydrate of any of the foregoing.

16. The bispecific adapter of any one of claims 1-7 and 10-15 for use with an anti-fluorescein chimeric antigen receptor (CAR)-T cell in the treatment of cancer.

17. The bispecific adapter of any one of claims 1-7 and 10-15 for use with an anti-fluorescein CAR-T cell in the treatment of a CAIX-expressing cancer.

18. A pharmaceutical composition for the treatment of a CAIX-expressing cancer comprising the bispecific adapter of any one of claims 1-17 and a pharmaceutically acceptable carrier or excipient.

19. A kit comprising:
(i) at least one dosage unit of a bispecific adapter of any one of claims 1-17 or a pharmaceutical composition of claim 18; and
(ii) at least one dosage unit of anti-fluorescein chimeric antigen receptor (CAR)-T cells or a pharmaceutical composition comprising anti-fluorescein CAR-T cells and a pharmaceutically acceptable carrier or excipient;
wherein (i) and (ii) are optionally in separate containers.

20. The kit of claim 19, wherein the CAIX ligand of the bispecific adapter is or comprises 3-((3-(cyclooctylamino)-2,5,6-trifluoro-4-sulfamoylphenyl)thio)propanoic acid (ortho-CAL) or a derivative or analog thereof.

21. The kit of claim 19, wherein the CAIX ligand of the bispecific adapter is or comprises 3-((2-(cyclooctylamino)-3,5,6-trifluoro-4-sulfamoylphenyl) sulfonyl) propanoic acid (meta-CAL) or a derivative or analog thereof.

22. The kit of claim 19, wherein the CAIX of the bispecific adapter is or comprises acetazolamide (Aza) or a derivative or analog thereof.

23. A method of treating cancer in a subject comprising administering to the subject cancer-treatment effective amounts of:
(i) anti-fluorescein chimeric antigen receptor (CAR)-T cells or a pharmaceutical composition comprising anti-fluorescein CAR-T cells and a pharmaceutically acceptable carrier or excipient; and
(ii) a bispecific adapter of any one of claims 1-17 or a pharmaceutical composition of claim 18;
whereupon the subject is treated for cancer.

24. The method of claim 23, wherein the CAR comprises:
a recognition region comprising a single chain fragment variable (scFv) region of an anti-fluorescein antibody;
a co-stimulation domain, which is CD28, CD137 (4-1BB), CD134 (OX40), or CD278 (ICOS); and/or
an activation signaling domain, which is a T cell CD3ζ chain or an Fc receptor 7.

25. The method of claim 23, wherein the fluorescein of the bispecific adapter binds the anti-fluorescein CAR-T cell with affinity upon exposure thereto, and the CAIX ligand of the bispecific adapter links the bound anti-fluorescein CAR-T cell to a CAIX-expressing cancer cell upon the bispecific adapter binding a receptor on such CAIX-expressing cancer cell with affinity.

26. The method of claim 23, wherein (i) and (ii) are administered simultaneously or sequentially, in either order, by the same or different routes.

27. The method of any one of claims 23-26, wherein (i) and (ii) are each administered intravenously.

28. The method of any one of claims 23-27, further comprising imaging the cancer in the subject.

29. The method of claim 28, wherein imaging the cancer comprises imaging by optical imaging, positron emission tomography (PET), or single photon emission computed tomography (SPECT).

30. The method of any one of claims 23-29, wherein the cancer is a CAIX-expressing cancer.

31. The method of any one of claims 23-30, wherein the cancer is ovarian cancer, endometrial cancer, breast cancer, lung cancer, bladder cancer, or clear cell renal cell carcinoma such as, optionally, stage 3-4 clear cell renal cell carcinoma.

32. A method for enhancing chimeric antigen receptor (CAR)-T cell activation comprising:
providing a bispecific adapter of any one of claims 1-17 or a pharmaceutical composition of claim 18; and
exposing anti-fluorescein CAR-T cells or a pharmaceutical composition comprising anti-fluorescein CAR-T cells and a pharmaceutically acceptable carrier or excipient to the bispecific adaptor or pharmaceutical composition;
wherein the CAR-T cell experiences enhanced activation against cancer cells following exposure as compared to a CAR-T cell not exposed to the bispecific adapter.

33. The method of claim 32, wherein the anti-fluorescein CAR-T cells are in systemic circulation in a subject when exposed to the bispecific adaptor.

34. The method of claim 32, wherein the cancer cells are CAIX-expressing cancer cells.
